(19) United States
(12) Patent Application Publication
(10) Pub. No.: US 2025/0368640 A1
(43) Pub. Date: Dec. 4, 2025

(54) MULTIPLE KINASE DEGRADERS, COMPOSITIONS COMPRISING THE DEGRADER, AND METHODS OF USING THE SAME

(71) Applicant: BIOFRONT LTD, Grand Cayman (KY)

(72) Inventors: Tianwei MA, Beijing (CN); Lichao FANG, Beijing (CN); Miao LIU, Beijing (CN); Ling SONG, Beijing (CN)

(21) Appl. No.: 18/876,234

(22) PCT Filed: Jun. 5, 2023
(86) PCT No.: PCT/CN2023/098363

§ 371 Date: Dec. 18, 2024

(30) Foreign Application Priority Data
Jun. 20, 2022 (WO) PCT/CN2022/099751

(51) Int. Cl. C07D 471/04; A61K 31/513; A61K 45/06

(52) U.S. Cl. CPC C07D 471/04; A61K 31/513; A61K 45/06

(57) ABSTRACT

Provided are compounds of Formula (I), a tautomer thereof, a deuterated derivative of the compound or the tautomer, and a pharmaceutically acceptable salt of the foregoing, compositions comprising the compounds of Formula (I), a tautomer thereof, a deuterated derivative of the compound or the tautomer, and/or a pharmaceutically acceptable salt of the foregoing, and methods of using the same, in treating, for example, the diseases, disorders, or conditions mediated by the degradation of protein kinases, such as Hematopoietic progenitor kinase 1 (HPK1, MAP4K1), Mitogen-activated protein kinases 1/2 (MEK 1/2), Human Fms-like tyrosine kinase 3 receptor (FLT3), and Aurora kinases.

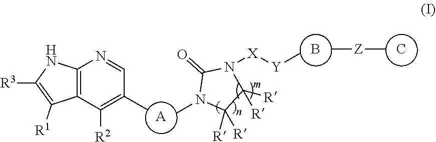

### FIELD OF THE INVENTION

This disclosure provides compounds of Formula I, a tautomer thereof, a deuterated derivative of the compound or the tautomer, and a pharmaceutically acceptable salt of the foregoing, compositions comprising the compounds of Formula I, a tautomer thereof, a deuterated derivative of the compound or the tautomer, and/or a pharmaceutically acceptable salt of the foregoing, and methods of using the same, in treating, for example, the diseases, disorders, or conditions mediated by the degradation of protein kinases, such as hematopoietic progenitor kinase 1 (HPK1, MAP4K1), mitogen-activated protein kinases 1/2 (MEK 1/2), human Fms-like tyrosine kinase 3 receptor (FLT3), and aurora kinases.

### BACKGROUND OF THE INVENTION

Protein kinases are enzymes that catalyze the phosphorylation of hydroxyl groups on tyrosine, serine, and threonine residues of proteins. Serine/threonine kinases, specific for phosphorylation of serine and threonine residues, constitute an important family of protein kinases. Another major family of protein kinases are tyrosine kinases, specific for phosphorylation of tyrosine residues. In addition, there are dual specificity kinases, which phosphorylate both tyrosine and serine/threonine residues.

Protein kinases play critical roles in many cellular functions, such as proliferation, survival, metabolism, and differentiation. Furthermore, dysregulated protein kinases are disease drivers in many pathological conditions, including immunological, oncological, metabolic, neurological, and infectious diseases. Protein kinases that are involved in cell proliferation and survival are frequently mutated or overexpressed in cancers. They are attractive targets for anticancer drugs.

Hematopoietic progenitor kinase 1 (HPK1, MAP4K1) is a serine/threonine kinase and a member of the MAP4K family. HPK1 is predominantly expressed in hematopoietic cell linages and serves as a negative regulator in T lymphocytes and dendritic cells activation. Therefore, HPK1 inhibition is expected to prolong T cell activation and enhance APC functions by dendritic cells. Thus, HPK1 is identified as a novel anticancer immunotherapy and a new intracellular checkpoint molecule and a potential combination therapy with current checkpoint molecules. Small molecule degraders that target HPK1 can eliminate its scaffolding function to achieve better efficacy and/or overcome resistance to inhibitors.

Mitogen-activated protein kinases 1/2 (MEK 1/2) are dual specificity (threonine &tyrosine) protein kinase that function downstream of RAS in MAP kinase (MAPK) signaling transduction pathway. They are responsible for transmitting growth signal from a variety of extracellular stimuli to downstream effectors ERK1/2. When RAS binds RAF, it phosphorylates and activates MEK1/2. When phosphorylated, MEK1/2 further activate ERK1/2, the only downstream substrates. The MAPK pathway is an important pathway that controls cell proliferation, survival, and differentiation. MEK1/2 inhibitors have been used to treat cancers with overactivated MAPK pathway. 4 MEK inhibitors (MEKis) have been approved by FDA to date, however, their application is limited due to acquired resistance and side effects under long-term treatment. Small molecular degraders that can efficiently eliminate MEK protein are expected to address the limitation of current anti-MEK therapy and bring new breakthrough in cancer treatment.

Human Fms-like tyrosine kinase 3 receptor (FLT3), also known as fetal liver kinase 2 (FLK-2) or CD135, is a member of the receptor tyrosine kinases class III. FLT3 is overexpressed in approximately 90% of acute myeloid leukemia (AML), a majority of acute lymphocytic leukemia (ALL) and the blast-crisis phase of chronic myeloid leukemia (BC-CML). FLT3 is one of the most frequently mutated genes in hematologic malignancies. FLT3 mutations have been found in 1-3% of patients with ALL, 5-10% of patients with myelodysplasia and 15-35% of patients with AML. FLT3 mutations can be subdivided into internal tandem duplicates (ITD), present in approximately 25% of patients, and point mutations (such as D835 and 1836) in the tyrosine kinase domain (TKD), present in approximately 5%. Both FLT3-ITD and FLT3-TKD mutations are constitutively active, leading to ligand-independent FLT3 signaling and cellular proliferation. The current small molecule FLT3 inhibitors did not offer significant clinical benefit as a monotherapy. There is a need for a FLT3 degrader that can induce rapid degradation of FLT3 to efficiently downregulate the downstream STAT5 pathway. Such a Flt3 degrader will provide a new option for patients who have failed all current available drug therapies and stem cell transplantation.

Aurora kinases are key cell cycle regulators implicated in the pathogenesis of several tumor types. In humans, there are three isoforms of Aurora kinases: Aurora A, Aurora B and Aurora C. Aurora A and Aurora B play critical roles in mitotic division, whereas Aurora C activity is largely restricted to meiotic cells. Aurora A and Aurora B are structurally closely related but have distinct roles in mitotic division. The Aurora A gene (AURKA) localizes to chromosome 20ql3.2, which is frequently amplified or overexpressed in a broad array of cancers. The encoded protein is found at the centrosome in interphase cells and at the spindle poles in mitosis. The Aurora A kinase interacts and phosphorylates a diverse set of proteins that collectively function in regulating mitotic progression and cell division. Aurora A is functionally connected to several tumor suppressors and oncogenes. It promotes the transcription of the c-Myc oncogene and protects N-Myc protein from ubiquitination and subsequent degradation. It also downregulates p53 and suppresses the function of BRCA1/2 tumor suppressors. Overexpression of Aurora A kinase can result in a stoichiometric imbalance between Aurora A and its interacting partners, leading to oncogenic transformation. The potential oncogenic role of Aurora A has led to considerable interest in targeting this kinase for the treatment of cancers with genetic instability, aneuploidy, or genetic alterations of oncogenes (e.g. Myc, RAS, PKA) or tumor suppressors (e.g. TP53, BRCA1/2). Despite different Aurora A inhibitors have been tested in clinical trials, limited efficacy and significant toxicity were observed. A novel Aurora A degrader has the potential to improve clinical outcome.

Given the aforementioned importance of protein kinases in tumorigenesis, a small molecule degrader can be used as single agent or in combination to treat solid tumors, including, but not limited to, brain cancer, breast cancer, respiratory tract and/or lung cancer, a reproductive organ cancer, bone cancer, digestive tract cancer, urinary tract cancer, eye cancer, liver cancer, skin cancer, head and neck cancer, anal cancer, nervous system cancer, thyroid cancer, and parathyroid cancer. For example, a small molecule degrader can be used as single agent or in combination to treat hematologic cancers, including, but not limited to, acute myeloid leukemia (AML), acute lymphoblastic leukemia (ALL), multiple myeloma (MM), diffuse large B-cell lymphoma (DLBCL), non-Hodgkin's lymphoma (NHL), Hodgkin's lymphoma (HL), T-cell lymphoma (TCL), Burkitt lymphoma (BL), chronic lymphocytic leukemia/small lymphocytic lymphoma (CLL/SLL), mantle cell lymphoma (MCL), marginal zone lymphoma (MZL), and myelodysplastic syndromes (MDS).

### SUMMARY OF THE INVENTION

One aspect of the present disclosure provides a compound selected from compounds of Formula I, a tautomer thereof, a deuterated derivative of the compound or the tautomer, or a pharmaceutically acceptable salt of the foregoing, which can be employed in the treatment of diseases mediated by the degradation of protein kinases, such as hematopoietic progenitor kinase 1 (HPK1, MAP4K1), mitogen-activated protein kinases 1/2 (MEK 1/2), human Fms-like tyrosine kinase 3 receptor (FLT3), and aurora kinases. For example, disclosed herein is a compound of the following structural Formula I:

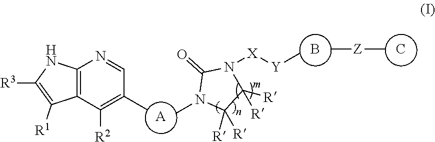

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² and R³ are independently chosen from hydrogen, halogen groups, OR^{x}, SR^{x}, NHR^{x}, N(R^{x})₂, CHR^{x}, and C(R^{x})₂;
(iii) each R′ is independently chosen from hydrogen, halogen groups, linear, branched, and cyclic alkyl groups;
(iv) m and n are independently chosen from 0, 1, and 2;
(v) X is absent or is chosen from linear, branched, cyclic alkylene groups, linear, branched, and cyclic heteroalkylene groups;
(vi) Y and Z are independently absent or chosen from —O—, —C(O)—, —C(O)R^{x}—, —C(S)—, —C(S)R^{x}—, —[C(R^{x}R^{y})]ₚ—, —S(O)₂—, —S(O)₂R^{x}—, NR^{x}—, and —NR^{x}C(O)—, wherein p is chosen from 1, 2, 3, 4, 5, and 6; wherein if X is absent, then Y is not —O—, —S(O)₂—, —S(O)₂R^{x}—, NR^{x}—, or —NR^{x}C(O)—;
(vii) R^{x}, R^{y}, and R^{w} are each independently chosen from hydrogen, linear, branched, and cyclic alkyl groups, carbocyclic groups, heterocyclic groups, aryl groups, and heteroaryl groups;
(viii) ring A is chosen from optionally substituted aryl groups and heteroaryls groups,
(ix) ring B is absent or is chosen from cycloalkyl groups and heterocycloalkyls;
(x) ring C is chosen from

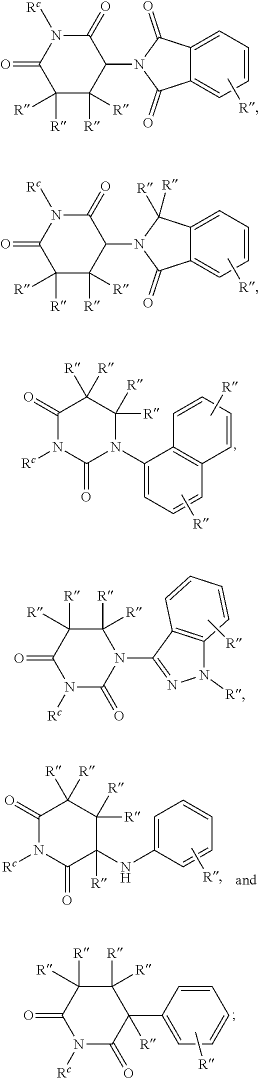

wherein R^{c} is hydrogen; R″ is chosen from hydrogen, halogen groups, OR^{x}, linear,
branched, and cyclic alkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O) C₁-C₆ linear, branched, and cyclic alkyl groups,
—C(O) OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O) C₁-C₆ linear, branched, and cyclic alkyl groups,
—C(O) NHC₁-C₆ linear, branched, and cyclic alkyl groups,
—NHaryl groups,
—N(aryl groups)₂,
—NHC(O) aryl groups,
—C(O) NHaryl groups,
—NHheteroaryl groups,
—N(heteroaryl groups)₂,
—NHC(O) heteroaryl groups,
—C(O) NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

In one aspect of the present disclosure, the compounds of Formula I are selected from Compounds 1 to 18 shown below, a tautomer thereof, a deuterated derivative of the compound or the tautomer, and a pharmaceutically acceptable salt of the foregoing.

In some embodiments, the present disclosure provides pharmaceutical compositions comprising a compound of Formula I, a tautomer thereof, a deuterated derivative of the compound or the tautomer, and/or a pharmaceutically acceptable salt of the foregoing, and a pharmaceutically acceptable carrier. In some embodiments, the pharmaceutical compositions may comprise a compound selected from Compounds 1 to 18 shown below, a tautomer thereof, a deuterated derivative of the compound or the tautomer, and/or a pharmaceutically acceptable salt of the foregoing. These compositions may further comprise an additional active pharmaceutical agent.

Another aspect of the present disclosure provides methods of treating a disease, a disorder, or a condition mediated by the degradation of a protein kinase in a subject, comprising administering a therapeutically effective amount of a compound of Formula I, a tautomer thereof, a deuterated derivative of the compound or the tautomer, and/or a pharmaceutically acceptable salt of the foregoing, or a pharmaceutical composition comprising any of the foregoing. In some embodiments, the methods of treatment comprise administering to a subject, a therapeutically effective amount of a compound selected from Compounds 1 to 18 shown below, a tautomer thereof, a deuterated derivative of the compound or the tautomer, and/or a pharmaceutically acceptable salt of the foregoing, or a pharmaceutical composition comprising any of the foregoing.

In some embodiments disclosed herein, the methods of treatment comprise administration of an additional active pharmaceutical agent to the subject in need thereof, either in the same pharmaceutical composition as a compound of Formula I, a tautomer thereof, a deuterated derivative of the compound or the tautomer, and/or a pharmaceutically acceptable salt of the foregoing, or in a separate composition. In some embodiments disclosed herein, the methods of treatment comprise administering a compound selected from Compounds 1 to 18 shown below, a tautomer thereof, a deuterated derivative of the compound or the tautomer, and/or a pharmaceutically acceptable salt of the foregoing with an additional active pharmaceutical agent either in the same composition or in a separate composition.

Also disclosed herein are methods of decreasing protein kinase activity, comprising administering to a subject a therapeutically effective amount of a compound of Formula I, a tautomer thereof, a deuterated derivative of the compound or the tautomer, and/or a pharmaceutically acceptable salt of the foregoing, or a pharmaceutical composition comprising any of the foregoing. In some embodiments disclosed herein, the methods of degrading a protein kinase comprise administering to a subject, a compound selected from Compounds 1 to 18 shown below, a tautomer thereof, a deuterated derivative of the compound or the tautomer, and/or a pharmaceutically acceptable salt of the foregoing, or a pharmaceutical composition comprising any of the foregoing.

### BRIEF DESCRIPTION OF THE FIGURES

The foregoing summary, as well as the following detailed description of the disclosure, will be better understood when read in conjunction with the appended drawings. For the purpose of illustrating the present disclosure, the appended drawings illustrate some, but not all, alternative embodiments. It should be understood, however, that the disclosure is not limited to the precise arrangements and instrumentalities shown. These drawings, which are incorporated into and constitute part of the specification, assist in explaining the principles of the disclosure.

FIG. 1 shows a Western blot of the degradation of HPK1, FLT3, and MEK 1/2 by Example 2 of the present disclosure.

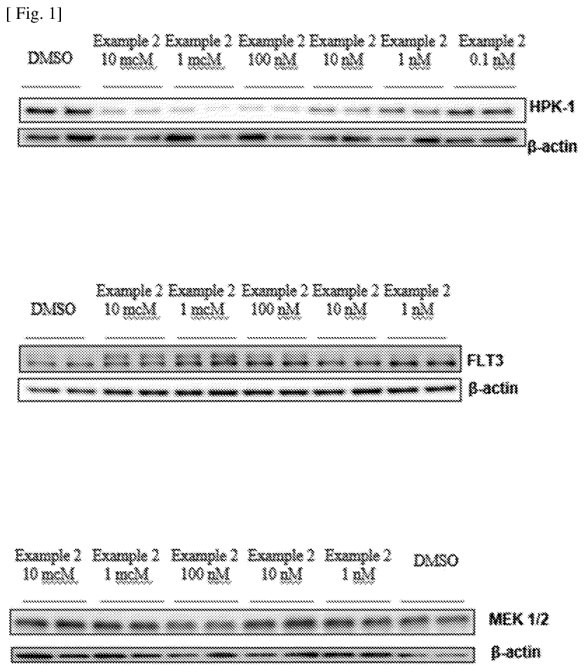

FIG. 2 shows plotted data of the degradation of HPK1, FLT3, and MEK 1/2 by Example 2 of the present disclosure.

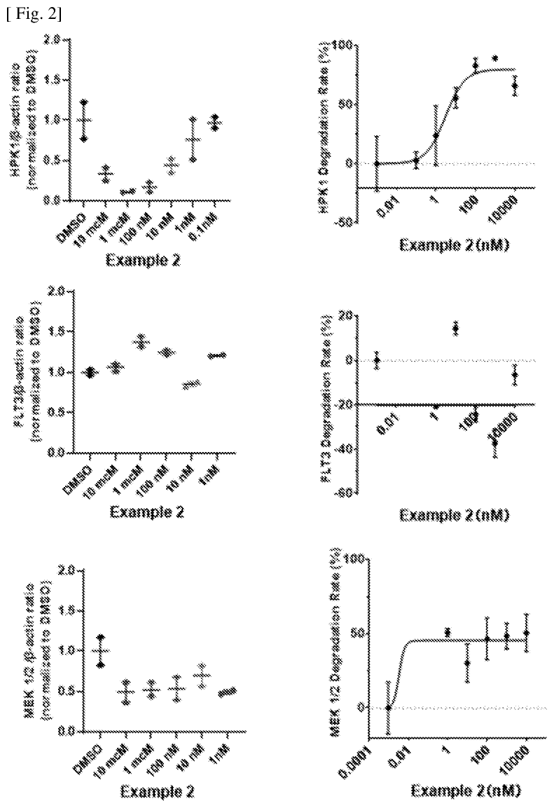

FIG. 3 shows a Western blot of the degradation of HPK1, FLT3, Aurora A, and MEK 1/2 by Example 8 of the present disclosure.

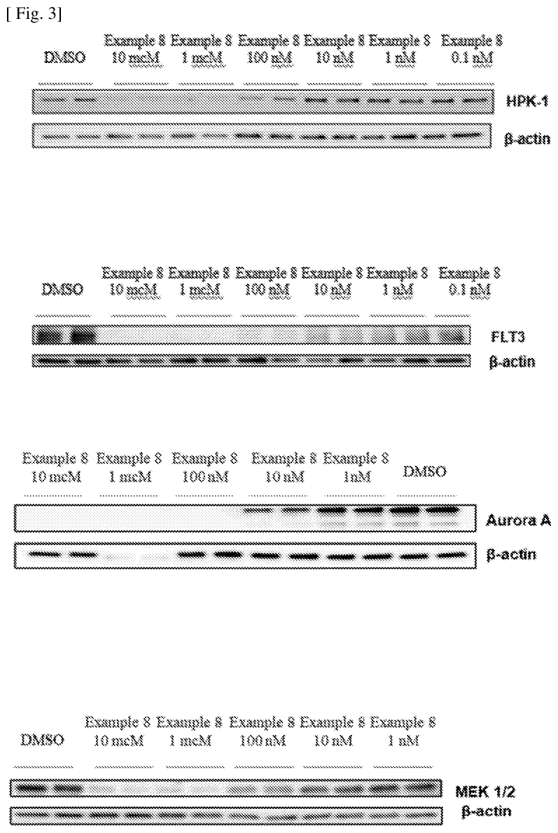

FIG. 4 shows plotted data of the degradation of HPK1 and FLT3 by Example 8 of the present disclosure.

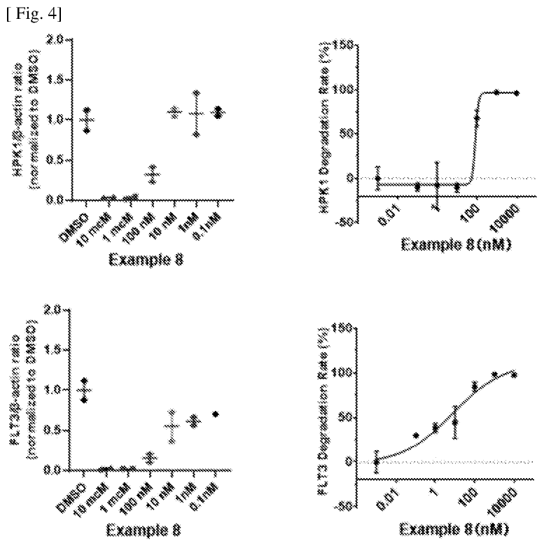

FIG. 5 shows plotted data of the degradation of Aurora A, and MEK 1/2 by Example 8 of the present disclosure.

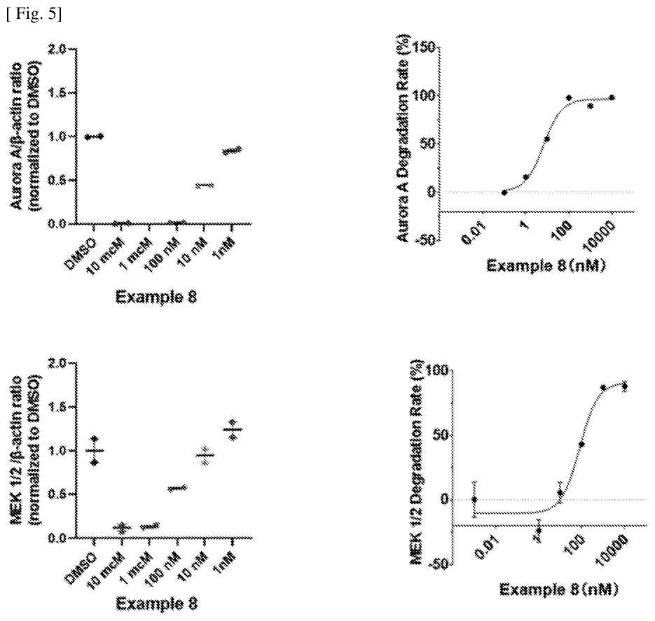

FIG. 6 shows a Western blot of the degradation of FLT3 and Aurora A by Example 14 of the present disclosure.

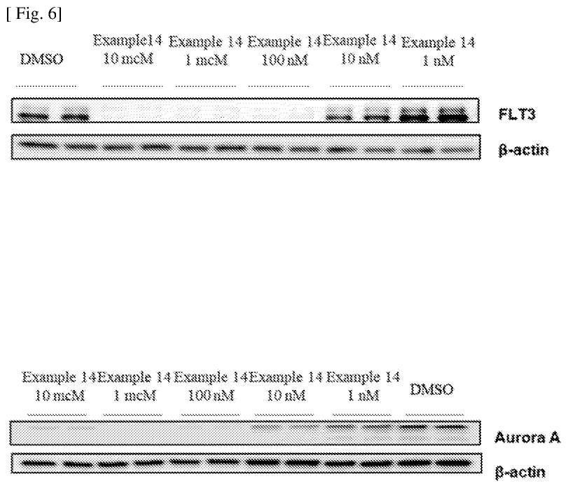

FIG. 7 shows plotted data of the degradation of FLT3 and Aurora A by Example 14 of the present disclosure.

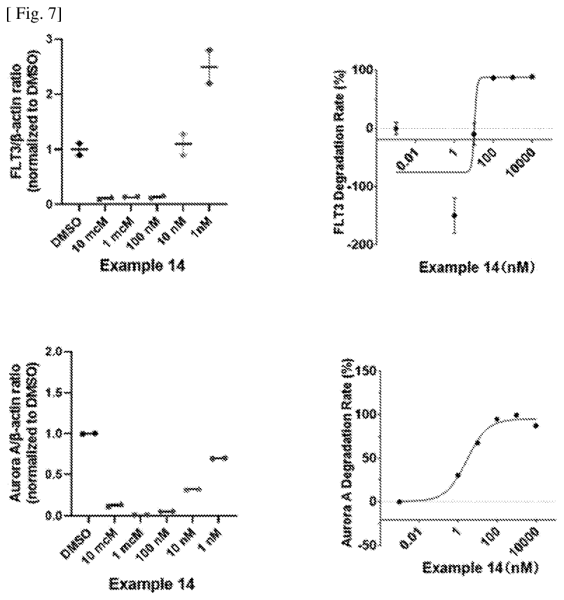

### DETAILED DESCRIPTION

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “protein kinase” is an enzyme that catalyzes the phosphorylation of hydroxyl groups on tyrosine, serine, and threonine residues of proteins. Serine/threonine kinases, specific for phosphorylation of serine and threonine residues, constitute an important family of protein kinases. Another major family of protein kinases are tyrosine kinases, specific for phosphorylation of tyrosine residues. In addition, there are dual specificity kinases, which phosphorylate both tyrosine and serine/threonine residues. Examples of protein kinases include but are not limited to hematopoietic progenitor kinases, mitogen-activated protein kinases 1/2, Human Fms-like tyrosine kinase 3, and Aurora kinases.

The term “HPK1” or “hematopoietic progenitor kinase 1” as used herein, also known as MAP4K1, is a serine/threonine kinase and is predominantly expressed in hematopoietic cells, such as T cells, B cells and dendritic cells (DC). HPK1 is involved in the modulation of various downstream signaling pathways, such as extracellular signal-regulated kinase (ERK), c-Jun N-terminal kinase (JNK), and nuclear factor-κB (NF-κB), which are all associated with the regulation of cellular proliferation and immune cell activation.

The term “MEK 1/2” or “mitogen-activated protein kinases 1/2” as used herein are dual specificity (threonine &tyrosine) protein kinase that function downstream of RAS in MAP kinase (MAPK) signaling transduction pathway. They are responsible for transmitting growth signal from a variety of extracellular stimuli to downstream effectors ERK1/2. When RAS binds RAF, it phosphorylates and activates MEK1/2. When phosphorylated, MEK1/2 further activate ERK1/2, the only downstream substrates. The MAPK pathway is an important pathway that controls cell proliferation, survival, and differentiation. MEK1/2 inhibitors have been used to treat cancers with overactivated MAPK pathway.

The term “FLT3” or “Human Fms-like tyrosine kinase 3 receptor” as used herein, also known as fetal liver kinase 2 (FLK-2) or CD135, is a member of the receptor tyrosine kinases class III. FLT3 is overexpressed in approximately 90% of acute myeloid leukemia (AML), a majority of acute lymphocytic leukemia (ALL) and the blast-crisis phase of chronic myeloid leukemia (BC-CML). FLT3 is one of the most frequently mutated genes in hematologic malignancies. FLT3 mutations have been found in 1-3% of patients with ALL, 5-10% of patients with myelodysplasia and 15-35% of patients with AML. FLT3 mutations can be subdivided into internal tandem duplicates (ITD), present in approximately 25% of patients, and point mutations (such as D835 and 1836) in the tyrosine kinase domain (TKD), present in approximately 5%. Both FLT3-ITD and FLT3-TKD mutations are constitutively active, leading to ligand-independent FLT3 signaling and cellular proliferation.

The term “Aurora kinase” as used herein is a key cell cycle regulator implicated in the pathogenesis of several tumor types. In humans, there are three isoforms of Aurora kinases: Aurora A, Aurora B and Aurora C. Aurora A and Aurora B play critical roles in mitotic division, whereas Aurora C activity is largely restricted to meiotic cells. Aurora A and Aurora B are structurally closely related but have distinct roles in mitotic division. The Aurora A gene (AURKA) localizes to chromosome 20ql3.2, which is frequently amplified or overexpressed in a broad array of cancers. The encoded protein is found at the centrosome in interphase cells and at the spindle poles in mitosis. The Aurora A kinase interacts and phosphorylates a diverse set of proteins that collectively function in regulating mitotic progression and cell division. Aurora A is functionally connected to several tumor suppressors and oncogenes. It promotes the transcription of the c-Myc oncogene and protects N-Myc protein from ubiquitination and subsequent degradation. It also downregulates p53 and suppresses the function of BRCA1/2 tumor suppressors. Overexpression of Aurora A kinase can result in a stoichiometric imbalance between Aurora A and its interacting partners, leading to oncogenic transformation. The potential oncogenic role of Aurora A has led to considerable interest in targeting this kinase for the treatment of cancers with genetic instability, aneuploidy, or genetic alterations of oncogenes (e.g. Myc, RAS, PKA) or tumor suppressors (e.g. TP53, BRCA1/2).

Compounds disclosed herein can degrade protein kinases. Thus, compounds disclosed herein are generally useful in the treatment of diseases or conditions associated with such kinases. In one embodiment, the compounds disclosed herein are HPK1 degraders, MEK 1/2 degraders, FLT3 degraders, or Aurora A degraders, and are useful for treating diseases, such as cancer, associated with such kinases.

The term “degrader” as used herein, refers to a molecule agent that binds to a protein kinase, such as hematopoietic progenitor kinase 1 and subsequently lowers the steady state protein levels of the kinase. In some embodiments, a degrader as disclosed herein lowers steady state protein kinase levels by at least 10%, at least 20%, at least 30%, at least 40%, at least 50%, at least 60%, at least 65%, at least 70%, at least 75%, at least 80%, at least 85%, at least 90%, at least 95%, at least 96%, at least 97%, at least 98%, or at least 99%. In some embodiments, a degrader as disclosed herein lowers steady state protein kinase levels by at least 65%. In some embodiments, a degrader as disclosed herein lowers steady state protein kinase levels by at least 85%.

The term “compound,” when referring to a compound of the present disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of the present disclosure will depend upon a number of factors, including, for example, the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by the present disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of the present disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the present disclosure. Unless otherwise stated, all tautomeric forms of the compounds of the present disclosure are within the scope of the present disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives disclosed herein. Thus, unless otherwise stated, when a reference is made to a “deuterated derivative” of a compound of the present disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium).

The term “isotopic enrichment factor” as used herein means the ratio between the isotopic abundance and the natural abundance of a specified isotope.

The term “alkyl” as used herein, means a linear or branched, substituted or unsubstituted hydrocarbon chain that is completely saturated. Unless otherwise specified, an alkyl group contains 1 to 30 alkyl carbon atoms. In some embodiments, an alkyl group contains 1 to 20 alkyl carbon atoms. In some embodiments, an alkyl group contains 1 to 10 aliphatic carbon atoms. In some embodiments, an alkyl group contains 1 to 8 aliphatic carbon atoms. In some embodiments, an alkyl group contains 1 to 6 alkyl carbon atoms. In some embodiments, an alkyl group contains 1 to 4 alkyl carbon atoms. In other embodiments, an alkyl group contains 1 to 3 alkyl carbon atoms. And in yet other embodiments, an alkyl group contains 1 to 2 alkyl carbon atoms. In some embodiments, alkyl groups are substituted. In some embodiments, alkyl groups are unsubstituted. In some embodiments, alkyl groups are linear or straight-chain or unbranched. In some embodiments, alkyl groups are branched.

The term “cycloalkyl” refers to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, cycloalkyl groups are substituted. In some embodiments, cycloalkyl groups are unsubstituted. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentyl, and cyclohexyl.

The term “carbocyclyl” encompasses the term “cycloalkyl” and refers to a monocyclic C₃₋₈ hydrocarbon or a spirocyclic, fused, or bridged bicyclic or tricyclic C₈₋₁₄ hydrocarbon that is completely saturated, or is partially saturated as it contains one or more units of unsaturation but is not aromatic, wherein any individual ring in said bicyclic ring system has 3 to 7 members. Bicyclic carbocyclyls include combinations of a monocyclic carbocyclic ring fused to, for example, a phenyl. In some embodiments, carbocyclyl groups are substituted. In some embodiments, carbocyclyl groups are unsubstituted. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl. Non-limiting examples of monocyclic carbocyclyls include cyclopropyl, cyclobutyl, cyclopentanyl, cyclohexyl, cyclopentenyl, cyclohexenyl, etc.

The term “alkylene” as used herein, refers to a divalent alkyl radical. Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2, 2-dimethylpentylene, 2, 3-dimethylpentylene, n-heptylene, n-octylene, n-nonylene and n-decylene.

The term “alkenyl” as used herein, means a linear or branched, substituted or unsubstituted hydrocarbon chain that contains one or more double bonds. In some embodiments, alkenyl groups are substituted. In some embodiments, alkenyl groups are unsubstituted. In some embodiments, alkenyl groups are linear, straight-chain, or unbranched. In some embodiments, alkenyl groups are branched.

The term “alkynyl” as used herein, refers to an unsaturated straight or branched hydrocarbon having at least one carbon-carbon triple bond, such as a straight or branched group of 2 to 8 carbon atoms, referred to herein as C₂₋₈alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “heterocyclyl” as used herein means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include, for example, the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl. In some embodiments, the “heterocyclyl” group contains 3 to 14 ring members in which one or more ring members is a heteroatom independently chosen, for example, from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted. In some embodiments, the heterocyclyl is a 3- to 12-membered heterocyclyl. In some embodiments, the heterocyclyl is a 4- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. In some embodiments, the heterocyclyl is a 6-membered heterocyclyl. Non-limiting examples of monocyclic heterocyclyls include piperidinyl, piperazinyl, morpholinyl, tetrahydropyranyl, azetidinyl, oxetanyl, tetrahydrothiophenyl, dihydropyranyl, tetrahydropyridinyl, etc.

The term “heteroatom” means one or more of oxygen, sulfur, and nitrogen, including, any oxidized form of nitrogen or sulfur, or silicon; the quaternized form of any basic nitrogen or; a substitutable nitrogen of a heterocyclic ring, for example N (as in 3, 4-dihydro-2H-pyrrolyl), NH (as in pyrrolidinyl) or NR⁺ (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains double or triple bonds.

The term “alkoxy” as used herein, refers to an alkyl group, as defined above, wherein one carbon of the alkyl group is replaced by an oxygen (“alkoxy”) atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refer to —C═N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Nonlimiting examples of aromatic rings include aryl and heteroaryl rings that are further defined as follows.

The term “aryl” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Nonlimiting examples of aryl groups include phenyl (C₆) and naphthyl (C₁₀) rings. In some embodiments, aryl groups are substituted. In some embodiments, aryl groups are unsubstituted.

The term “heteroaryl” refers to monocyclic or spirocyclic, fused, or bridged bicyclic or tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. Bicyclic heteroaryls include, for example, the following combinations of monocyclic rings: a monocyclic heteroaryl fused to another monocyclic heteroaryl; and a monocyclic heteroaryl fused to a phenyl. In some embodiments, heteroaryl groups are substituted. In some embodiments, heteroaryl groups have one or more heteroatoms chosen, for example, from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, etc.

A “spirocyclic ring system” refers to a ring system having two or more cyclic rings, where every two rings share only one common atom.

The term “pro-drug group” refers to a group that is covalently attached to a compound and results in a compound with improved oral bioavailability and/or tumor targeting and/or that is more active in vivo. Certain compounds of Formula I may include a pro-drug group, as described in Hydrolysis in Drug and Prodrug Metabolism: Chemistry, Biochemistry, and Enzymology (see Testa, Bernard and Mayer, Joachim M. Wiley-VHCA, Zurich, Switzerland 2003). Pro-drugs of the compounds described herein are structurally modified forms of the compound that readily undergo chemical changes under physiological conditions to provide the active compound. Pro-drugs are often useful because, in some situations, they may be easier to administer than the parent drug. They may, for instance, be bioavailable by oral administration whereas the parent drug is not. A wide variety of pro-drug derivatives are known in the art, such as those that rely on hydrolytic cleavage or oxidative activation of the pro-drug. An example, without limitation, of a pro-drug group would be a portion of a compound such as an ester, but then is metabolically hydrolyzed to the carboxylic acid to release the active entity. Additional examples of pro-drug groups include peptidyl derivatives of a compound.

Non-limiting examples of suitable solvents that may be used in the present disclosure include water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1, 4-dioxane, and N-methyl pyrrolidone (NMP).

Non-limiting examples of suitable bases that may be used in the present disclosure include 1, 8-diazabicyclo [5.4.0] undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

Disclosed herein are pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of the present disclosure. Suitable pharmaceutically acceptable salts are, for example, those disclosed in S. M. Berge, et al. J. Pharmaceutical Sciences, 1977, 66, pp. 1-19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1, 4-dioate, hexyne-1, 6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. The present disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The term “subject” refers to an animal, including but not limited to, a human.

The term “therapeutically effective amount” refers to that amount of a compound that produces the desired effect for which it is administered (e.g., improvement in symptoms of diseases, disorders, and conditions mediated by the degradation of HPK1, lessening the severity of diseases, disorders, and conditions mediated by the degradation of HPK1 or a symptom thereof, and/or reducing progression of diseases, disorders, and conditions mediated by the degradation of HPK1 or a symptom thereof). The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, lower risk of diseases, disorders, and conditions mediated by the degradation of HPK1, and disease-related complications. Improvements in or lessening the severity of any of these symptoms can be readily assessed according to methods and techniques known in the art or subsequently developed.

The term “cancer” includes, but is not limited to, the following cancers: epidermoid oral such as buccal cavity, lip, tongue, mouth, pharynx; cardiac cancers such as sarcoma (angiosarcoma, fibrosarcoma, rhabdomyosarcoma, liposarcoma), myxoma, rhabdomyoma, fibroma, lipoma, and teratoma; lung cancers such as bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatosis hamartoma, mesothelioma; gastrointestinal cancers such as esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Kaposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers such as hepatoma (hepatocellular carcinoma), cholangiocarcinoma, hepatoblastoma, angiosarcoma, hepatocellular adenoma, hemangioma, biliary passages; bone cancers such as osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; cancers of the nervous system, including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosathecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers such as blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma) hairy cell; lymphoid disorders; skin cancers including malignant melanoma, basal cell carcinoma, squamous cell carcinoma, Kaposi's sarcoma, keratoacanthoma, moles dysplastic nevi, lipoma, angioma, dermatofibroma, keloids, psoriasis; cancers of the thyroid gland such as papillary thyroid carcinoma, follicular thyroid carcinoma; medullary' thyroid carcinoma, undifferentiated thyroid cancer, multiple endocrine neoplasia type 2A, multiple endocrine neoplasia type 2B, familial medullary thyroid cancer, pheochromocytoma, paraganglioma; and cancers of the adrenal glands like neuroblastoma.

Compounds and compositions of the application can be administered in therapeutically effective amounts in a combinational therapy with one or more therapeutic agents (pharmaceutical combinations) or modalities, e.g., anti-proliferative, anti-cancer, immunomodulatory or anti-inflammatory agent, and/or non-drug therapies, etc. For example, synergistic effects can occur with anti-proliferative, anti-cancer, immunomodulatory or anti-inflammatory substances. Where the compounds of the application are administered in conjunction with other therapies, dosages of the co-administered compounds will of course vary depending on the type of co-drug employed, on the specific drug employed, on the condition being treated and so forth. Combination therapy includes the administration of the subject compounds in further combination with one or more other biologically active ingredients (such as, but not limited to, a second kinase inhibitor, a second and different antineoplastic agent, and non-drug therapies (such as, but not limited to, surgery or radiation treatment). For instance, the compounds of the application can be used in combination with other pharmaceutically active compounds, preferably compounds that are able to enhance the effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy. In another aspect of the application, the compounds may be administered in combination with one or more separate pharmaceutical agents, e.g., a chemotherapeutic agent, an immunotherapeutic agent, or an adjunctive therapeutic agent. In an embodiment, the separate pharmaceutical agent is selected from the group consisting of an anti-PD1 antibody (e.g. pembrolizumab), an HDAC inhibitor r (e.g. panobinostat, romidepsin, vorinostat, or citarinostat), a BCL-2 inhibitor (e.g. venetoclax), a BTK inhibitor (e.g. ibrutinib or acalabrutinib), an mTOR inhibitor (e.g. everolimus), a PI3K inhibitor r (e.g. idelalisib), a PKCβ inhibitor (e.g. enzastaurin), a SYK inhibitor (e.g. fostamatinib), a JAK2 inhibitor (e.g. fedratinib, pacritinib, ruxolitinib, baricitinib, gandotinib, lestaurtinib, or momelotinib), an Aurora kinase inhibitor (e.g. alisertib), an EZF12 inhibitor (e.g. tazemetostat, GSK126, CPI-1205, 3-deazaneplanocin A, EPZ005687, Ell, UNC1999, or sinefungin), a BET inhibitor (e.g. birabresib), a hypomethylating agent (e.g. 5-azacytidine or decitabine), a DOTlL inhibitor (e.g. pinometostat), a FIAT inhibitor (e.g. C646), a WDR5 inhibitor (e.g. OICR-9429), a DNMTl inhibitor (e.g. GSK3484862), an LSD-1 inhibitor (e.g. Compound C or seclidemstat), a G9A inhibitor (e.g. UNC0631), a PRMT5 inhibitor (e.g. GSK3326595), a BRD inhibitor (e.g. LP99), a SUV420FU/F12 inhibitor (e.g. A-196), a CARM1 inhibitor (e.g. EZM2302), a PLK1 inhibitor (e.g. B12536), an NEK2 inhibitor (e.g. JF1295), an MEK inhibitor (e.g. trametinib, binimetinib, cobimetinib, selumetinib), a PF1F19 inhibitor, a PIM inhibitor (e.g. LGF1-447), an IGF-IR inhibitor (e.g. linsitinib), an XPOl inhibitor (e.g. selinexor), a BIRC5 inhibitor (e.g. YMl 55), a PARP inhibitor (e.g. Olaparib), an EGFR inhibitor (e.g. Osimertinib), a HER2/NEU inhibitor (i.e. tucatinib), an SRC inhibitor (i.e. dasatinib), an AKT inhibitor (i.e. Ipatasertib), a KRAS (G12C) inhibitor (i.e. Sotorasib), a KRAS (G12D) inhibitor (i.e. MRTX1133), platinum, or a chemotherapy (e.g, bendamustine, bleomycin, doxorubicin, etoposide, methotrexate, cytarabine, vincristine, ifosfamide, melphalan, oxaliplatin, cisplatin, taxanes or dexamethasone).

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

### II. Compounds and Compositions

In a first embodiment, a compound of the present disclosure is a compound of the following structural formula I:

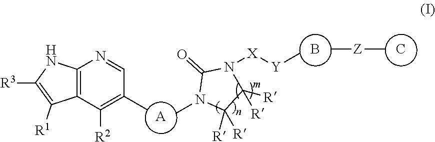

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² and R³ are independently chosen from hydrogen, halogen groups, OR^{x}, SR^{x}, NHR^{x}, N(R^{x})₂, CHR^{x}, and C(R^{x})₂;
(iii) each R is independently chosen from hydrogen, halogen groups, linear, branched, and cyclic alkyl groups;
(iv) m and n are independently chosen from 0, 1, and 2;
(v) X is absent or is chosen from linear, branched, cyclic alkylene groups, linear, branched, and cyclic heteroalkylene groups;
(vi) Y and Z are independently absent or chosen from —O—, —C(O)—, —C(O)R^{x}—, —C(S)—, —C(S)R^{x}—, —[C(R^{x}R^{y})]ₚ—, —S(O)₂—, —S(O)₂R^{x}—, NR^{x}—, and —NR^{x}C(O)—, wherein p is chosen from 1, 2, 3, 4, 5, and 6; wherein if X is absent, then Y is not —O—, —S(O)₂—, —S(O)₂R^{x}—, NR^{x}—, or —NR^{x}C(O)—;
(vii) R^{x}, R^{y}, and R^{w} are each independently chosen from hydrogen, linear, branched, and cyclic alkyl groups, carbocyclic groups, heterocyclic groups, aryl groups, and heteroaryl groups;
(viii) ring A is chosen from optionally substituted aryl groups and heteroaryls groups,
(ix) ring B is absent or is chosen from cycloalkyl groups and heterocycloalkyls;
(x) ring C is chosen from

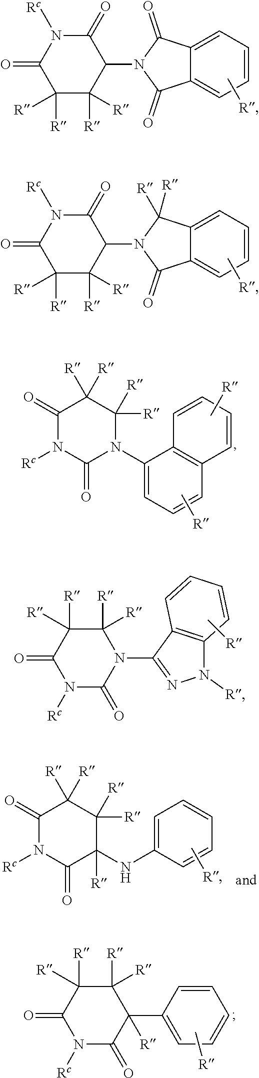

wherein R^{c} is hydrogen; R″ is chosen from hydrogen, halogen groups, OR^{x}, linear,
branched, and cyclic alkyl groups; wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O) C₁-C₆ linear, branched, and cyclic alkyl groups,
—C(O) OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O) C₁-C₆ linear, branched, and cyclic alkyl groups,
—C(O) NHC₁-C₆ linear, branched, and cyclic alkyl groups,
—NHaryl groups,
—N(aryl groups)₂,
—NHC(O) aryl groups,
—C(O) NHaryl groups,
—NHheteroaryl groups,
—N(heteroaryl groups)₂,
—NHC(O) heteroaryl groups,
—C(O) NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

In a second embodiment, in a compound, tautomer, deuterated derivative, or pharmaceutically acceptable salt of the present disclosure, R¹ is chosen from linear, branched, and cyclic alkyl groups; R² is a halogen group; and R³ is chosen from hydrogen, linear, branched, and cyclic alkyl groups; and all other variables not specifically defined herein are as defined in the first embodiment.

In a third embodiment, in a compound, tautomer, deuterated derivative, or pharmaceutically acceptable salt of the present disclosure, R¹ is chosen from C₁-C₆ linear, branched, and cyclic alkyl groups; and all other variables not specifically defined herein are as defined in the first or second embodiment.

In a fourth embodiment, in a compound, tautomer, deuterated derivative, or pharmaceutically acceptable salt of the present disclosure, R¹ is chosen from methyl, ethyl, cyclopropyl, and cyclobutyl; and all other variables not specifically defined herein are as defined in the third embodiment.

In a fifth embodiment, in a compound, tautomer, deuterated derivative, or pharmaceutically acceptable salt of the present disclosure, R² is a halogen group; and all other variables not specifically defined herein are as defined in the proceeding embodiments.

In a sixth embodiment, in a compound, tautomer, deuterated derivative, or pharmaceutically acceptable salt of the present disclosure, R² is chloro; and all other variables not specifically defined herein are as defined in the fifth embodiment.

In a seventh embodiment, in a compound, tautomer, deuterated derivative, or pharmaceutically acceptable salt of the present disclosure, R² is hydrogen; and all other variables not specifically defined herein are as defined in any one of the first to the fourth embodiment.

In an eighth embodiment, in a compound, tautomer, deuterated derivative, or pharmaceutically acceptable salt of the present disclosure, R³ is a halogen group; and all other variables not specifically defined herein are as defined in the proceeding embodiments.

In a ninth embodiment, in a compound, tautomer, deuterated derivative, or pharmaceutically acceptable salt of the present disclosure, R³ is chloro; and all other variables not specifically defined herein are as defined in the eighth embodiment.

In a tenth embodiment, in a compound, tautomer, deuterated derivative, or pharmaceutically acceptable salt of the present disclosure, R³ is hydrogen; and all other variables not specifically defined herein are as defined in any one of the first to the seventh embodiments.

In an eleventh embodiment, in a compound, tautomer, deuterated derivative, or pharmaceutically acceptable salt of the present disclosure, m is 1 and n is 1; and all other variables not specifically defined herein are as defined in the proceeding embodiments.

In a twelfth embodiment, in a compound, tautomer, deuterated derivative, or pharmaceutically acceptable salt of the present disclosure, R′ is hydrogen; and all other variables not specifically defined herein are as defined in the eleventh embodiment.

In a thirteenth embodiment, in a compound, tautomer, deuterated derivative, or pharmaceutically acceptable salt of the present disclosure, m is 2 and n is 1; and all other variables not specifically defined herein are as defined in the proceeding embodiments.

In a fourteenth embodiment, in a compound, tautomer, deuterated derivative, or pharmaceutically acceptable salt of the present disclosure, R′ is hydrogen; and all other variables not specifically defined herein are as defined in the thirteenth embodiment.

In a fifteenth embodiment, in a compound, tautomer, deuterated derivative, or pharmaceutically acceptable salt of the present disclosure, X is absent; and all other variables not specifically defined herein are as defined in the proceeding embodiments.

In a sixteenth embodiment, in a compound, tautomer, deuterated derivative, or pharmaceutically acceptable salt of the present disclosure, X is a linear alkylene group; and all other variables not specifically defined herein are as defined in the proceeding embodiments.

In a seventeenth embodiment, in a compound, tautomer, deuterated derivative, or pharmaceutically acceptable salt of the present disclosure, X is a methylene group; and all other variables not specifically defined herein are as defined in the sixteenth embodiment.

In an eighteenth embodiment, in a compound, tautomer, deuterated derivative, or pharmaceutically acceptable salt of the present disclosure, X is an ethylene group; and all other variables not specifically defined herein are as defined in the sixteenth embodiment.

In a nineteenth embodiment, in a compound, tautomer, deuterated derivative, or pharmaceutically acceptable salt of the present disclosure, wherein Y is absent; and all other variables not specifically defined herein are as defined in the proceeding embodiments.

In a twentieth embodiment, in a compound, tautomer, deuterated derivative, or pharmaceutically acceptable salt of the present disclosure, ring B is chosen from optionally substituted heterocycloalkyls; and all other variables not specifically defined herein are as defined in the proceeding embodiments.

In a twenty-first embodiment, in a compound, tautomer, deuterated derivative, or pharmaceutically acceptable salt of the present disclosure, ring B is chosen from

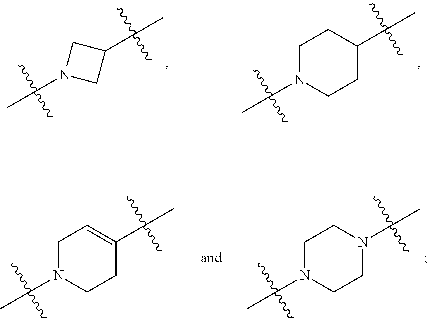 [SMILES: *C1CN(*)C1 ; *C1CCN(*)CC1 ; *C1=CCN(*)CC1 ; *N1CCN(*)CC1]

and all other variables not specifically defined herein are as defined in the twentieth embodiment.

In a twenty-second embodiment, in a compound, tautomer, deuterated derivative, or pharmaceutically acceptable salt of the present disclosure, wherein Z is absent; and all other variables not specifically defined herein are as defined in the proceeding embodiments.

In a twenty-third embodiment, in a compound, tautomer, deuterated derivative, or pharmaceutically acceptable salt of the present disclosure, ring C is

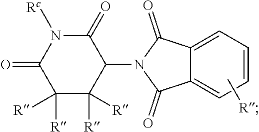

and all other variables not specifically defined herein are as defined in the proceeding embodiments.

In a twenty-fourth embodiment, in a compound, tautomer, deuterated derivative, or pharmaceutically acceptable salt of the present disclosure, R^{c} is hydrogen; and all other variables not specifically defined herein are as defined in the twenty-third embodiment.

In a twenty-fifth embodiment, in a compound, tautomer, deuterated derivative, or pharmaceutically acceptable salt of the present disclosure, R^{c} is chosen from linear, branched, and cyclic alkyl groups; and all other variables not specifically defined herein are as defined in the twenty-third embodiment.

In a twenty-sixth embodiment, in a compound, tautomer, deuterated derivative, or pharmaceutically acceptable salt of the present disclosure, R^{c} is a pro-drug group; and all other variables not specifically defined herein are as defined in the twenty-third embodiment.

In a twenty-seventh embodiment, in a compound, tautomer, deuterated derivative, or pharmaceutically acceptable salt of the present disclosure, ring C is

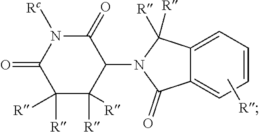

and all other variables not specifically defined herein are as defined in any of embodiments 1-22.

In a twenty-eighth embodiment, in a compound, tautomer, deuterated derivative, or pharmaceutically acceptable salt of the present disclosure, R^{c} is hydrogen; and all other variables not specifically defined herein are as defined in the twenty-seventh embodiment.

In a twenty-ninth embodiment, in a compound, tautomer, deuterated derivative, or pharmaceutically acceptable salt of the present disclosure, R^{c} is chosen from linear, branched, and cyclic alkyl groups; and all other variables not specifically defined herein are as defined in the twenty-seventh embodiment.

In a thirtieth embodiment, in a compound, tautomer, deuterated derivative, or pharmaceutically acceptable salt of the present disclosure, R^{c} is a pro-drug group; and all other variables not specifically defined herein are as defined in the twenty-seventh embodiment.

In a thirty-first embodiment, in a compound, tautomer, deuterated derivative, or pharmaceutically acceptable salt of the present disclosure, ring C is

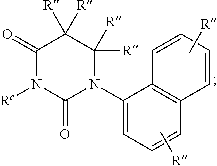

and all other variables not specifically defined herein are as defined in any of embodiments 1-22.

In a thirty-second embodiment, in a compound, tautomer, deuterated derivative, or pharmaceutically acceptable salt of the present disclosure, R^{c} is hydrogen; and all other variables not specifically defined herein are as defined in the thirty-first embodiment.

In a thirty-third embodiment, in a compound, tautomer, deuterated derivative, or pharmaceutically acceptable salt of the present disclosure, R^{c} is chosen from linear, branched, and cyclic alkyl groups; and all other variables not specifically defined herein are as defined in the thirty-first embodiment.

In a thirty-fourth embodiment, in a compound, tautomer, deuterated derivative, or pharmaceutically acceptable salt of the present disclosure, R^{c} is a pro-drug group; and all other variables not specifically defined herein are as defined in the thirty-first embodiment.

In a thirty-fifth embodiment, in a compound, tautomer, deuterated derivative, or pharmaceutically acceptable salt of the present disclosure, ring C is

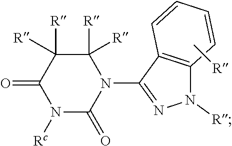

and all other variables not specifically defined herein are as defined in any of embodiments 1-22.

In a thirty-sixth embodiment, in a compound, tautomer, deuterated derivative, or pharmaceutically acceptable salt of the present disclosure, R^{c} is hydrogen; and all other variables not specifically defined herein are as defined in the thirty-fifth embodiment.

In a thirty-seventh embodiment, in a compound, tautomer, deuterated derivative, or pharmaceutically acceptable salt of the present disclosure, R^{c} is chosen from linear, branched, and cyclic alkyl groups; and all other variables not specifically defined herein are as defined in the thirty-fifth embodiment.

In a thirty-eighth embodiment, in a compound, tautomer, deuterated derivative, or pharmaceutically acceptable salt of the present disclosure, R^{c} is a pro-drug group; and all other variables not specifically defined herein are as defined in the thirty-fifth embodiment.

In a thirty-ninth embodiment, in a compound, tautomer, deuterated derivative, or pharmaceutically acceptable salt of the present disclosure, ring C is

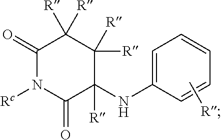

and all other variables not specifically defined herein are as defined in any of embodiments 1-22.

In a fortieth embodiment, in a compound, tautomer, deuterated derivative, or pharmaceutically acceptable salt of the present disclosure, R^{c} is hydrogen; and all other variables not specifically defined herein are as defined in the thirty-ninth embodiment.

In a forty-first embodiment, in a compound, tautomer, deuterated derivative, or pharmaceutically acceptable salt of the present disclosure, R^{c} is chosen from linear, branched, and cyclic alkyl groups; and all other variables not specifically defined herein are as defined in the thirty-ninth embodiment.

In a forty-second embodiment, in a compound, tautomer, deuterated derivative, or pharmaceutically acceptable salt of the present disclosure, R^{c} is a pro-drug group; and all other variables not specifically defined herein are as defined in the thirty-ninth embodiment.

In a forty-third embodiment, in a compound, tautomer, deuterated derivative, or pharmaceutically acceptable salt of the present disclosure, ring C is

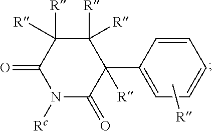

and all other variables not specifically defined herein are as defined in any of embodiments 1-22.

In a forty-fourth embodiment, in a compound, tautomer, deuterated derivative, or pharmaceutically acceptable salt of the present disclosure, R^{c} is hydrogen; and all other variables not specifically defined herein are as defined in the forty-third embodiment.

In a forty-fifth embodiment, in a compound, tautomer, deuterated derivative, or pharmaceutically acceptable salt of the present disclosure, R^{c} is chosen from linear, branched, and cyclic alkyl groups; and all other variables not specifically defined herein are as defined in the forty-third embodiment.

In a forty-sixth embodiment, in a compound, tautomer, deuterated derivative, or pharmaceutically acceptable salt of the present disclosure, R^{c} is a pro-drug group; and all other variables not specifically defined herein are as defined in the forty-third embodiment.

In certain embodiments, the at least one compound of the present disclosure is selected from Compounds 1 to 18 shown in Table 1 below, a tautomer thereof, a deuterated derivative of the compound or the tautomer, or a pharmaceutically acceptable salt of the foregoing.

TABLE 1
1

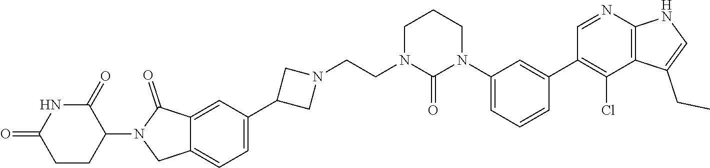 [SMILES: CCc1c[nH]c2ncc(-c3cccc(N4CCCN(CCN5CC(c6ccc7c(c6)C(=O)N(C6CCC(=O)NC6=O)C7)C5)C4=O)c3)c(Cl)c12]

2

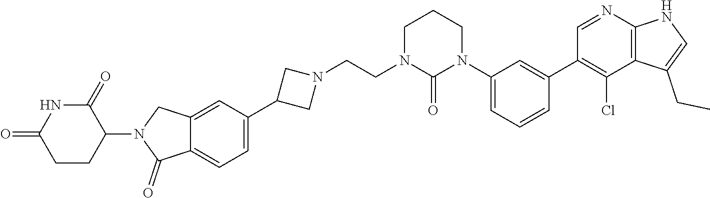 [SMILES: CCc1c[nH]c2ncc(-c3cccc(N4CCCN(CCN5CC(c6ccc7c(c6)CN(C6CCC(=O)NC6=O)C7=O)C5)C4=O)c3)c(Cl)c12]

3

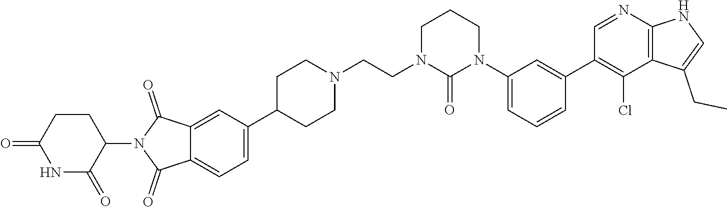 [SMILES: CCc1c[nH]c2ncc(-c3cccc(N4CCCN(CCN5CCC(c6ccc7c(c6)C(=O)N(C6CCC(=O)NC6=O)C7=O)CC5)C4=O)c3)c(Cl)c12]

4

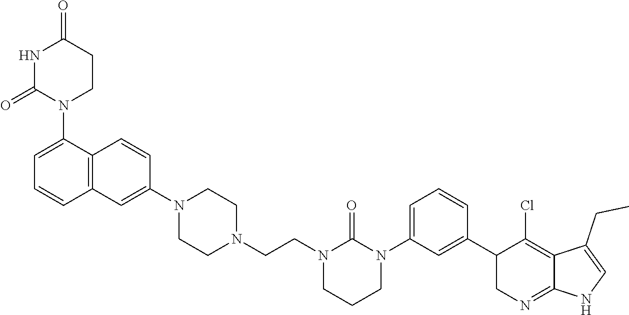 [SMILES: CCc1c[nH]c2c1=C(Cl)C(c1cccc(N3CCCN(CCN4CCN(c5ccc6c(N7CCC(=O)NC7=O)cccc6c5)CC4)C3=O)c1)CN=2]

5

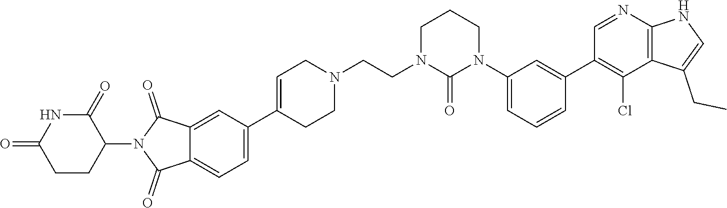 [SMILES: CCc1c[nH]c2ncc(-c3cccc(N4CCCN(CCN5CC=C(c6ccc7c(c6)C(=O)N(C6CCC(=O)NC6=O)C7=O)CC5)C4=O)c3)c(Cl)c12]

6

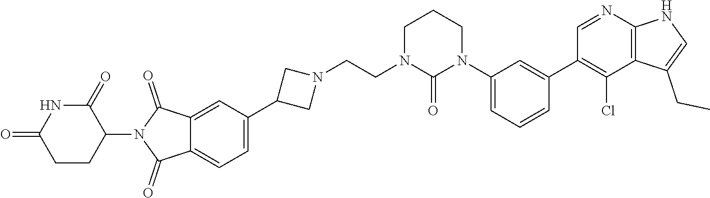 [SMILES: CCc1c[nH]c2ncc(-c3cccc(N4CCCN(CCN5CC(c6ccc7c(c6)C(=O)N(C6CCC(=O)NC6=O)C7=O)C5)C4=O)c3)c(Cl)c12]

7

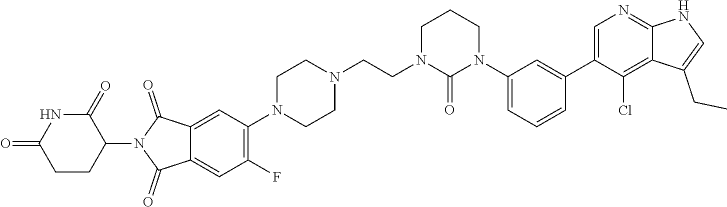 [SMILES: CCc1c[nH]c2ncc(-c3cccc(N4CCCN(CCN5CCN(c6cc7c(cc6F)C(=O)N(C6CCC(=O)NC6=O)C7=O)CC5)C4=O)c3)c(Cl)c12]

8

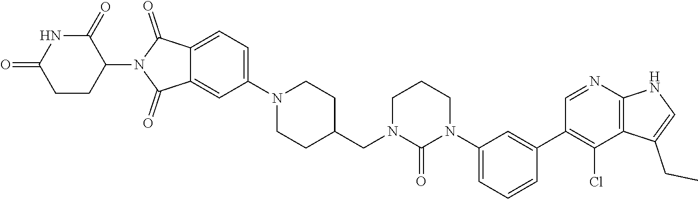 [SMILES: CCc1c[nH]c2ncc(-c3cccc(N4CCCN(CC5CCN(c6ccc7c(c6)C(=O)N(C6CCC(=O)NC6=O)C7=O)CC5)C4=O)c3)c(Cl)c12]

9

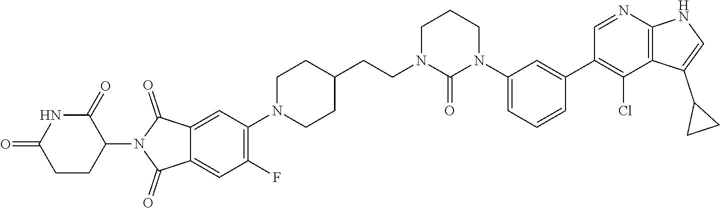 [SMILES: O=C1CCC(N2C(=O)c3cc(F)c(N4CCC(CCN5CCCN(c6cccc(-c7cnc8[nH]cc(C9CC9)c8c7Cl)c6)C5=O)CC4)cc3C2=O)C(=O)N1]

10

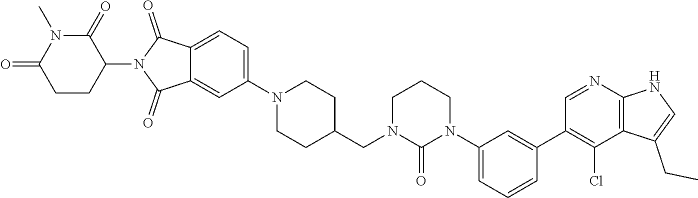 [SMILES: CCc1c[nH]c2ncc(-c3cccc(N4CCCN(CC5CCN(c6ccc7c(c6)C(=O)N(C6CCC(=O)N(C)C6=O)C7=O)CC5)C4=O)c3)c(Cl)c12]

11

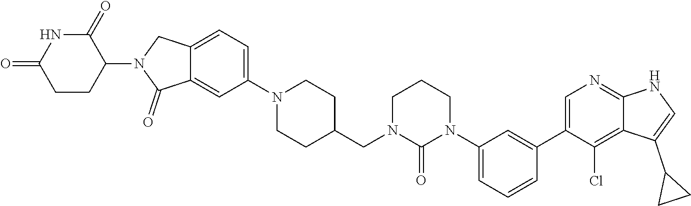 [SMILES: O=C1CCC(N2Cc3ccc(N4CCC(CN5CCCN(c6cccc(-c7cnc8[nH]cc(C9CC9)c8c7Cl)c6)C5=O)CC4)cc3C2=O)C(=O)N1]

12

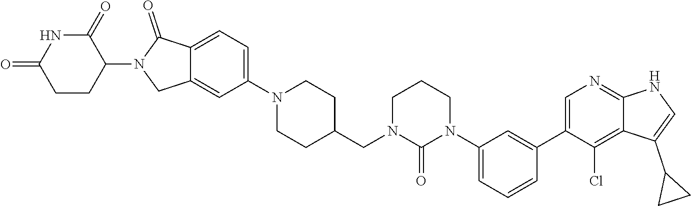 [SMILES: O=C1CCC(N2Cc3cc(N4CCC(CN5CCCN(c6cccc(-c7cnc8[nH]cc(C9CC9)c8c7Cl)c6)C5=O)CC4)ccc3C2=O)C(=O)N1]

13

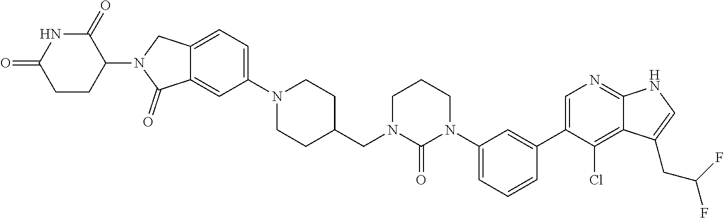 [SMILES: O=C1CCC(N2Cc3ccc(N4CCC(CN5CCCN(c6cccc(-c7cnc8[nH]cc(CC(F)F)c8c7Cl)c6)C5=O)CC4)cc3C2=O)C(=O)N1]

14

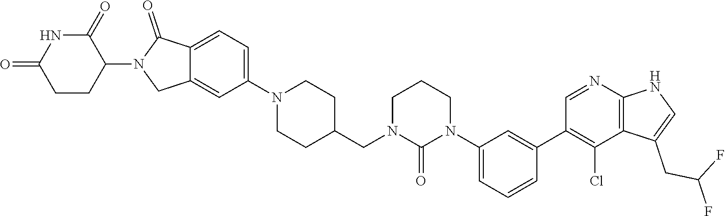 [SMILES: O=C1CCC(N2Cc3cc(N4CCC(CN5CCCN(c6cccc(-c7cnc8[nH]cc(CC(F)F)c8c7Cl)c6)C5=O)CC4)ccc3C2=O)C(=O)N1]

15

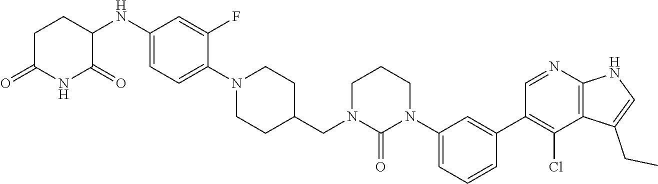 [SMILES: CCc1c[nH]c2ncc(-c3cccc(N4CCCN(CC5CCN(c6ccc(NC7CCC(=O)NC7=O)cc6F)CC5)C4=O)c3)c(Cl)c12]

16

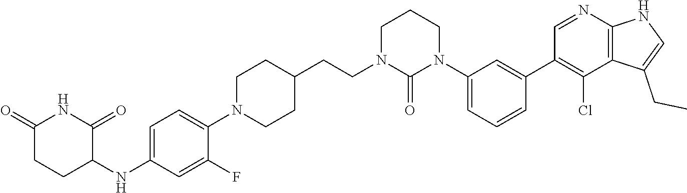 [SMILES: CCc1c[nH]c2ncc(-c3cccc(N4CCCN(CCC5CCN(c6ccc(NC7CCC(=O)NC7=O)cc6F)CC5)C4=O)c3)c(Cl)c12]

17

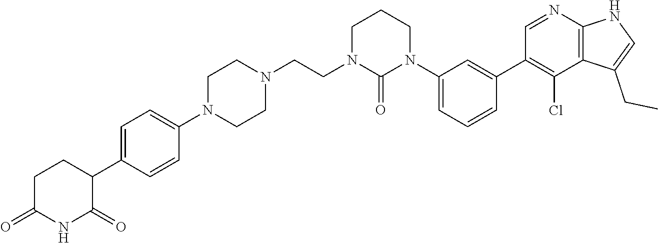 [SMILES: CCc1c[nH]c2ncc(-c3cccc(N4CCCN(CCN5CCN(c6ccc(C7CCC(=O)NC7=O)cc6)CC5)C4=O)c3)c(Cl)c12]

18

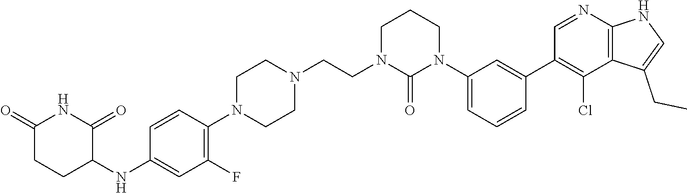 [SMILES: CCc1c[nH]c2ncc(-c3cccc(N4CCCN(CCN5CCN(c6ccc(NC7CCC(=O)NC7=O)cc6F)CC5)C4=O)c3)c(Cl)c12]

Another aspect of the present disclosure provides pharmaceutical compositions comprising at least one compound selected from a compound of Formula I, Compounds 1 to 18, a tautomer thereof, a deuterated derivative of the compound or the tautomer, or a pharmaceutically acceptable salt of the foregoing, or a pharmaceutical composition comprising any of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of the present disclosure can be employed in combination therapies; that is, the pharmaceutical compositions disclosed herein can further include an additional active pharmaceutical agent. Alternatively, a pharmaceutical composition comprising a compound selected from a compound of Formula I, Compounds 1 to 18, a tautomer thereof, a deuterated derivative of the compound or the tautomer, or a pharmaceutically acceptable salt of the foregoing, or a pharmaceutical composition comprising any of the foregoing can be administered as a separate composition concurrently with, prior to, or subsequent to, a composition comprising an additional active pharmaceutical agent.

As discussed above, the pharmaceutical compositions disclosed herein comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams &Wilkins, Philadelphia, and Encyclopedia of Pharmaceutical Technology, eds. J. Swarbrick and J. C. Boylan, 1988 to 1999, Marcel Dekker, New York disclose various carriers used in formulating pharmaceutical compositions and known techniques for the preparation thereof. Except insofar as any conventional carrier is incompatible with the compounds of the present disclosure, such as by producing any undesirable biological effect or otherwise interacting in a deleterious manner with any other component (s) of the pharmaceutical composition, its use is contemplated to be within the scope of the present disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### III. Methods of Treatment and Uses

In another aspect of the present disclosure, a compound, tautomer, deuterated derivative, or pharmaceutically acceptable salt as disclosed herein, including a compound of Formula I, Compounds 1 to 18, a tautomer thereof, a deuterated derivative of the compound or the tautomer, or a pharmaceutically acceptable salt of the foregoing, or the pharmaceutical composition thereof, is for use in treating a disease, a disorder, or a condition mediated by the degradation of a protein kinase. In another aspect, disclosed herein is use of the compound, tautomer, deuterated derivative, and/or the pharmaceutically acceptable salt thereof as disclosed herein, including a compound of Formula I, Compounds 1 to 18, a tautomer thereof, a deuterated derivative of the compound or the tautomer, and/or a pharmaceutically acceptable salt of the foregoing, or the pharmaceutical composition thereof, for the manufacture of a medicament for treating a disease, a disorder, or a condition mediated by the degradation of a protein kinase. In yet another aspect, disclosed herein is a method of treating a disease, a disorder, or a condition mediated by the degradation of protein kinase in a subject, comprising administering a therapeutically effective amount of a compound, tautomer, deuterated derivative, and/or pharmaceutically acceptable salt as disclosed herein, including a compound of Formula I, Compounds 1 to 18, a tautomer thereof, a deuterated derivative of the compound or the tautomer, and/or a pharmaceutically acceptable salt of the foregoing, or the pharmaceutical composition thereof.

In some embodiments, the protein kinase is chosen from hematopoietic progenitor kinase 1 (HPK1), mitogen-activated protein kinases 1/2 (MEK 1/2), Fms-like tyrosine kinase 3 receptor (FLT3), and Aurora A.

In some embodiments, the disease, the disorder, or the condition is chosen from protein kinase-related diseases. In some embodiments, the disease, the disorder, or the condition is chosen from MEK 1/2-related diseases. In some embodiments, the disease, the disorder, or the condition is chosen from FLT3-related diseases. In some embodiments, the disease, the disorder, or the condition is chosen from Aurora A-related diseases.

In some embodiments, the disease, the disorder, or the condition is cancer. In some embodiments, the cancer is a solid tumor. In some embodiments, the solid tumor is chosen from brain cancer, breast cancer, respiratory tract and/or lung cancer, a reproductive organ cancer, bone cancer, digestive tract cancer, urinary tract cancer, eye cancer, liver cancer, skin cancer, head and neck cancer, anal cancer, nervous system cancer, thyroid cancer, and parathyroid cancer. In some embodiments, the cancer is a hematologic cancer. In some embodiments, the hematologic cancer is chosen from acute myeloid leukemia (AML), acute lymphoblastic leukemia (ALL), multiple myeloma (MM), diffuse large B-cell lymphoma (DLBCL), non-Hodgkin's lymphoma (NHL), Hodgkin's lymphoma mesothelioma (HL), T-cell lymphoma (TCL), Burkitt lymphoma (BL), chronic lymphocytic leukemia/small lymphocytic lymphoma (CLL/SLL), mantle cell lymphoma (MCL), marginal zone lymphoma (MZL), and myelodysplastic syndromes (MDS).

In some embodiments, the cancer is chosen from cancers of epidermoid oral such as buccal cavity, lip, tongue, mouth, pharynx; cardiac cancers such as sarcoma (angiosarcoma, fibrosarcoma, rhabdomyosarcoma, liposarcoma), myxoma, rhabdomyoma, fibroma, lipoma, and teratoma; lung cancers such as bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatosis hamartoma, mesothelioma; gastrointestinal cancers such as esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Kaposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers such as hepatoma (hepatocellular carcinoma), cholangiocarcinoma, hepatoblastoma, angiosarcoma, hepatocellular adenoma, hemangioma, biliary passages; bone cancers such as osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; cancers of the nervous system, including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosathecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers such as blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma) hairy cell; lymphoid disorders; skin cancers including malignant melanoma, basal cell carcinoma, squamous cell carcinoma, Kaposi's sarcoma, keratoacanthoma, moles dysplastic nevi, lipoma, angioma, dermatofibroma, keloids, psoriasis; cancers of the thyroid gland such as papillary thyroid carcinoma, follicular thyroid carcinoma; medullary' thyroid carcinoma, undifferentiated thyroid cancer, multiple endocrine neoplasia type 2A, multiple endocrine neoplasia type 2B, familial medullary thyroid cancer, pheochromocytoma, paraganglioma; and cancers of the adrenal glands like neuroblastoma.

In another aspect of the present disclosure, a compound, tautomer, deuterated derivative, and/or pharmaceutically acceptable salt as disclosed herein, including a compound of Formula I, Compounds 1 to 18, a tautomer thereof, a deuterated derivative of the compound or the tautomer, and/or a pharmaceutically acceptable salt of the foregoing, or the pharmaceutical composition thereof, is for use in decreasing protein kinase activity. In another aspect, disclosed herein is use of a compound, tautomer, deuterated derivative, and/or pharmaceutically acceptable salt as disclosed herein, including a compound of Formula I, Compounds 1 to 18, a tautomer thereof, a deuterated derivative of the compound or the tautomer, and/or a pharmaceutically acceptable salt of the foregoing, or the pharmaceutical composition thereof, for the manufacture of a medicament for decreasing protein kinase activity. In yet another aspect, disclosed herein is a method of decreasing protein kinase activity, comprising administering a therapeutically effective amount of a compound, tautomer, deuterated derivative, and/or pharmaceutically acceptable salt as disclosed herein to a subject, including a compound of Formula I, Compounds 1 to 18, a tautomer thereof, a deuterated derivative of the compound or the tautomer, and/or a pharmaceutically acceptable salt of the foregoing, or the pharmaceutical composition thereof. In yet another aspect, disclosed herein is a method of decreasing protein kinase activity, comprising contacting said protein kinase with a compound, tautomer, deuterated derivative, and/or pharmaceutically acceptable salt as disclosed herein to a subject, including a compound of Formula I, Compounds 1 to 18, a tautomer thereof, a deuterated derivative of the compound or the tautomer, and/or a pharmaceutically acceptable salt of the foregoing, or the pharmaceutical composition thereof.

A compound of Formula I, Compounds 1 to 18, a tautomer thereof, a deuterated derivative of the compound or the tautomer, and/or a pharmaceutically acceptable salt of the foregoing, or the pharmaceutical composition thereof may be administered once daily, twice daily, or three times daily, for example, for the treatment of a disease, a disorder, or a condition mediated by the degradation of protein kinase.

In some embodiments, 2 mg to 1500 mg or 5 mg to 1000 mg of a compound of Formula I, Compounds 1 to 18, a tautomer thereof, a deuterated derivative of the compound or the tautomer, and/or a pharmaceutically acceptable salt of the foregoing, or the pharmaceutical composition thereof are administered once daily, twice daily, or three times daily.

A compound of Formula I, Compounds 1 to 18, a tautomer thereof, a deuterated derivative of the compound or the tautomer, and/or a pharmaceutically acceptable salt of the foregoing, or the pharmaceutical composition thereof may be administered, for example, by oral, parenteral, sublingual, topical, rectal, nasal, buccal, vaginal, transdermal, patch, pump administration or via an implanted reservoir, and the pharmaceutical compositions would be formulated accordingly. Parenteral administration includes, for example, intravenous, intraperitoneal, subcutaneous, intramuscular, transepithelial, nasal, intrapulmonary, intrathecal, rectal and topical modes of administration. Parenteral administration can, for example, be by continuous infusion over a selected period of time. Other forms of administration contemplated in the present disclosure are as described in International Patent Application Nos. WO 2013/075083, WO 2013/075084, WO 2013/078320, WO 2013/120104, WO 2014/124418, WO 2014/151142, and WO 2015/023915.

Useful dosages or a therapeutically effective amount of a compound or pharmaceutically acceptable salt thereof as disclosed herein can be determined by comparing their in vitro activity and in vivo activity in animal models. Methods for the extrapolation of effective dosages in mice and other animals to humans are known in the art. See, e.g., U.S. Pat. No. 4,938,949.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, tautomers, pharmaceutically acceptable salts, and deuterated derivatives disclosed herein are based upon the free base form of the reference compound. For example, “1000 mg of at least one compound chosen from compounds of Formula I and pharmaceutically acceptable salts thereof” includes 1000 mg of compound of Formula I and a concentration of a pharmaceutically acceptable salt of compounds of Formula I equivalent to 1000 mg of compounds of Formula I.

In another aspect of the present disclosure, the compounds and the compositions disclosed herein can be administered in therapeutically effective amounts in a combinational therapy with one or more therapeutic agents (pharmaceutical combinations) or modalities, e.g., anti-proliferative, anti-cancer, immunomodulatory or anti-inflammatory agent, and/or non-drug therapies, etc. For example, synergistic effects can occur with anti-proliferative, anti-cancer, immunomodulatory or anti-inflammatory substances. Where the compounds disclosed herein are administered in conjunction with other therapies, dosages of the co-administered compounds will of course vary depending on the type of co-drug employed, on the specific drug employed, on the condition being treated and so forth. Combination therapy includes the administration of the subject compounds in further combination with one or more other biologically active ingredients (such as a second kinase inhibitor, a second and different antineoplastic agent, and non-drug therapies (such as surgery or radiation treatment). For instance, the compounds disclosed herein can be used in combination with other pharmaceutically active compounds, preferably compounds that are able to enhance the effect of the compounds disclosed herein. The compounds disclosed herein can be administered simultaneously (as a single preparation or separate preparation) or sequentially to the other drug therapy or treatment modality. In general, a combination therapy envisions administration of two or more drugs during a single cycle or course of therapy. In another aspect of the disclosure, the compounds may be administered in combination with one or more separate pharmaceutical agents, e.g., a chemotherapeutic agent, an immunotherapeutic agent, or an adjunctive therapeutic agent. In an embodiment, the separate pharmaceutical agent is selected from an anti-PD1 antibody (e.g. pembrolizumab), an HDAC inhibitor r (e.g. panobinostat, romidepsin, vorinostat, or citarinostat), a BCL-2 inhibitor (e.g. venetoclax), a BTK inhibitor (e.g. ibrutinib or acalabrutinib), an mTOR inhibitor (e.g. everolimus), a PI3K inhibitor r (e.g. idelalisib), a PKCβ inhibitor (e.g. enzastaurin), a SYK inhibitor (e.g. fostamatinib), a JAK2 inhibitor (e.g. fedratinib, pacritinib, ruxolitinib, baricitinib, gandotinib, lestaurtinib, or momelotinib), an Aurora kinase inhibitor (e.g. alisertib), an EZF12 inhibitor (e.g. tazemetostat, GSK126, CPI-1205, 3-deazaneplanocin A, EPZ005687, Ell, UNC1999, or sinefungin), a BET inhibitor (e.g. birabresib), a hypomethylating agent (e.g. 5-azacytidine or decitabine), a DOT1L inhibitor (e.g. pinometostat), a FIAT inhibitor (e.g. C646), a WDR5 inhibitor (e.g. OICR-9429), a DNMTl inhibitor (e.g. GSK3484862), an LSD-1 inhibitor (e.g. Compound C or seclidemstat), a G9A inhibitor (e.g. UNC0631), a PRMT5 inhibitor (e.g. GSK3326595), a BRD inhibitor (e.g. LP99), a SUV420FU/F12 inhibitor (e.g. A-196), a CARM1 inhibitor (e.g. EZM2302), a PLK1 inhibitor (e.g. B12536), an NEK2 inhibitor (e.g. JF1295), an MEK inhibitor (e.g. trametinib, binimetinib, cobimetinib, selumetinib), a PF1F19 inhibitor, a PIM inhibitor (e.g. LGF1-447), an IGF-IR inhibitor (e.g. linsitinib), an XPOl inhibitor (e.g. selinexor), a BIRC5 inhibitor (e.g. YMl 55), a PARP inhibitor (e.g. Olaparib), an EGFR inhibitor (e.g. Osimertinib), a HER2/NEU inhibitor (i.e. tucatinib), an SRC inhibitor (i.e. dasatinib), an AKT inhibitor (i.e. Ipatasertib), a KRAS (G12C) inhibitor (i.e. Sotorasib), a KRAS (G12D) inhibitor (i.e. MRTX1133), platinum, or a chemotherapy (e.g, bendamustine, bleomycin, doxorubicin, etoposide, methotrexate, cytarabine, vincristine, ifosfamide, melphalan, oxaliplatin, cisplatin, taxanes or dexamethasone).

### Non-Limiting Exemplary Embodiments

1. A compound of Formula (I):

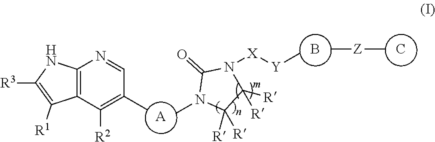

a tautomer thereof, a deuterated derivative of the compound or the tautomer, or a pharmaceutically acceptable salt of the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R;
(ii) R² and R³ are independently chosen from hydrogen, halogen groups, OR^{x}, SR^{x}, NHR^{x}, N(R^{x})₂, CHR^{x}, and C(R^{x})₂;
(iii) each R is independently chosen from hydrogen, halogen groups, linear, branched, and cyclic alkyl groups;
(iv) m and n are independently chosen from 0, 1, and 2;
(v) X is absent or is chosen from linear, branched, cyclic alkylene groups, linear, branched, and cyclic heteroalkylene groups;
(vi) Y and Z are independently absent or chosen from —O—, —C(O)—, —C(O)R^{x}—, —C(S)—, —C(S)R^{x}—, —[C(R^{x}R^{y})]ₚ—, —S(O)₂—, —S(O)₂R^{x}—, NR^{x}—, and —NR^{x}C(O)—, wherein p is chosen from 1, 2, 3, 4, 5, and 6; wherein if X is absent, then Y is not —O—, —S(O)₂—, —S(O)₂R^{x}—, NR^{x}—, or —NR^{x}C(O)—;
(vii) R^{x}, R^{y}, and R^{w} are each independently chosen from hydrogen, linear, branched, and cyclic alkyl groups, carbocyclic groups, heterocyclic groups, aryl groups, and heteroaryl groups;
(viii) ring A is chosen from optionally substituted aryl groups and heteroaryls groups,
(ix) ring B is absent or is chosen from cycloalkyl groups and heterocycloalkyls;
(x) ring C is chosen from

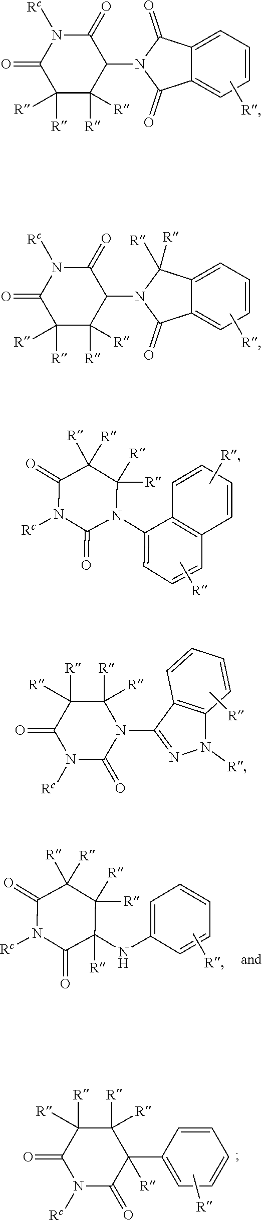

wherein R^{c} is chosen from hydrogen, linear, branched, and cyclic alkyl groups, and pro-drug groups; R″ is chosen from hydrogen, halogen groups, OR^{x}, linear, branched, and cyclic alkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O) C₁-C₆ linear, branched, and cyclic alkyl groups,
—C(O) OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O) C₁-C₆ linear, branched, and cyclic alkyl groups,
—C(O) NHC₁-C₆ linear, branched, and cyclic alkyl groups,
—NHaryl groups,
—N(aryl groups)₂,
—NHC(O) aryl groups,
—C(O) NHaryl groups,
—NHheteroaryl groups,
—N(heteroaryl groups)₂,
—NHC(O) heteroaryl groups,
—C(O) NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.
2. The compound, tautomer, deuterated derivative, or pharmaceutically acceptable salt of embodiment 1, wherein R¹ is chosen from linear, branched, and cyclic alkyl groups; R² is a halogen group; and R³ is chosen from hydrogen, linear, branched, and cyclic alkyl groups.
3. The compound, tautomer, deuterated derivative, or pharmaceutically acceptable salt of embodiment 1 or 2, wherein R¹ is chosen from C₁-C₆ linear, branched, and cyclic alkyl groups.
4. The compound, tautomer, deuterated derivative, or pharmaceutically acceptable salt of embodiment 3, wherein R¹ is chosen from methyl, ethyl, cyclopropyl, and cyclobutyl.
5. The compound, tautomer, deuterated derivative, or pharmaceutically acceptable salt of any of embodiments 1-4, wherein R² is a halogen group.
6. The compound, tautomer, deuterated derivative, or pharmaceutically acceptable salt of embodiment 5, wherein R² is chloro.
7. The compound, tautomer, deuterated derivative, or pharmaceutically acceptable salt of any of embodiments 1-5, wherein R² is hydrogen.
8. The compound, tautomer, deuterated derivative, or pharmaceutically acceptable salt of any of embodiments 1-7, wherein R³ is a halogen group.
9. The compound, tautomer, deuterated derivative, or pharmaceutically acceptable salt of embodiment 8, wherein R³ is chloro.
10. The compound, tautomer, deuterated derivative, or pharmaceutically acceptable salt of any of embodiments 1-7, wherein R³ is hydrogen.
11. The compound, tautomer, deuterated derivative, or pharmaceutically acceptable salt of any of embodiments 1-10, wherein m is 1 and n is 1.
12. The compound, tautomer, deuterated derivative, or pharmaceutically acceptable salt of embodiment 11, wherein each R′ is hydrogen.
13. The compound, tautomer, deuterated derivative, or pharmaceutically acceptable salt of any of embodiments 1-10, wherein m is 2 and n is 1.
14. The compound, tautomer, deuterated derivative, or pharmaceutically acceptable salt of embodiment 11, wherein each R′ is hydrogen.
15. The compound, tautomer, deuterated derivative, or pharmaceutically acceptable salt of any of embodiments 1-14, wherein X is absent.
16. The compound, tautomer, deuterated derivative, or pharmaceutically acceptable salt of any of embodiments 1-14, wherein X is a linear alkylene group.
17. The compound, tautomer, deuterated derivative, or pharmaceutically acceptable salt of embodiment 16, wherein X is a methylene group.
18. The compound, tautomer, deuterated derivative, or pharmaceutically acceptable salt of embodiment 16, wherein X is an ethylene group.
19. The compound, tautomer, deuterated derivative, or pharmaceutically acceptable salt of any of embodiments 1-18, wherein Y is absent.
20. The compound, tautomer, deuterated derivative, or pharmaceutically acceptable salt of any of embodiments 1-19, wherein ring B is chosen from optionally substituted heterocycloalkyls.
21. The compound, tautomer, deuterated derivative, or pharmaceutically acceptable salt of embodiment 20, wherein ring B is chosen from

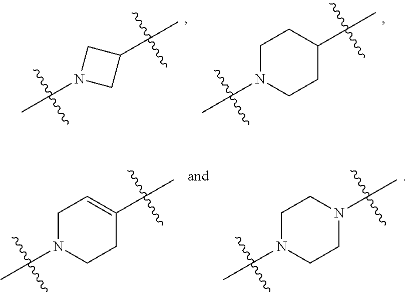 [SMILES: *C1CN(*)C1 ; *C1CCN(*)CC1 ; *C1=CCN(*)CC1 ; *N1CCN(*)CC1]

22. The compound, tautomer, deuterated derivative, or pharmaceutically acceptable salt of any of embodiments 1-21, wherein Z is absent.
23. The compound, tautomer, deuterated derivative, or pharmaceutically acceptable salt of any of embodiments 1-22, wherein ring C is

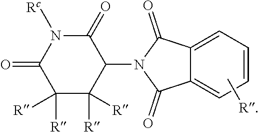

24. The compound, tautomer, deuterated derivative, or pharmaceutically acceptable salt of embodiment 23, wherein R^{c} is chosen from hydrogen, linear, branched, and cyclic alkyl groups, and pro-drug groups.
25. The compound, tautomer, deuterated derivative, or pharmaceutically acceptable salt of any of embodiments 1-22, wherein ring C is

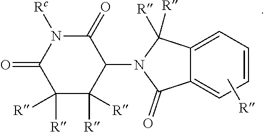

26. The compound, tautomer, deuterated derivative, or pharmaceutically acceptable salt of embodiment 25, wherein R^{c} is chosen from hydrogen, linear, branched, and cyclic alkyl groups, and pro-drug groups.
27. The compound, tautomer, deuterated derivative, or pharmaceutically acceptable salt of any of embodiments 1-22, wherein ring C is

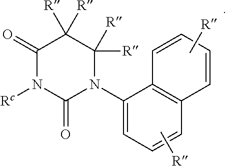

28. The compound, tautomer, deuterated derivative, or pharmaceutically acceptable salt of embodiment 27, wherein R^{c} is chosen from hydrogen, linear, branched, and cyclic alkyl groups, and pro-drug groups.
29. The compound, tautomer, deuterated derivative, or pharmaceutically acceptable salt of any of embodiments 1-22, wherein ring C is

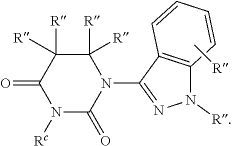

30. The compound, tautomer, deuterated derivative, or pharmaceutically acceptable salt of embodiment 29, wherein R^{c} is chosen from hydrogen, linear, branched, and cyclic alkyl groups, and pro-drug groups.
31. The compound, tautomer, deuterated derivative, or pharmaceutically acceptable salt of any of embodiments 1-22, wherein ring C is

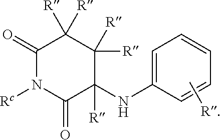

32. The compound, tautomer, deuterated derivative, or pharmaceutically acceptable salt of embodiment 31, wherein R^{c} is chosen from hydrogen, linear, branched, and cyclic alkyl groups, and pro-drug groups.
33. The compound, tautomer, deuterated derivative, or pharmaceutically acceptable salt of any of embodiments 1-22, wherein ring C is

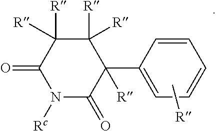

34. The compound, tautomer, deuterated derivative, or pharmaceutically acceptable salt of embodiment 33, wherein R^{c} is chosen from hydrogen, linear, branched, and cyclic alkyl groups, and pro-drug groups.
35. A compound chosen from

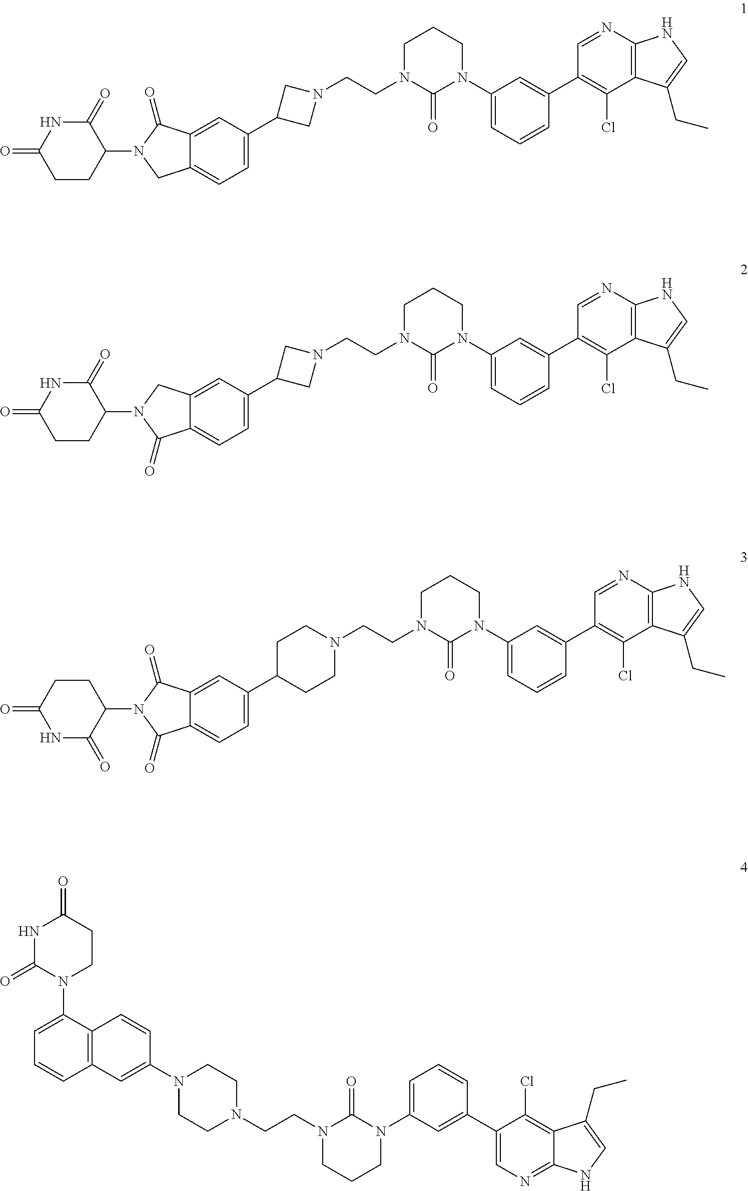 [SMILES: CCc1c[nH]c2ncc(-c3cccc(N4CCCN(CCN5CC(c6ccc7c(c6)C(=O)N(C6CCC(=O)NC6=O)C7)C5)C4=O)c3)c(Cl)c12 ; CCc1c[nH]c2ncc(-c3cccc(N4CCCN(CCN5CC(c6ccc7c(c6)CN(C6CCC(=O)NC6=O)C7=O)C5)C4=O)c3)c(Cl)c12 ; CCc1c[nH]c2ncc(-c3cccc(N4CCCN(CCN5CCC(c6ccc7c(c6)C(=O)N(C6CCC(=O)NC6=O)C7=O)CC5)C4=O)c3)c(Cl)c12 ; CCc1c[nH]c2ncc(-c3cccc(N4CCCN(CCN5CCN(c6ccc7c(N8CCC(=O)NC8=O)cccc7c6)CC5)C4=O)c3)c(Cl)c12]

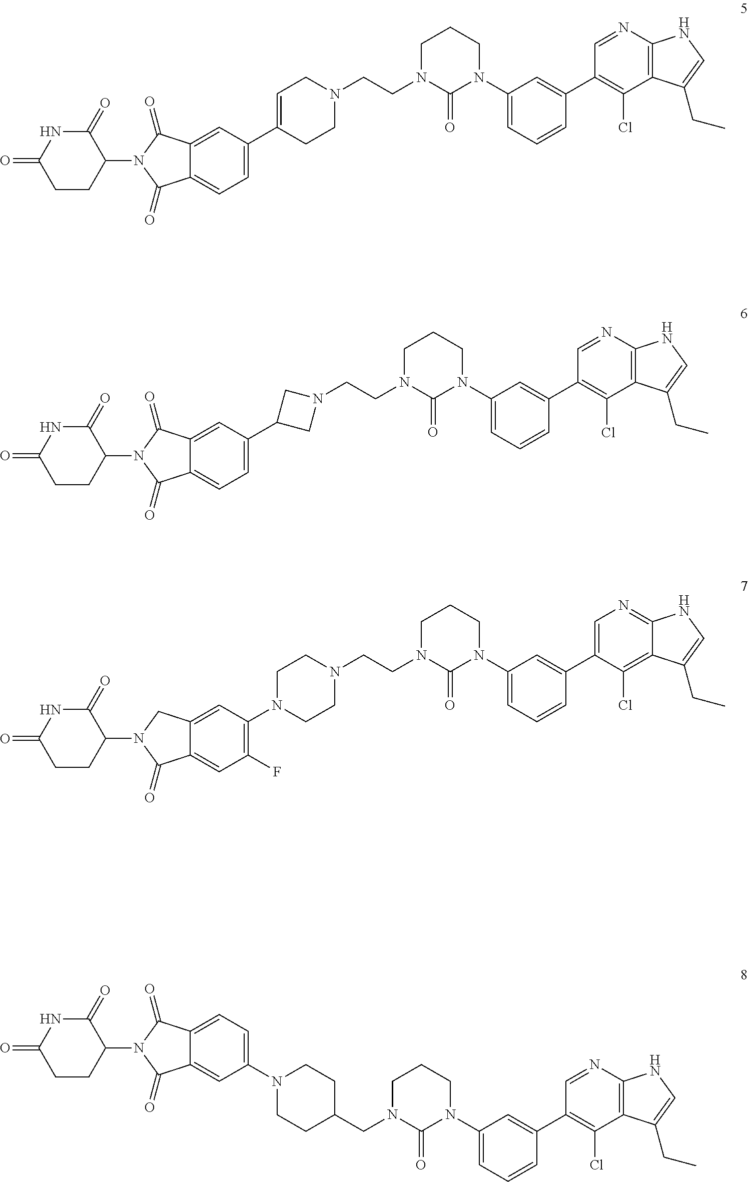 [SMILES: CCc1c[nH]c2ncc(-c3cccc(N4CCCN(CCN5CC=C(c6ccc7c(c6)C(=O)N(C6CCC(=O)NC6=O)C7=O)CC5)C4=O)c3)c(Cl)c12 ; CCc1c[nH]c2ncc(-c3cccc(N4CCCN(CCN5CC(c6ccc7c(c6)C(=O)N(C6CCC(=O)NC6=O)C7=O)C5)C4=O)c3)c(Cl)c12 ; CCc1c[nH]c2ncc(-c3cccc(N4CCCN(CCN5CCN(c6cc7c(cc6F)C(=O)N(C6CCC(=O)NC6=O)C7)CC5)C4=O)c3)c(Cl)c12 ; CCc1c[nH]c2ncc(-c3cccc(N4CCCN(CC5CCN(c6ccc7c(c6)C(=O)N(C6CCC(=O)NC6=O)C7=O)CC5)C4=O)c3)c(Cl)c12]

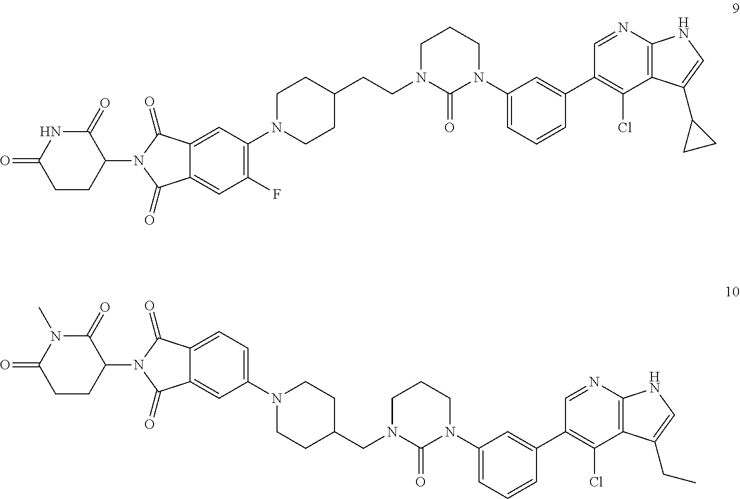 [SMILES: O=C1CCC(N2C(=O)c3cc(F)c(N4CCC(CCN5CCCN(c6cccc(-c7cnc8[nH]cc(C9CC9)c8c7Cl)c6)C5=O)CC4)cc3C2=O)C(=O)N1 ; CCc1c[nH]c2ncc(-c3cccc(N4CCCN(CC5CCN(c6ccc7c(c6)C(=O)N(C6CCC(=O)N(C)C6=O)C7=O)CC5)C4=O)c3)c(Cl)c12]

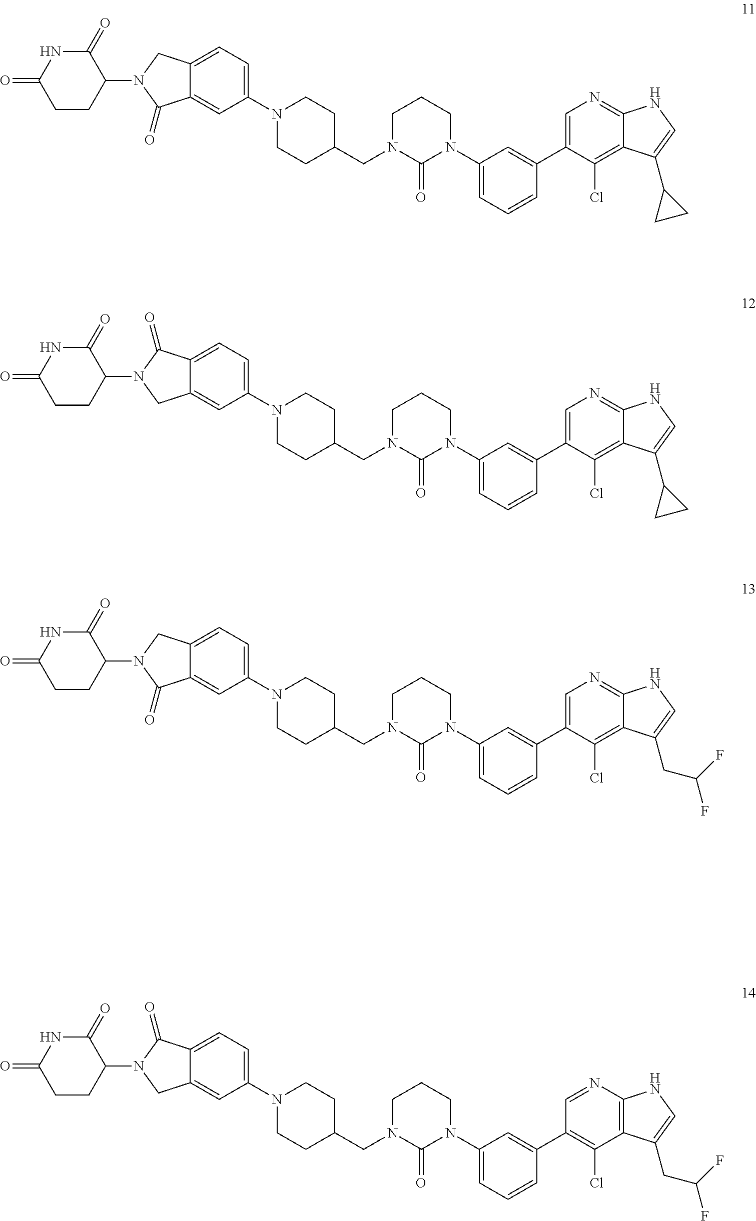 [SMILES: O=C1CCC(N2Cc3ccc(N4CCC(CN5CCCN(c6cccc(-c7cnc8[nH]cc(C9CC9)c8c7Cl)c6)C5=O)CC4)cc3C2=O)C(=O)N1 ; O=C1CCC(N2Cc3cc(N4CCC(CN5CCCN(c6cccc(-c7cnc8[nH]cc(C9CC9)c8c7Cl)c6)C5=O)CC4)ccc3C2=O)C(=O)N1 ; O=C1CCC(N2Cc3ccc(N4CCC(CN5CCCN(c6cccc(-c7cnc8[nH]cc(CC(F)F)c8c7Cl)c6)C5=O)CC4)cc3C2=O)C(=O)N1 ; O=C1CCC(N2Cc3cc(N4CCC(CN5CCCN(c6cccc(-c7cnc8[nH]cc(CC(F)F)c8c7Cl)c6)C5=O)CC4)ccc3C2=O)C(=O)N1]

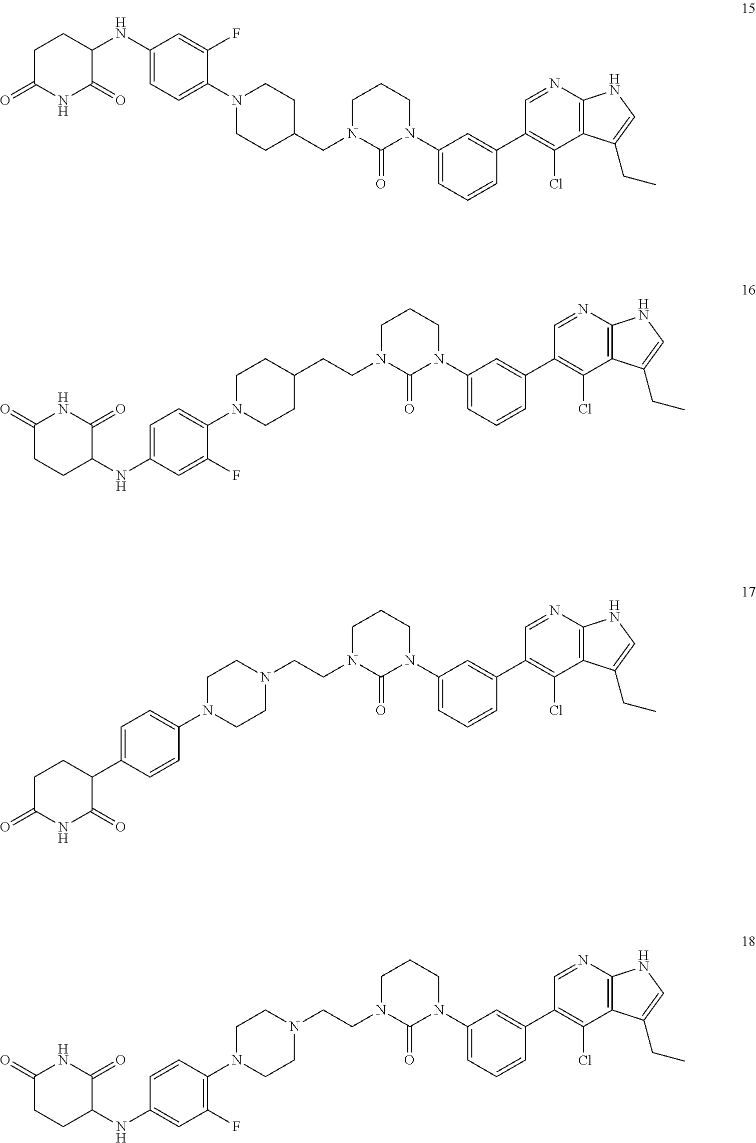 [SMILES: CCc1c[nH]c2ncc(-c3cccc(N4CCCN(CC5CCN(c6ccc(NC7CCC(=O)NC7=O)cc6F)CC5)C4=O)c3)c(Cl)c12 ; CCc1c[nH]c2ncc(-c3cccc(N4CCCN(CCC5CCN(c6ccc(NC7CCC(=O)NC7=O)cc6F)CC5)C4=O)c3)c(Cl)c12 ; CCc1c[nH]c2ncc(-c3cccc(N4CCCN(CCN5CCN(c6ccc(C7CCC(=O)NC7=O)cc6)CC5)C4=O)c3)c(Cl)c12 ; CCc1c[nH]c2ncc(-c3cccc(N4CCCN(CCN5CCN(c6ccc(NC7CCC(=O)NC7=O)cc6F)CC5)C4=O)c3)c(Cl)c12]

a tautomer thereof, a deuterated derivative of the compound or the tautomer, or a pharmaceutically acceptable salt of the foregoing.
36. A pharmaceutical composition comprising a compound, tautomer, deuterated derivative, and/or pharmaceutically acceptable salt according to any one of embodiments 1-35 and at least one pharmaceutically acceptable carrier.
37. A method for treating or alleviating a disease, a disorder or a condition mediated by the degradation of a protein kinase, comprising administering to a subject in need thereof a therapeutically effective amount of a compound, tautomer, deuterated derivative, and/or pharmaceutically acceptable salt according to any one of the embodiments 1-35 or the pharmaceutical composition according to embodiment 36.
38. The method of embodiment 37, wherein the protein kinase is chosen from hematopoietic progenitor kinase 1 (HPK1), mitogen-activated protein kinases 1/2 (MEK 1/2), Fms-like tyrosine kinase 3 receptor (FLT3), and Aurora A.
39. A method for decreasing a protein kinase activity in a disease, a disorder or a condition, comprising administering to a subject in need thereof a therapeutically effective amount of a compound, tautomer, deuterated derivative, and/or pharmaceutically acceptable salt according to any one of the embodiments 1-35 or the pharmaceutical composition according to embodiment 36.
40. The method of embodiment 39, wherein the disease, the disorder, or the condition is chosen from a protein kinase-related disease.
41. The method of embodiment 40, wherein the protein kinase-related disease is cancer.
42. The method of embodiment 41, wherein the cancer is a solid tumor.
43. The method of embodiment 42, wherein the solid tumor is chosen from brain cancer, breast cancer, respiratory tract and/or lung cancer, a reproductive organ cancer, bone cancer, digestive tract cancer, urinary tract cancer, eye cancer, liver cancer, skin cancer, head and neck cancer, anal cancer, nervous system cancer, thyroid cancer, and parathyroid cancer.
44. The method of embodiment 41, wherein the cancer is a hematologic cancer.
45. The method of embodiment 44, wherein the hematologic cancer is chosen from acute myeloid leukemia (AML), acute lymphoblastic leukemia (ALL), multiple myeloma (MM), diffuse large B-cell lymphoma (DLBCL), non-Hodgkin's lymphoma (NHL), Hodgkin's lymphoma mesothelioma (HL), T-cell lymphoma (TCL), Burkitt lymphoma (BL), chronic lymphocytic leukemia/small lymphocytic lymphoma (CLL/SLL), mantle cell lymphoma (MCL), marginal zone lymphoma (MZL), and myelodysplastic syndromes (MDS).
46. The method of embodiment 41, wherein the cancer is chosen from epidermoid oral such as buccal cavity, lip, tongue, mouth, pharynx; cardiac cancers such as sarcoma (angiosarcoma, fibrosarcoma, rhabdomyosarcoma, liposarcoma), myxoma, rhabdomyoma, fibroma, lipoma, and teratoma; lung cancers such as bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatosis hamartoma, mesothelioma; gastrointestinal cancers such as esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Kaposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers such as hepatoma (hepatocellular carcinoma), cholangiocarcinoma, hepatoblastoma, angiosarcoma, hepatocellular adenoma, hemangioma, biliary passages; bone cancers such as osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; cancers of the nervous system, including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosathecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers such as blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma) hairy cell; lymphoid disorders; skin cancers including malignant melanoma, basal cell carcinoma, squamous cell carcinoma, Kaposi's sarcoma, keratoacanthoma, moles dysplastic nevi, lipoma, angioma, dermatofibroma, keloids, psoriasis; cancers of the thyroid gland such as papillary thyroid carcinoma, follicular thyroid carcinoma; medullary' thyroid carcinoma, undifferentiated thyroid cancer, multiple endocrine neoplasia type 2A, multiple endocrine neoplasia type 2B, familial medullary thyroid cancer, pheochromocytoma, paraganglioma; and cancers of the adrenal glands like neuroblastoma.
47. The method of embodiment 39, further comprising the administration to the subject an existing standard treatment or an FDA-approved therapy.
48. The method of embodiment 39, further comprising the administration to the subject one or more separate pharmaceutical agents.
49. The method of embodiment 48, wherein the separate pharmaceutical agent is chosen from a chemotherapeutic agent, an immunotherapeutic agent, and an adjunctive therapeutic agent.

### EXAMPLES

### Synthesis of Compounds

To fully understand the present disclosure, the following examples are disclosed. It should be understood that these examples are for illustrative purposes only and are not to be construed as limiting the present disclosure in any manner.

All the specific and generic compounds, and the intermediates disclosed for making those compounds, are considered to be part of the present disclosure.

The compounds of the present disclosure may be made according to standard chemical practices or as disclosed herein. Throughout the following synthetic schemes and in the descriptions for preparing compounds of Formula I, Compounds 1 to 18, pharmaceutically acceptable salts of any of those compounds, solvates of any of the foregoing, and deuterated derivatives of any of the foregoing, the following abbreviations are used:

### Abbreviations

A=angstrom
Ac=acetyl
Ac₂O=acetic anhydride
Boc₂O=di-tert-butyl dicarbonate
DCM=dichloromethane
DIEA=N, N-Diisopropylethylamine or N-ethyl-N-isopropyl-propan-2-amine
DMAP=dimethylamino pyridine
DMA=dimethyl acetamide
DME=dimethoxyethane
DMF=dimethylformamide
DMSO=dimethyl sulfoxide
EtOAc/EA=Ethyl Acetate
EtOH=ethanol
HOAc=acetic acid
KOAc=potassium acetate
LiHMDS=lithium bis(trimethylsilyl) amide
MeMgBr=methylmagnesium bromide
MeOH=methanol
NaOAc=sodium acetate
NBS=N-bromosuccinimide
Pd(dppf)₂Cl₂=[1,1′-Bis (diphenylphosphino) ferrocene] dichloropalladium (II)
PTSA=p-Toluenesulfonic acid monohydrate
rt=room (ambient) temperature
T3P=2, 4, 6-Tripropyl-1, 3, 5, 2, 4, 6-trioxatriphosphorinane-2, 4, 6-trioxide
TEA=triethylamine
TFA=trifluoroacetic acid
THF=tetrahydrofuran
TsCl=p-toluene sulfonyl chloride
UV=ultra-violet
X-Phos=2-dicyclohexylphosphino-2′, 4′, 6′-triisopropylbiphenyl

### Synthesis of Intermediates Compounds

### Intermediate A1: 5-bromo-4-chloro-3-ethyl-1H-pyrrolo [2, 3-b] pyridine

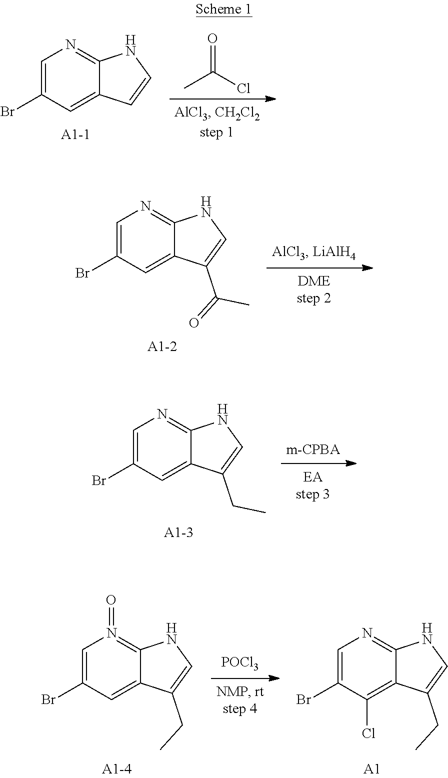

### Step 1. Preparation of 1-(5-bromo-1H-pyrrolo [2, 3-b] pyridin-3-yl) ethan-1-one

To a solution of 5-bromo-1H-pyrrolo [2, 3-b] pyridine (50 g, 0.25 mol) in DCM (550 mL) was added AlCl₃ (101.27 g, 0.76 mol) and acetyl chloride (21.92 g, 0.28 mol) at 0° C. under N₂. The reaction mixture was stirred at rt under N₂ for 7 hrs. MeOH (300 mL) was added to the reaction mixture and the solvent was removed under reduced pressure. The reaction solution was adjusted to pH 6-7 with 3 N aqueous NaOH and extracted with EA (500 mL×3). The combined organic layer was washed with brine (300 mL×3), then dried over with anhydrous Na₂SO₄. After filtration, the solution was concentrated under vacuum, and the crude product was purified by Combiflash (PE/EtOAc=2:1) to give the product 1-(5-bromo-1H-pyrrolo [2, 3-b] pyridin-3-yl) ethan-1-one as yellow solid (43.24 g, 71%). Mass (m/z): 241.0 [M+H]⁺.

### Step 2. Preparation of 5-bromo-3-ethyl-1H-pyrrolo [2, 3-b] pyridine

To a solution of AlCl₃ (27.8 g, 0.20 mol) in DME (200 mL) was added LiAlH₄ (4.39 g, 0.1 mol) and 1-(5-bromo-1H-pyrrolo [2, 3-b] pyridin-3-yl) ethan-1-one (10 g, 0.04 mol) at 0° C. The reaction mixture was stirred at rt under N₂ for 3 hs. After the reaction completed, H₂O (500 mL) was added to the reaction mixture, and then extracted with EA (200 mL×3). The combined organic layer was washed with brine (100 mL×2), then dried over with anhydrous Na₂SO₄. The reaction mixture was filtered, the filtrate was concentrated under vacuum to afford compound product 5-bromo-3-ethyl-1H-pyrrolo [2, 3-b] pyridine as yellow solid (11.5 g, 74%). Mass (m/z): 225.0 [M+H]⁺.

### Step 3. Preparation of 5-bromo-3-ethyl-1H-pyrrolo [2, 3-b] pyridine 7-oxide

To a solution of 5-bromo-3-ethyl-1H-pyrrolo [2, 3-b] pyridine (25 g, 0.11 mol) in EA (100 mL) was added 3-Chloroperoxybenzoic acid (26.84 g, 0.155 mol). The reaction mixture was stirred at RT for 3 hrs. The solution was washed with sat. Na₂CO₃ (20 mL) and brine (20 mL), then dried over with anhydrous Na₂SO₄. The reaction mixture was filtered, the filtrate was concentrated to dryness to give the desired product as a white solid (17.4 g, yield: 64.6%). Mass (m/z): 240.7 [M+H]⁺.

### Step 4. Preparation of 5-bromo-4-chloro-3-ethyl-1H-pyrrolo [2, 3-b] pyridine

To a solution of 5-bromo-3-ethyl-1H-pyrrolo [2, 3-b] pyridine 7-oxide (17.3 g, 71.8 mmol) in NMP (15 mL) was added phosphoryl trichloride (55.05 g, 35.9 mmol) at 0° C. C. The reaction mixture was stirred at rt for 16 hrs. The mixture was quenched with water (50 mL), extracted with EA (30 mL×3), washed with sat brine, filtrated, concentrated, the residue was purified by flash column (PE/EA=5:1) to give the desired product as a white solid (4.1 g, yield: 22%). Mass (m/z): 258.7 [M+H]⁺.

### Intermediate A2: 5-bromo-4-chloro-3-cyclopropyl-1H-pyrrolo [2, 3-b] pyridine

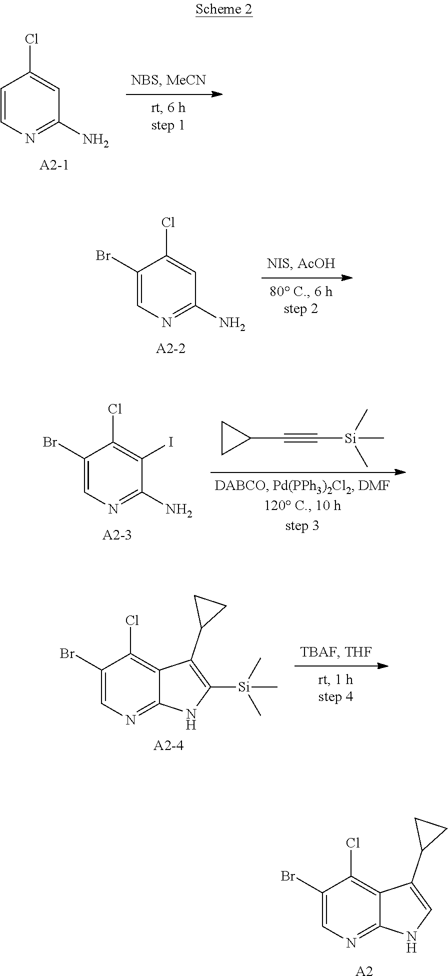

### Step 1. Preparation of 5-bromo-4-chloropyridin-2-amine

To a solution of compound 4-chloropyridin-2-amine (300 g, 2.34 mol, 1.0 eq) in acetonitrile (3000 mL) was added NBS (458 g, 2.57 mol, 1.1 eq) in several portions. The reaction mixture was stirred at room temperature for 6 hrs. Then the reaction was poured into water, filtered. The filter cake was washed with PE and dried to afford compound 5-bromo-4-chloropyridin-2-amine (407 g, 83.9% yield) as a yellow solid. Mass (m/z): 207 [M+H]⁺. ¹HNMR (400 MHz, DMSO-d₆) δ 8.10 (s, 1H), 6.67 (s, 1H), 6.45 (s, 2H).

### Step 2. Preparation of 5-bromo-4-chloro-3-iodopyridin-2-amine

To a solution of compound 5-bromo-4-chloropyridin-2-amine (407 g, 1.97 mol, 1.0 eq) in AcOH (2000 mL) was added NIS (666 g, 2.96 mol, 1.5 eq) in several portions. The reaction mixture was stirred at 80° C. for 4 hrs. The reaction was cooled to room temperature, poured into ice water (5000 mL), adjusted PH>7 with K₂CO₃, extracted with EA (5000 mL×3), washed with a solution of Na₂SO₃ (5000 mL) and brine (5000 mL). The organic phase was concentrated in vacuo to afford compound 5-bromo-4-chloro-3-iodopyridin-2-amine (500 g, 76.3% yield) as a yellow solid. Mass (m/z): 332.7 [M+H]⁺. ¹HNMR (400 MHz, DMSO-d₆) δ 8.10 (s, 1H), 6.62 (s, 2H).

### Step 3. Preparation of 5-bromo-4-chloro-3-cyclopropyl-2-(trimethylsilyl)-1H-pyrrolo [2, 3-b] pyridine

To a solution of compound 5-bromo-4-chloro-3-iodopyridin-2-amine (100 g, 0.300 mol, 1.0 eq), DABCO (101 g, 0.900 mol, 3.0 eq) in DMF (2000 mL) under N₂ was added Pd(PPh₃)₂Cl₂ (21.1 g, 0.03 mol, 0.1 eq). Then compound (cyclopropylethynyl) trimethylsilane (166 g, 1.20 mol, 4.0 eq) was added. The reaction was degassed for 3 times under N₂. The reaction mixture was stirred at 120° C. for 10 hrs. The reaction was filtered, quenched with water (2000 mL), extracted with EA (2000 mL×3), washed with brine (2000 mL), dried over Na₂SO₄, filtered, concentrated in vacuo. The crude was purified by chromatography on sili-gel with THF/PE (1:15) to afford compound 5-bromo-4-chloro-3-cyclopropyl-2-(trimethylsilyl)-1H-pyrrolo [2, 3-b] pyridine (27 g, 26.2% yield) as a yellow solid. Mass (m/z): 344.9 [M+H]⁺.

### Step 4. Preparation of 5-bromo-4-chloro-3-cyclopropyl-1H-pyrrolo [2, 3-b] pyridine

To a mixture of compound 5-bromo-4-chloro-3-cyclopropyl-2-(trimethylsilyl)-1H-pyrrolo [2, 3-b] pyridine (27 g, 79.0 mmol, 1.0 eq) in THF (237 mL) was added TBAF in THF (1.0 M, 237 mL, 3.0 eq) and H₂O (4.27 g, 237 mmol, 3.0 eq). The reaction mixture was stirred at room temperature for 1 hrs. The reaction was quenched with water (1000 mL), extracted with EA (1000 mL×3), washed with brine (1000 mL), dried over Na₂SO₄, filtered, concentrated in vacuo. The crude was purified by chromatography on sili-gel with THF/PE (1:4) to afford the product compound 5-bromo-4-chloro-3-cyclopropyl-1H-pyrrolo [2, 3-b] pyridine (15 g, 70.4% yield) as a faint yellow solid. Mass (m/z): 272.9 [M+H]⁺. ¹HNMR (400 MHz, DMSO-d₆) δ 11.92 (s, 1H), 8.36 (s, 1H), 7.33-7.34 (d, J=4.0 Hz, 1H), 2.11-2.16 (m, 1H), 0.84-0.86 (m, 2H), 0.62-0.64 (m, 2H).

### Intermediate A3: 5-bromo-4-chloro-3-(2, 2-difluoroethyl)-1H-pyrrolo [2, 3-b]pyridine

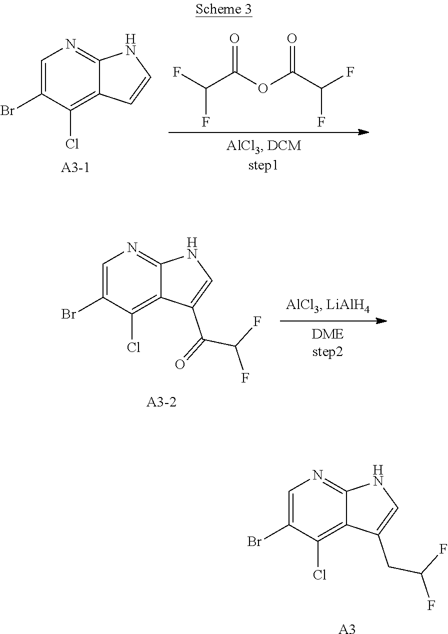

### Step 1. Preparation of 1-{5-bromo-4-chloro-1H-pyrrolo [2, 3-b] pyridin-3-yl}-2, 2-difluoroethanone

To a solution of 3-bromo-4-chloro-1H-pyrrolo [2, 3-b] pyridine (500 mg, 2.16 mol) in DCM (10 mL) was added AlCl₃ (863.78 mg, 6.48 mmol) and 2, 2-difluoroacetyl 2, 2-difluoroacetate (751.9 mg, 4.32 mol) at 0° C. The reaction mixture was stirred at 25° C. under N₂ for 7 hrs. MeOH (30 mL) was added to the reaction mixture and the solvent was removed under reduced pressure. The residue was adjusted to pH 6-7 with 3 N aqueous NaOH and extracted with EA (100 mL×3). The combined organic layers were washed with brine (30 mL×3), then dried over Na₂SO₄. After filtration, the filtrate was concentrated under vacuum, the residue was purified by Combiflash (eluting with PE/EtOAc=2:1) to give the product as yellow solid (200 mg, 11.7%). Mass (m/z): 308.7 [M+H]⁺.

### Step 2. Preparation of 3-bromo-4-chloro-3-(2, 2-difluoroethyl)-1H-pyrrolo [2, 3-b]pyridine

To a solution of AlCl₃ (200 mg, 0.65 mmol) in DME (10 mL) was added LiAlH₄ (64.62 mg, 1.62 mmol) and 1-{5-bromo-4-chloro-1H-pyrrolo [2, 3-b] pyridin-3-yl}-2, 2-difluoroethanone (430.69 g, 3.23 mmol) at 0° C. The reaction mixture was stirred at 25° C. under N₂ for 3 hrs. After the reaction completed, H₂O (100 mL) was added, then extracted with EA (20 mL×3). The combined organic layer was washed with brine (10 mL×2), then dried over Na₂SO₄. The reaction mixture was filtered, the filtrate was concentrated under vacuum and purified by combi-flash, eluting with PE/EA (1:1) to afford compound product 3-bromo-4-chloro-3-(2, 2-difluoroethyl)-1H-pyrrolo [2, 3-b] pyridine (50 mg, 13.1%) as a brown solid compound. Mass (m/z): 295.0 [M+H]+.

### Intermediate A4: 2-(3-(3-(4-chloro-3-ethyl-1H-pyrrolo [2, 3-b] pyridin-5-yl) phenyl)-2-oxotetrahydropyrimidin-1 (2H)-yl) acetaldehyde

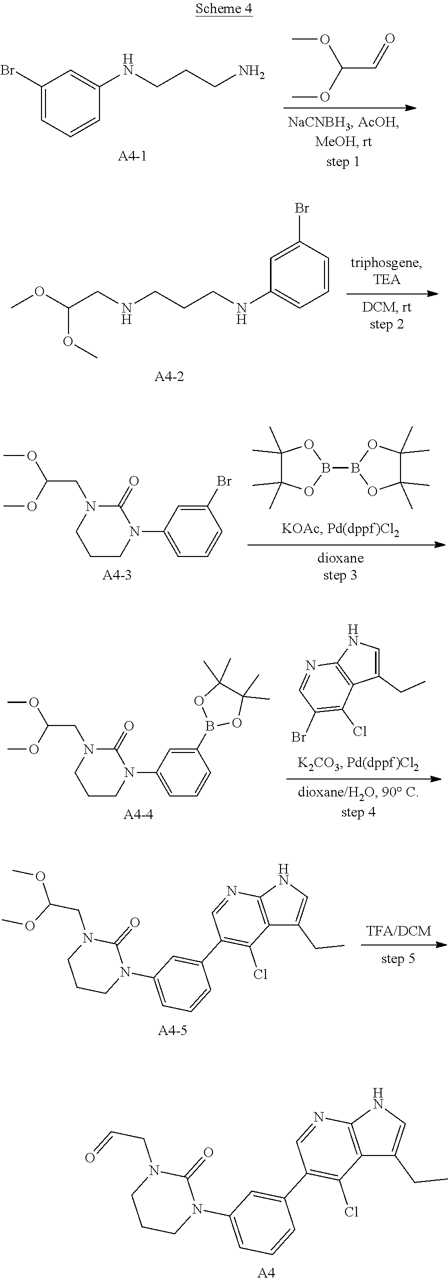

### Step 1. Preparation of {3-[(3-bromophenyl) amino] propyl} (2, 2-dimethoxyethyl) amine

A solution of N-(3-aminopropyl)-3-bromoaniline (5.8 g, 25.3 mmol) and 2, 2-dimethoxyacetaldehyde (2.9 g, 27.8 mmol) in MeOH (100 mL) and AcOH (1 mL) was stirred under nitrogen at 25° C. for 0.5 hr. Then NaBH₃CN (4.8 g, 75.9 mmol) was added at 0° C. The reaction mixture was stirred at 25° C. for 16 hrs. The solution was concentrated under reduced pressure. The residue was purified by flash column (DCM/MeOH=30:1) to give the product as light yellow oil (4.2 g, 47%). Mass (m/z): 317.2 [M+H]⁺.

### Step 2. Preparation of 1-(3-bromophenyl)-3-(2, 2-dimethoxyethyl)-1, 3-diazinan-2-one

A solution of triphosgene (1.53 g, 5.16 mmol) in DCM (20 mL) was added dropwise to a stirred solution of {3-[(3-bromophenyl) amino] propyl} (2, 2-dimethoxyethyl) amine (4.1 g, 12.9 mmol) and DIEA (3.33 g, 25.8 mmol) in DCM (40 mL) at 0° C. over 30 min. The solution was stirred at 25° C. for 2 hrs. The solution was concentrated under reduced pressure. The residue was purified by flash column (PE/EA=1:1) to give the product as light yellow oil (1.8 g, 36%). Mass (m/z): 343.1 [M+H]⁺.

### Step 3. Preparation of 1-(2, 2-dimethoxyethyl)-3-[3-(4, 4, 5, 5-tetramethyl-1, 3, 2-dioxaborolan-2-yl) phenyl]-1, 3-diazinan-2-one

To a solution of 1-(3-bromophenyl)-3-(2, 2-dimethoxyethyl)-1, 3-diazinan-2-one (1.5 g, 4.4 mmol), 4, 4, 5, 5-tetramethyl-2-(4, 4, 5, 5-tetramethyl-1, 3, 2-dioxaborolan-2-yl)-1, 3, 2-dioxaborolane (3.13 g, 12.3 mmol) and KOAc (1.3 g, 13.2 mmol) in 1.4-dioxane (50 mL) was added Pd(dppf)Cl₂ (320 mg, 0.44 mmol). The mixture was stirred under nitrogen at 90° C. for 5 hrs. Water (100 mL) was added and the mixture was extracted with EA (100 mL×3). The combined organic layers were washed with brine (100 mL×2), dried over Na₂SO₄. Then by filtration, the filtrate was concentrated. The residue was purified by column chromatography (PE/EA=1:1) to give the product as brown oil (1.1 g, 57%). Mass (m/z): 391.3 [M+H]⁺.

### Step 4. Preparation of 1-(3-{4-chloro-3-ethyl-1H-pyrrolo [2, 3-b] pyridin-3-yl}phenyl)-3-(2, 2-dimethoxyethyl)-1, 3-diazinan-2-one

To a solution of 1-(2, 2-dimethoxyethyl)-3-[3-(4, 4, 5, 5-tetramethyl-1, 3, 2-dioxaborolan-2-yl) phenyl]-1, 3-diazinan-2-one (200 mg, 0.51 mmol), 3-bromo-4-chloro-3-ethyl-1H-pyrrolo [2, 3-b] pyridine (146 mg, 0.56 mmol) and K₂CO₃ (212 mg, 1.53 mmol) in 1.4-dioxane/H₂O (10:1, 10 mL) was added Pd(dppf)Cl₂ (37 mg, 0.051 mmol). The solution was stirred under nitrogen at 90° C. for 6 hrs. Water (15 mL) was added and the mixture was extracted with EA (10 mL×3). The combined organic layers were washed with brine (20 mL×2), dried over Na₂SO₄. Then by filtration, the filtrate was concentrated. The residue was purified by column chromatography (PE/EA=1:1) to give the product as yellow solid compound (200 mg, 79% yield). Mass (m/z): 443.2 [M+H]+.

### Step 5. Preparation of 2-[3-(3-{4-chloro-3-ethyl-1H-pyrrolo [2, 3-b] pyridin-3-yl}phenyl)-2-oxo-1, 3-diazinan-1-yl] acetaldehyde

To a solution of 1-(3-{4-chloro-3-ethyl-1H-pyrrolo [2, 3-b] pyridin-3-yl} phenyl)-3-(2, 2-dimethoxyethyl)-1, 3-diazinan-2-one (180 mg, 0.41 mmol) in DCM (20 mL) was added TFA (1 mL) and H₂O (1 mL). The reaction mixture was stirred at 25° C. for 16 hrs. Water (15 mL) was added and the mixture was extracted with DCM (20 mL×2). The combined DCM layers were washed with saturated NaHCO₃ (15 mL×2) and brine (20 mL), dried over Na₂SO₄ and concentrated to give the product as brown oil (220 mg, 82%). Mass (m/z): 397.2 [M+H]⁺.

### Intermediate A5: tert-butyl 4-(2-(2, 6-dioxopiperidin-3-yl)-1, 3-dioxoisoindolin-5-yl)-3, 6-dihydropyridine-1 (2H)-carboxylate

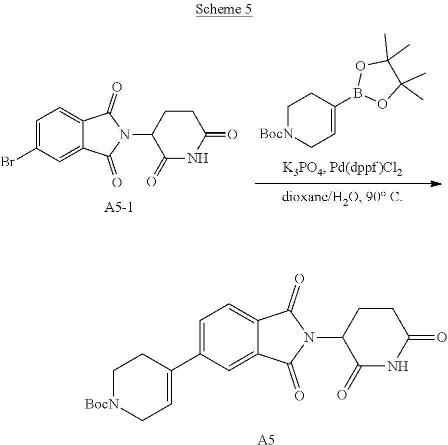 [SMILES: O=C1CCC(N2C(=O)c3ccc(Br)cc3C2=O)C(=O)N1 ; CC(C)(C)OC(=O)N1CC=C(B2OC(C)(C)C(C)(C)O2)CC1 ; CC(C)(C)OC(=O)N1CC=C(c2ccc3c(c2)C(=O)N(C2CCC(=O)NC2=O)C3=O)CC1 ; "A5"]

To a mixture of 5-bromo-2-(2, 6-dioxopiperidin-3-yl) isoindole-1, 3-dione (1 g, 3 mmol), tert-butyl [4-(4, 4, 5, 5-tetramethyl-1, 3, 2-dioxaborolan-2-yl)-3, 6-dihydro-2H-pyridin-1-yl] formate (1.12 g, 3.6 mmol) and K₃PO₄ (0.76 g, 3.6 mmol) in dioxane/H₂O (10:1, 20 mL) was added Pd(dppf)Cl₂ (0.12 g, 0.1 mmol). The reaction mixture was stirred at 90° C. under N₂ for 18 hrs. Water (20 mL) was added and the mixture was extracted with DCM (20 mL×2). The organic layer was washed with brine (20 mL×2), dried over Na₂SO₄ and concentrated. The residue was purified by combi-flash with (DCM/MeOH=10:1) to give the product (1 g, 73%) as a brown solid. Mass (m/z): 462.1 [M+Na]⁺.

### Intermediate A6: tert-butyl 4-(5-(2, 4-dioxotetrahydropyrimidin-1 (2H)-yl) naphthalen-2-yl) piperazine-1-carboxylate

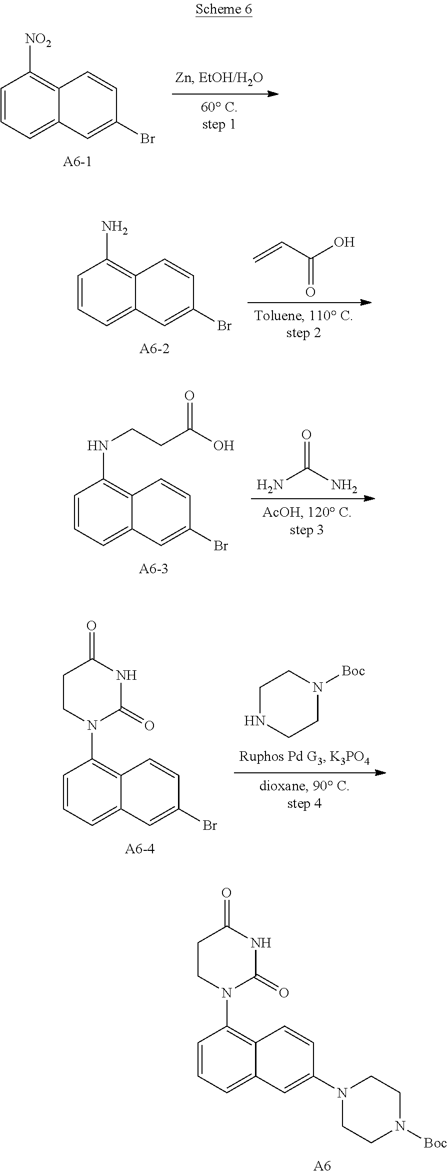

### Step 1. Preparation of 6-bromonaphthalen-1-amine

To a mixture of 6-bromo-1-nitronaphthalene (5.00 g, 0.0198 mol) in EtOH/H₂O (3:1, 50 mL) was added NH₄Cl (7.73 g, 0.145 mol). The reaction mixture was warmed to 60° C. and then Zn powder (9.45 g, 0.145 mol) was added portion wise. The reaction mixture was stirred at 60° C. for 1 hr. The reaction mixture was filtered, and the filtrate was concentrated under reduced pressure. The residue was diluted with water (200 mL) then extracted with EtOAc (150 mL×3), washed with brine (200 mL), dried over Na₂SO₄ and concentrated under reduced pressure. The crude product was purified by Flash Chromatography (PE/EA=0˜40%) to give the product 6-bromonaphthalen-1-amine as a brown solid (3.87 g, 83%). Mass (m/z): 221.9 [M+H]⁺.

### Step 2. Preparation of 3-[(6-bromonaphthalen-1-yl) amino] propanoic acid

To a mixture of 6-bromonaphthalen-1-amine (3.87 g, 0.0174 mol) in toluene (40.0 mL) was added prop-enoic acid (7.52 g, 0.104 mol). The reaction was degassed with N₂ and stirred at 110° C. for 21 hrs. The reaction mixture was concentrated under reduce pressure to the product 3-[(6-bromonaphthalen-1-yl) amino] propanoic acid as a brown solid (8.00 g, 93%). Mass (m/z): 293.9 [M+H]⁺.

### Step 3. Preparation of 1-(6-bromonaphthalen-1-yl)-1, 3-diazinane-2, 4-dione

To a mixture of 3-[(6-bromonaphthalen-1-yl) amino] propanoic acid (8.7 g, 0.0296 mol) in AcOH (180 mL) was added urea (4.44 g, 0.0740 mol). The reaction mixture was stirred at 120° C. under N₂ for 16 hrs. The reaction mixture was poured into water (200 mL) slowly at 0° C. and then filtered. The cake was dried under reduced pressure to give the product 1-(6-bromonaphthalen-1-yl)-1, 3-diazinane-2, 4-dione as brown solid (7.5 g, 71%). Mass (m/z): 318.8 [M+H]⁺.

### Step 4. Preparation of tert-butyl {4-[5-(2, 4-dioxo-1, 3-diazinan-1-yl) naphthalen-2-yl] piperazin-1-yl} formate

To a mixture of 1-(6-bromonaphthalen-1-yl)-1, 3-diazinane-2, 4-dione (500 mg, 1.57 mmol) in dioxane (10.0 mL) was added K₃PO₄ (998 mg, 4.70 mmol), tert-butyl piperazin-1-yl-formate (586 mg, 3.133 mmol) and RuPhos Pd G3 (262 mg, 0.313 mmol). The reaction was degassed with N₂ for 3 times and stirred at 90° C. for 16 hrs. The reaction mixture was filtered and the filtrate was concentrated under reduced pressure. The residue was diluted with EA (25 mL) and H₂O (50 mL), then extracted with EA (50 mL×3), washed with brine (100 mL), dried over Na₂SO₄ and concentrated under reduced pressure. The crude product was purified by Flash Chromatography (PE/EA=0˜30%) to give the product tert-butyl {4-[5-(2, 4-dioxo-1, 3-diazinan-1-yl) naphthalen-2-yl] piperazin-1-yl} formate as a white solid (360 mg, 49%). Mass (m/z): 424.9 [M+H]⁺.

Intermediates A7 and A8: 3-{1-oxo-6-[4-({2-oxo-3-[3-(4, 4, 5, 5-tetramethyl-1, 3, 2-dioxaborolan-2-yl) phenyl]-1, 3-diazinan-1-yl} methyl) piperidin-1-yl]-3H-isoindol-2-yl} piperidine-2, 6-dione and 3-(1-oxo-5-(4-((2-oxo-3-(3-(4, 4, 5, 5-tetramethyl-1, 3, 2-dioxaborolan-2-yl) phenyl) tetrahydropyrimidin-1 (2H)-yl) methyl) piperidin-1-yl) isoindolin-2-yl) piperidine-2, 6-dione

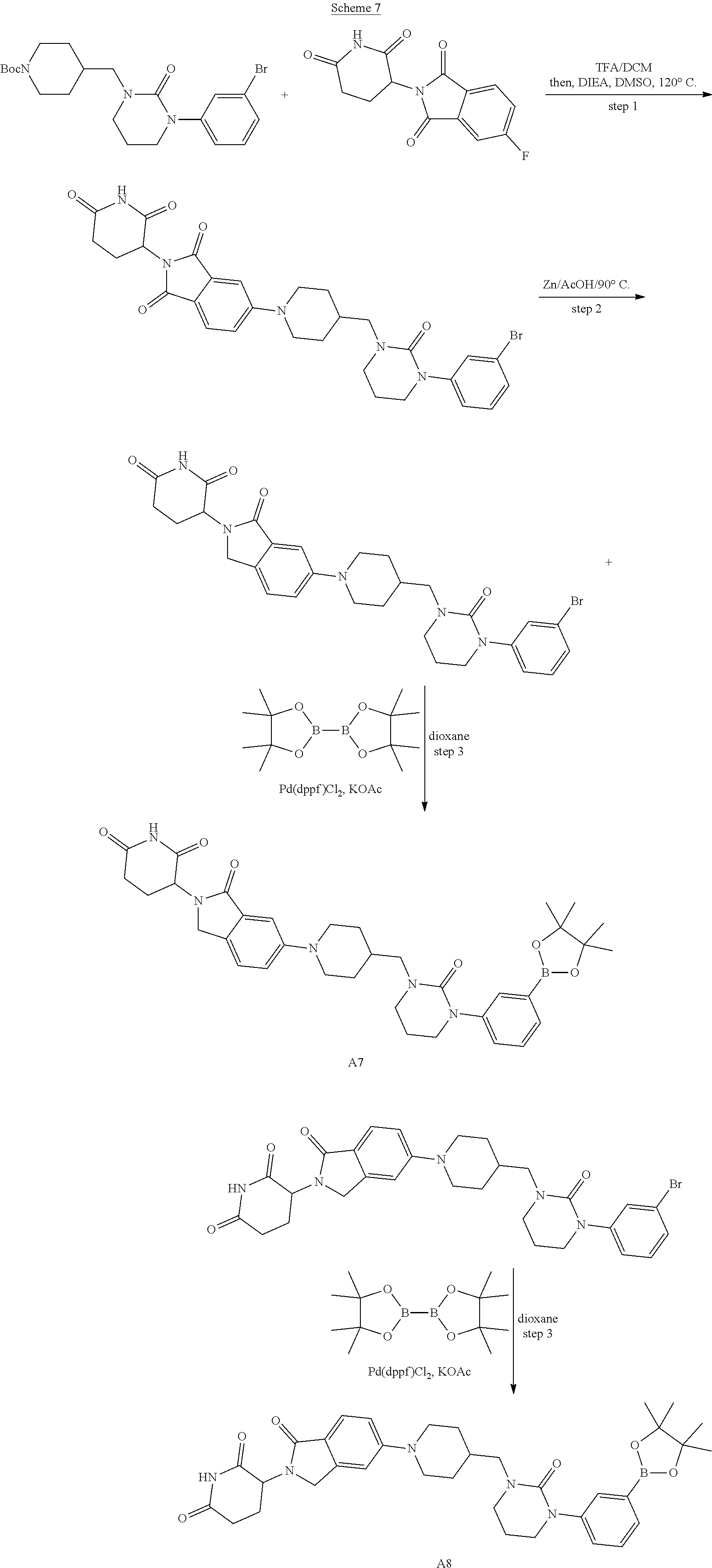

### Step 1. Preparation of 5-(4-{[3-(3-bromophenyl)-2-oxo-1, 3-diazinan-1-yl]methyl} piperidin-1-yl)-2-(2, 6-dioxopiperidin-3-yl) isoindole-1, 3-dione

To a solution of tert-butyl 4-((3-(3-bromophenyl)-2-oxotetrahydropyrimidin-1 (2H)-yl) methyl) piperidine-1-carboxylate (1.3 g, 2.8 mmol) in DCM (40 mL) was added TFA (2 mL) and H₂O (2 mL). The reaction mixture was stirred at 25° C. for 16 hrs. Water (30 mL) was added and the mixture was extracted with DCM (40 mL×2). The combined DCM layers were washed with saturated NaHCO₃ (30 mL×2) and brine (40 mL), dried over Na₂SO₄ and concentrated to give crude product. A solution of this crude product, 1-(3-bromophenyl)-3-(piperidin-4-ylmethyl)-1, 3-diazinan-2-one (1 g, 2.8 mmol) and DIEA (1.09 g, 8.4 mmol) in DMSO (20 mL) was stirred under nitrogen at 120° C. for 2 hrs. Water (50 mL) was added and the mixture was extracted with EA (30 mL×3). The combined organic layers were washed with brine (50 mL×3), dried over Na₂SO₄. Then by filtration, the filtrate was concentrated. The residue was purified by flash Chromatography (PE/EA=1:1) to give the product as a brown solid (1.1 g, 57%). Mass (m/z): 607.7 [M+H]⁺.

### Step 2. Preparation of 3-[5-(4-{[3-(3-bromophenyl)-2-oxo-1, 3-diazinan-1-yl]methyl} piperidin-1-yl)-1-oxo-3H-isoindol-2-yl] piperidine-2, 6-dione and 3-(5-(4-((3-(3-bromophenyl)-2-oxotetrahydropyrimidin-1 (2H)-yl) methyl) piperidin-1-yl)-1-oxoisoindolin-2-yl) piperidine-2, 6-dione

To a solution of 5-(4-{[3-(3-bromophenyl)-2-oxo-1, 3-diazinan-1-yl] methyl}piperidin-1-yl)-2-(2, 6-dioxopiperidin-3-yl) isoindole-1, 3-dione (1.4 g, 2.3 mmol) in AcOH (20 mL) was added Zn powder (1.5 g, 2.3 mmol), the reaction mixture was stirred at 90° C. for 16 hrs. The reaction mixture was filtered and the filtrate was concentrated. Water (30 mL) was added and the mixture was extracted with DCM (30 mL×3). The combined organic layers were washed with brine (20 mL×2), dried over Na₂SO₄. Then by filtration, the filtrate was concentrated. The residue was purified by Prep-HPLC [Gemini-C18, 150×21.2 mm, 5 um; ACN-H₂O (0.1% TFA), 55-60] to give the products:

3-[5-(4-{[3-(3-bromophenyl)-2-oxo-1, 3-diazinan-1-yl] methyl} piperidin-1-yl)-1-oxo-3H-isoindol-2-yl] piperidine-2, 6-dione (430 mg, 27%). Mass (m/z): 680.3 [M+H]⁺.

3-(5-(4-((3-(3-bromophenyl)-2-oxotetrahydropyrimidin-1 (2H)-yl) methyl) piperidin-1-yl)-1-oxoisoindolin-2-yl) piperidine-2, 6-dione (180 mg, 11%). Mass (m/z): 680.3 [M+H]⁺.

### Step 3. Preparation of 3-{1-oxo-6-[4-({2-oxo-3-[3-(4, 4, 5, 5-tetramethyl-1, 3, 2-dioxaborolan-2-yl) phenyl]-1, 3-diazinan-1-yl} methyl) piperidin-1-yl]-3H-isoindol-2-yl} piperidine-2, 6-dione and 3-(1-oxo-5-(4-((2-oxo-3-(3-(4, 4, 5, 5-tetramethyl-1, 3, 2-dioxaborolan-2-yl) phenyl) tetrahydropyrimidin-1 (2H)-yl) methyl) piperidin-1-yl) isoindolin-2-yl) piperidine-2, 6-dione

To a solution of 3-[6-(4-{[3-(3-bromophenyl)-2-oxo-1, 3-diazinan-1-yl] methyl}piperidin-1-yl)-1-oxo-3H-isoindol-2-yl] piperidine-2, 6-dione (430 mg, 0.72 mmol), 4, 4, 5, 5-tetramethyl-2-(4, 4, 5, 5-tetramethyl-1, 3, 2-dioxaborolan-2-yl)-1, 3, 2-dioxaborolane (275 mg, 1.08 mmol) and KOAc (213 mg, 2.17 mmol) in 1.4-dioxane (10 mL) was added Pd(dppf)Cl₂ (53 mg, 0.072 mmol). The mixture was stirred under nitrogen at 110° C. for 2 hrs. Water (15 mL) was added and the mixture was extracted with EA (10 mL×3). The combined organic layers were washed with brine (20 mL×2), dried over Na₂SO₄. Then by filtration, the filtrate was concentrated. The residue was purified by flash Chromatography (DCM/MeOH=20:1) to give the product A7 as a brown solid (300 mg, 58%). Mass (m/z): 642.4 [M+H]⁺.

Following the same procedure from 3-(5-(4-((3-(3-bromophenyl)-2-oxotetrahydropyrimidin-1 (2H)-yl) methyl) piperidin-1-yl)-1-oxoisoindolin-2-yl) piperidine-2, 6-dione, product A8 was obtained as a brown solid (140 mg, 64%). Mass (m/z): 642.3 [M+H]⁺.

### General Synthesis Procedure I:

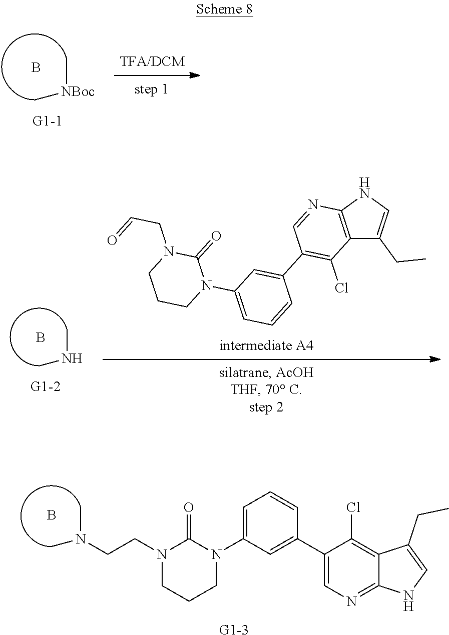

### Step 1. Preparation G1-2

To a solution of G1-1 (0.25 mmol) in DCM (10 mL) was added TFA (1 mL). The reaction mixture was stirred at rt for 3 hrs. Methyl tertbutyl ether (50 mL) was added and the mixture was filtered. The cake was dried to give the product G1-2, which was used directly for next step.

### Step 2. Preparation of G1-3

To a solution of 2-[3-(3-{4-chloro-3-ethyl-1H-pyrrolo [2, 3-b] pyridin-3-yl}phenyl)-2-oxo-1, 3-diazinan-1-yl] acetaldehyde (110 mg, 0.28 mmol) in THF (3 mL) was added G1-2 (0.26 mmol), silatrane (146 mg, 0.83 mmol) and AcOH (one drop). The reaction mixture was stirred at 70° C. for 16 hrs. The solution was concentrated under reduced pressure. The residue was purified by Prep-HPLC [Gemini-C18, 150×21.2 mm, Sum; ACN-H₂O (0.1% FA), 30-40] to give the product.

General synthesis procedure II:

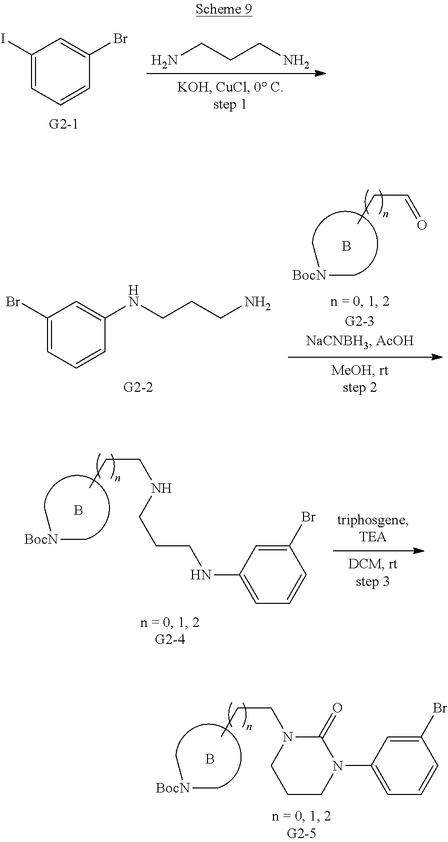

### Step 1. Preparation of G2-2

To a solution of 1, 3-dibromobenzene (18 g, 63.63 mmol) and propane-1, 3-diamine (14.1 g, 190.87 mmol) and KOH (7.14 g, 127.25 mmol) and CuCl (630 mg, 6.36 mmol), the resulting mixture was stirred at 0° C. under N₂ for 16 hrs. After the reaction completed, H₂O (500 mL) was added to the reaction mixture, and then extracted with DCM (500 mL×3). The combined organic layer was washed with brine (300 mL×2), then dried over anhydrous Na₂SO₄. After filtration, the solution was concentration under vacuum, and the residue was purified by Combiflash [DCM/MeOH (10% NH₃)=0˜10%] to give the product as brown oil (10.7 g, 73%). Mass (m/z): 231.1 [M+H]⁺.

### Step 2. Preparation of G2-4

To a solution of N¹-(3-bromophenyl) propane-1, 3-diamine (10.7 g, 46.96 mmol) in MeOH (200 mL) was added G2-3 (46.96 mmol) and AcOH (0.5 mL) and NaCNBH₃ (8.85 g, 140.88 mmol) at 0° C. The reaction mixture was stirred at rt for 2 hrs. The solvent was removed under reduced pressure and the residue was purified by Combiflash column [DCM/MeOH (10% NH₃)=0˜10%] to give the product.

### Step 3. Preparation of G2-5

To a solution of G2-4 (42.19 mmol) in DCM (200 mL) was added TEA (8.5 g, 84.37 mmol) and triphosgene (6.25 g, 21.09 mmol) at 0° C. The reaction mixture was stirred at rt for 2 hrs. After the reaction completed, H₂O (500 mL) was added to the reaction mixture, and then extracted with DCM (500 mL×3). The combined organic layer was washed with brine (300 mL×2), then dried over anhydrous Na₂SO₄. After filtration, the solution was concentration under vacuum, and the residue was purified by Combiflash (DCM/MeOH=0˜10%) to give the product.

### Example 1

### 3-[6-(1-{2-[3-(3-{4-chloro-3-ethyl-1H-pyrrolo [2, 3-b] pyridin-3-yl} phenyl)-2-oxo-1, 3-diazinan-1-yl] ethyl} azetidin-3-yl)-1-oxo-3H-isoindol-2-yl] piperidine-2, 6-dione

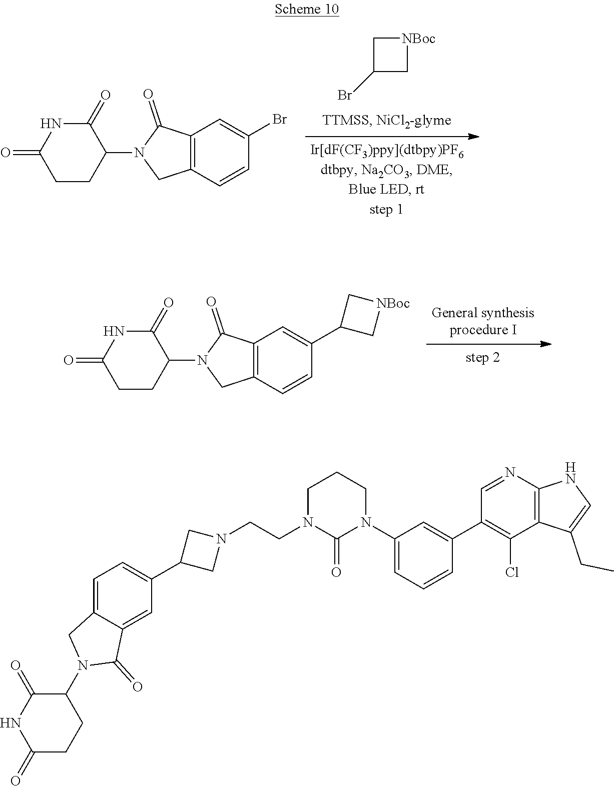 [SMILES: O=C1CCC(N2Cc3ccc(Br)cc3C2=O)C(=O)N1 ; CC(C)(C)OC(=O)N1CC(Br)C1 ; CC(C)(C)OC(=O)N1CC(c2ccc3c(c2)C(=O)N(C2CCC(=O)NC2=O)C3)C1 ; CCc1c[nH]c2ncc(-c3cccc(N4CCCN(CCN5CC(c6ccc7c(c6)C(=O)N(C6CCC(=O)NC6=O)C7)C5)C4=O)c3)c(Cl)c12]

### Step 1. Preparation of tert-butyl 3-(2-(2, 6-dioxopiperidin-3-yl)-3-oxoisoindolin-5-yl) azetidine-1-carboxylate

A solution of 3-(6-bromo-1-oxoisoindolin-2-yl) piperidine-2, 6-dione (626 mg, 2 mmol), tert-butyl 3-bromoazetidine-1-carboxylate (400 mL, 2.4 mmol), TTMSS (750 mL, 2.4 mmol), NiCl₂-glyme (22 mg, 0.1 mmol), dtbpy (27 mg, 0.1 mmol), Ir[dF(CF3)ppy] (dtbpy) PF6 (23 mg, 0.02 mmol), Na₂CO₃ (424 mg, 4 mmol) in DME (10 mL) was stirred under nitrogen at 25° C. and irradiated with a blue LED for 16 hr. The solution was filtered through celite, concentrated under reduced pressure. The residue was purified by flash column (DCM/MeOH=40:1) to give the product as light violet solid (466 mg, 58%). Mass (m/z): 400.2 [M+H]⁺.

### Step 2

Following general synthesis procedure I, from tert-butyl 3-(2-(2, 6-dioxopiperidin-3-yl)-3-oxoisoindolin-5-yl) azetidine-1-carboxylate, the product 3-[6-(1-{2-[3-(3-{4-chloro-3-ethyl-1H-pyrrolo [2, 3-b] pyridin-3-yl} phenyl)-2-oxo-1, 3-diazinan-1-yl] ethyl} azetidin-3-yl)-1-oxo-3H-isoindol-2-yl] piperidine-2, 6-dione was obtained as a yellow solid (17 mg, 8.6%). Mass (m/z): 680.3 [M+H]⁺. ¹H NMR (400 MHz, DMSO-d₆) δ 11.79 (s, 1H), 11.01 (d, J=4.6 Hz, 1H), 9.80-9.53 (m, 1H), 8.11 (d, J=5.8 Hz, 1H), 7.94 (d, J=22.2 Hz, 1H), 7.62 (dd, J=21.4, 7.6 Hz, 2H), 7.42 (dd, J=14.0, 7.2 Hz, 4H), 7.23 (t, J=6.8 Hz, 1H), 5.13 (dd, J=13.2, 4.8 Hz, 1H), 4.48 (d, J=8.6 Hz, 2H), 4.43 (s, 1H), 4.34-4.22 (m, 4H), 3.55 (s, 4H), 3.48 (s, 2H), 3.42 (d, J=5.6 Hz, 2H), 2.96-2.85 (m, 4H), 2.02-1.99 (m, 4H), 1.27 (t, J=7.2 Hz, 3H).

### Example 2

### 3-[5-(1-{2-[3-(3-{4-chloro-3-ethyl-1H-pyrrolo [2, 3-b] pyridin-3-yl} phenyl)-2-oxo-1, 3-diazinan-1-yl] ethyl} azetidin-3-yl)-1-oxo-3H-isoindol-2-yl] piperidine-2, 6-dione

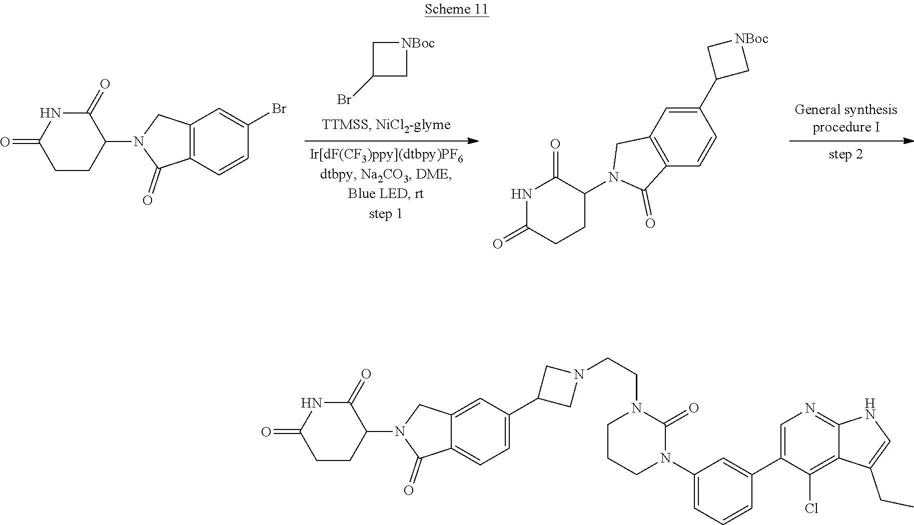 [SMILES: O=C1CCC(N2Cc3cc(Br)ccc3C2=O)C(=O)N1 ; CC(C)(C)OC(=O)N1CC(Br)C1 ; CC(C)(C)OC(=O)N1CC(c2ccc3c(c2)CN(C2CCC(=O)NC2=O)C3=O)C1 ; CCc1c[nH]c2ncc(-c3cccc(N4CCCN(CCN5CC(c6ccc7c(c6)CN(C6CCC(=O)NC6=O)C7=O)C5)C4=O)c3)c(Cl)c12]

### Step 1. Preparation of tert-butyl 3-(2-(2, 6-dioxopiperidin-3-yl)-1-oxoisoindolin-5-yl) azetidine-1-carboxylate

A solution of 3-(5-bromo-1-oxoisoindolin-2-yl) piperidine-2, 6-dione (517 mg, 1.6 mmol), tert-butyl 3-bromoazetidine-1-carboxylate (315 mL, 1.9 mmol), TTMSS (600 mL, 1.9 mmol), NiCl₂-glyme (18 mg, 0.08 mmol), dtbpy (22 mg, 0.08 mmol), Ir[dF(CF3)ppy] (dtbpy) PF6 (18 mg, 0.016 mmol), Na₂CO₃ (339 mg, 3.2 mmol) in DME (8 mL) was stirred under nitrogen at 25° C. and irradiated with a blue LED for 16 hr. The solution was filtered through celite, concentrated under reduced pressure. The residue was purified by flash column (DCM/MeOH=40:1) to give the product as light pink solid (458 mg, 71%). Mass (m/z): 400.2 [M+H]⁺.

### Step 2

Following general synthesis procedure I, from tert-butyl 3-(2-(2, 6-dioxopiperidin-3-yl)-1-oxoisoindolin-5-yl) azetidine-1-carboxylate, the product 3-[5-(1-{2-[3-(3-{4-chloro-3-ethyl-1H-pyrrolo [2, 3-b] pyridin-3-yl} phenyl)-2-oxo-1, 3-diazinan-1-yl] ethyl} azetidin-3-yl)-1-oxo-3H-isoindol-2-yl] piperidine-2, 6-dione was obtained as a yellow solid (13 mg, 6.6%). Mass (m/z): 680.3 [M+H]⁺. ¹H NMR (400 MHz, DMSO-d₆) δ 11.79 (s, 1H), 11.00 (s, 1H), 9.83-9.55 (m, 1H), 8.11 (d, J=4.8 Hz, 1H), 7.73 (d, J=7.8 Hz, 1H), 7.67 (s, 1H), 7.56 (d, J=7.8 Hz, 1H), 7.42 (dd, J=15.8, 9.0 Hz, 4H), 7.24 (d, J=6.6 Hz, 1H), 5.12 (dd, J=13.2, 5.0 Hz, 1H), 4.51 (d, J=5.2 Hz, 2H), 4.41 (d, J=5.8 Hz, 1H), 4.28 (dd, J=29.2, 10.6 Hz, 4H), 3.56 (s, 4H), 3.45 (d, J=17.2 Hz, 2H), 3.42-3.36 (m, 2H), 2.91 (td, J=12.4, 5.4 Hz, 4H), 2.19-1.88 (m, 4H), 1.26 (dd, J=14.4, 7.2 Hz, 3H).

### Example 3

### 5-(1-(2-(3-(3-(4-chloro-3-ethyl-1H-pyrrolo [2, 3-b] pyridin-5-yl) phenyl)-2-oxotetrahydropyrimidin-1 (2H)-yl) ethyl) piperidin-4-yl)-2-(2, 6-dioxopiperidin-3-yl) isoindoline-1, 3-dione

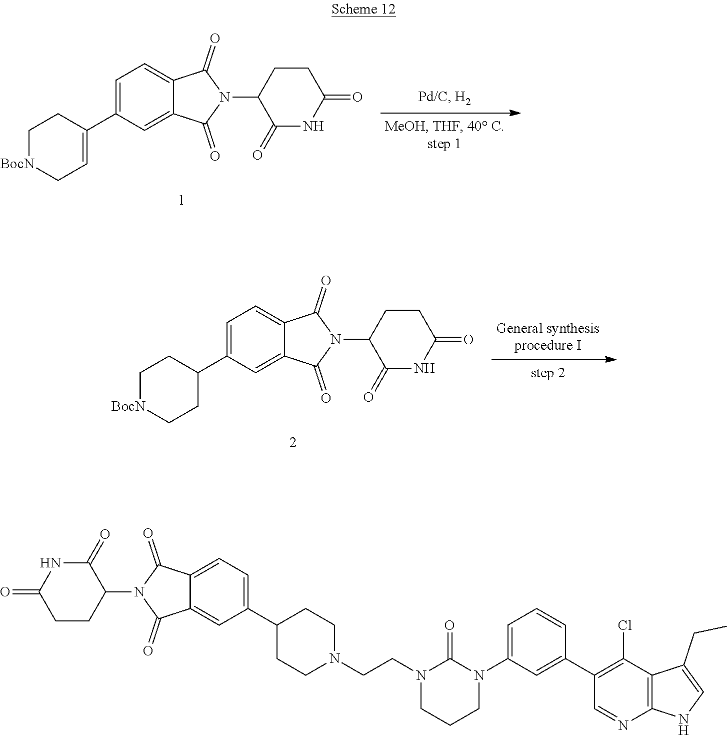 [SMILES: CC(C)(C)OC(=O)N1CC=C(c2ccc3c(c2)C(=O)N(C2CCC(=O)NC2=O)C3=O)CC1 ; "1" ; CC(C)(C)OC(=O)N1CCC(c2ccc3c(c2)C(=O)N(C2CCC(=O)NC2=O)C3=O)CC1 ; "2" ; CCc1c[nH]c2ncc(-c3cccc(N4CCCN(CCN5CCC(c6ccc7c(c6)C(=O)N(C6CCC(=O)NC6=O)C7=O)CC5)C4=O)c3)c(Cl)c12]

### Step 1. Preparation of tert-butyl 4-(2-(2, 6-dioxopiperidin-3-yl)-1, 3-dioxoisoindolin-5-yl) piperidine-1-carboxylate

To a solution of tert-butyl 4-[2-(2, 6-dioxopiperidin-3-yl)-1, 3-dioxoisoindol-5-yl]-3, 6-dihydro-2H-pyridine-1-carboxylate (500 mg, 1.13 mmol) in MeOH (10 mL) and THF (10 mL) was added 10% Pd/C (50 mg, 10% wt/wt). Then the reaction was stirred for 18 h at 40° C. under 1 atm of H₂. The mixture was filtered and the filtrate was concentrated to give the target product (400 mg, 75%) as a gray solid. Mass (m/z) 464.0 [M+Na]⁺.

### Step 2

Following general synthesis procedure I, from tert-butyl 4-(2-(2, 6-dioxopiperidin-3-yl)-1, 3-dioxoisoindolin-5-yl) piperidine-1-carboxylate, the desired product 5-(1-(2-(3-(3-(4-chloro-3-ethyl-1H-pyrrolo [2, 3-b] pyridin-5-yl) phenyl)-2-oxotetrahydropyrimidin-1 (2H)-yl) ethyl) piperidin-4-yl)-2-(2, 6-dioxopiperidin-3-yl) isoindoline-1, 3-dione was obtained (16 mg, 9%) as a white solid. Mass (m/z): 722.0 [M+H]⁺. ¹H NMR (400 MHz, DMSO-d₆) δ 11.73 (s, 1H), 11.09 (s, 1H), 8.09 (s, 1H), 8.06 (s, 1H), 7.80-7.74 (m, 2H), 7.70 (d, J=7.8 Hz, 1H), 7.37-7.32 (m, 3H), 7.30 (d, J=8.0 Hz, 1H), 7.17 (d, J=6.4 Hz, 1H), 5.19-5.04 (m, 1H), 3.69 (d, J=4.8 Hz, 2H), 3.41 (dd, J=14.0, 8.4 Hz, 8H), 2.88-2.82 (m, 3H), 2.56 (d, J=17.8 Hz, 4H), 2.02 (d, J=4.6 Hz, 4H), 1.81 (s, 2H), 1.73 (s, 2H), 1.21 (t, J=7.4 Hz, 3H).

### Example 4

### 1-(6-(4-(2-(3-(3-(4-chloro-3-ethyl-1H-pyrrolo [2, 3-b] pyridin-5-yl) phenyl)-2-oxotetrahydropyrimidin-1 (2H)-yl) ethyl) piperazin-1-yl) naphthalen-1-yl) dihydropyrimidine-2, 4 (1H, 3H)-dione

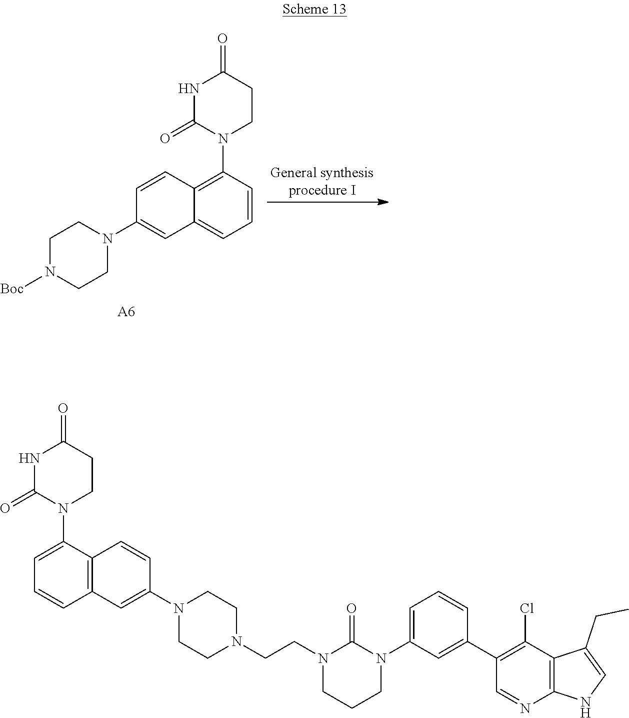

Following general synthesis procedure I, from tert-butyl 4-(5-(2, 4-dioxotetrahydropyrimidin-1 (2H)-yl) naphthalen-2-yl) piperazine-1-carboxylate, the desired product 1-(6-(4-(2-(3-(3-(4-chloro-3-ethyl-1H-pyrrolo [2, 3-b] pyridin-5-yl) phenyl)-2-oxotetrahydropyrimidin-1 (2H)-yl) ethyl) piperazin-1-yl) naphthalen-1-yl) dihydropyrimidine-2, 4(1H, 3H)-dione was obtained (12 mg, 8%) as a white solid. Mass (m/z): 704.8 [M+H]⁺. ¹H NMR (400 MHz, DMSO-d₆) δ 11.78 (s, 1H), 10.43 (s, 1H), 8.13 (d, J=15.2 Hz, 2H), 7.72 (dd, J=18.8, 8.8 Hz, 2H), 7.41 (dd, J=14.6, 7.2 Hz, 4H), 7.33 (d, J=8.0 Hz, 1H), 7.22 (dd, J=14.6, 7.8 Hz, 3H), 3.85 (d, J=4.8 Hz, 1H), 3.76-3.68 (m, 2H), 3.67-3.54 (m, 2H), 3.46 (d, J=5.2 Hz, 4H), 3.26 (s, 3H), 2.91 (dd, J=15.0, 7.6 Hz, 3H), 2.77-2.52 (m, 7H), 2.05 (s, 2H), 1.26 (t, J=7.4 Hz, 3H).

### Example 5

### 5-(1-(2-(3-(3-(4-chloro-3-ethyl-1H-pyrrolo [2, 3-b] pyridin-5-yl) phenyl)-2-oxotetrahydropyrimidin-1 (2H)-yl) ethyl)-1, 2, 3, 6-tetrahydropyridin-4-yl)-2-(2, 6-dioxopiperidin-3-yl) isoindoline-1, 3-dione

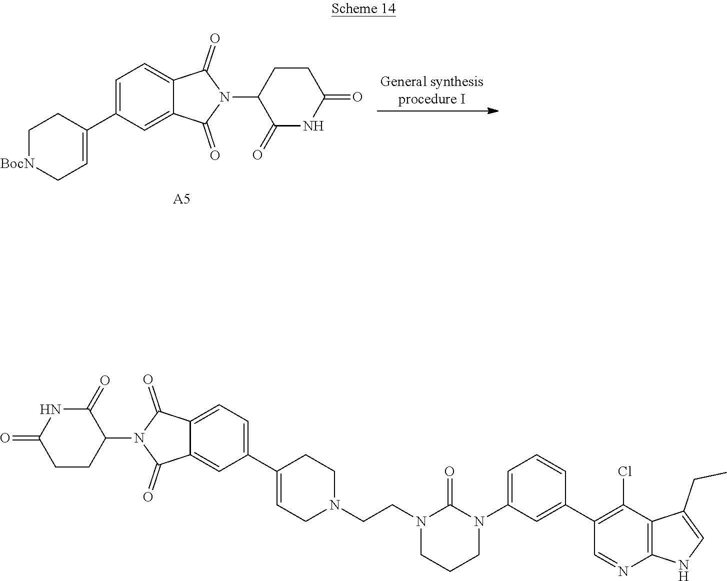 [SMILES: CC(C)(C)OC(=O)N1CC=C(c2ccc3c(c2)C(=O)N(C2CCC(=O)NC2=O)C3=O)CC1 ; "A5" ; CCc1c[nH]c2ncc(-c3cccc(N4CCCN(CCN5CC=C(c6ccc7c(c6)C(=O)N(C6CCC(=O)NC6=O)C7=O)CC5)C4=O)c3)c(Cl)c12]

Following general synthesis procedure I, from tert-butyl 4-(2-(2, 6-dioxopiperidin-3-yl)-1, 3-dioxoisoindolin-5-yl)-3, 6-dihydropyridine-1 (2H)-carboxylate, the desired product 5-(1-(2-(3-(3-(4-chloro-3-ethyl-1H-pyrrolo [2, 3-b] pyridin-5-yl) phenyl)-2-oxotetrahydropyrimidin-1 (2H)-yl) ethyl)-1, 2, 3, 6-tetrahydropyridin-4-yl)-2-(2, 6-dioxopiperidin-3-yl) isoindoline-1, 3-dione was obtained (31 mg, 17%) as a white solid. Mass (m/z): 720.0 [M+H]⁺. ¹H NMR (400 MHz, DMSO-d₆) δ 11.78 (d, J=2.2 Hz, 1H), 11.15 (s, 1H), 8.10 (s, 1H), 7.98 (s, 1H), 7.89 (dd, J=18.0, 7.8 Hz, 2H), 7.38 (tt, J=16.0, 8.0 Hz, 4H), 7.22 (d, J=7.6 Hz, 1H), 6.56 (s, 1H), 5.16 (dd, J=12.8, 5.2 Hz, 1H), 3.73 (s, 4H), 3.58 (s, 2H), 3.45 (t, J=5.8 Hz, 4H), 2.97-2.82 (m, 4H), 2.80-2.52 (m, 5H), 2.13-1.95 (m, 3H), 1.26 (t, J=7.4 Hz, 3H).

### Example 6

### 5-(1-(2-(3-(3-(4-chloro-3-ethyl-1H-pyrrolo [2, 3-b] pyridin-5-yl) phenyl)-2-oxotetra hydropyrimidin-1 (2H)-yl) ethyl) azetidin-3-yl)-2-(2, 6-dioxopiperidin-3-yl) isoindo line-1, 3-dione

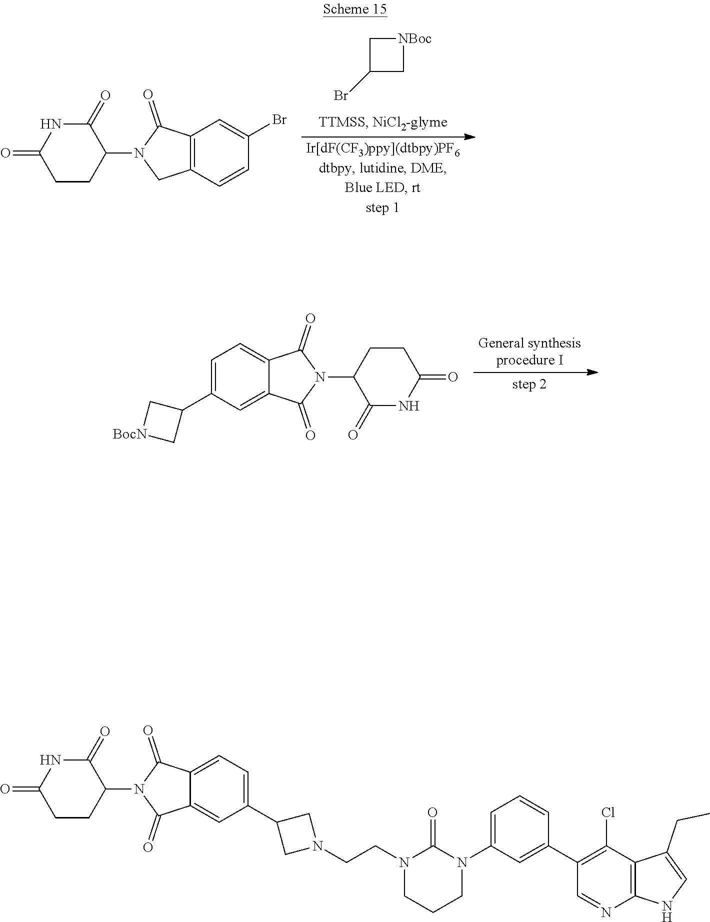 [SMILES: O=C1CCC(N2Cc3ccc(Br)cc3C2=O)C(=O)N1 ; CC(C)(C)OC(=O)N1CC(Br)C1 ; CC(C)(C)OC(=O)N1CC(c2ccc3c(c2)C(=O)N(C2CCC(=O)NC2=O)C3=O)C1 ; CCc1c[nH]c2ncc(-c3cccc(N4CCCN(CCN5CC(c6ccc7c(c6)C(=O)N(C6CCC(=O)NC6=O)C7=O)C5)C4=O)c3)c(Cl)c12]

### Step 1. Preparation of tert-butyl 3-(2-(2, 6-dioxopiperidin-3-yl)-1, 3-dioxoisoindolin-5-yl) azetidine-1-carboxylate

A solution of 5-bromo-2-(2, 6-dioxopiperidin-3-yl) isoindoline-1, 3-dione (337 mg, 1 mmol), tert-butyl 3-bromoazetidine-1-carboxylate (246 mL, 1.5 mmol), TTMSS (466 mL, 1.5 mmol), NiCl₂-glyme (11 mg, 0.05 mmol), dtbpy (14 mg, 0.05 mmol), Ir[dF (CF3)ppy] (dtbpy) PF6 (11 mg, 0.01 mmol), 2, 6-dimethylpyridine (580 mL, 5 mmol) in DME (5 mL) was stirred under nitrogen at 25° C. and irradiated with a blue LED for 16 hr. The solution was concentrated under reduced pressure. The residue was purified by flash column (DCM/MeOH=40:1) to give the product as light-yellow solid (220 mg, 53%). Mass (m/z): 414.2 [M+H]⁺.

### Step 2

Following general synthesis procedure I, from tert-butyl 3-(2-(2, 6-dioxopiperidin-3-yl)-1, 3-dioxoisoindolin-5-yl) azetidine-1-carboxylate, the product 5-(1-(2-(3-(3-(4-chloro-3-ethyl-1H-pyrrolo [2, 3-b] pyridin-5-yl) phenyl)-2-oxotetrahydropyrimidin-1 (2H)-yl) ethyl) azetidin-3-yl)-2-(2, 6-dioxopiperidin-3-yl) isoindoline-1, 3-dione was obtained (7 mg, yield: 4%) as a white solid. Mass (m/z): 694.1 [M+H]⁺. ¹H NMR (400 MHz, DMSO-d₆) δ 11.77 (d, J=2.4 Hz, 1H), 11.14 (s, 1H), 8.10 (s, 1H), 8.00 (s, 1H), 7.85 (s, 2H), 7.43-7.29 (m, 4H), 7.20 (d, J=7.4 Hz, 1H), 5.14 (dd, J=12.8, 5.4 Hz, 1H), 3.80 (d, J=6.8 Hz, 1H), 3.70 (dt, J=13.8, 5.8 Hz, 4H), 3.44 (t, J=5.8 Hz, 2H), 3.28 (dd, J=15.2, 8.8 Hz, 5H), 2.97-2.80 (m, 3H), 2.59 (dd, J=31.8, 13.6 Hz, 3H), 2.06 (s, 3H), 1.26 (dd, J=13.8, 6.2 Hz, 3H).

### Example 7

### 3-(5-(4-(2-(3-(3-(4-chloro-3-ethyl-1H-pyrrolo [2, 3-b] pyridin-5-yl) phenyl)-2-oxotetrahydropyrimidin-1 (2H)-yl) ethyl) piperazin-1-yl)-6-fluoro-1-oxoisoindolin-2-yl) piperidine-2, 6-dione

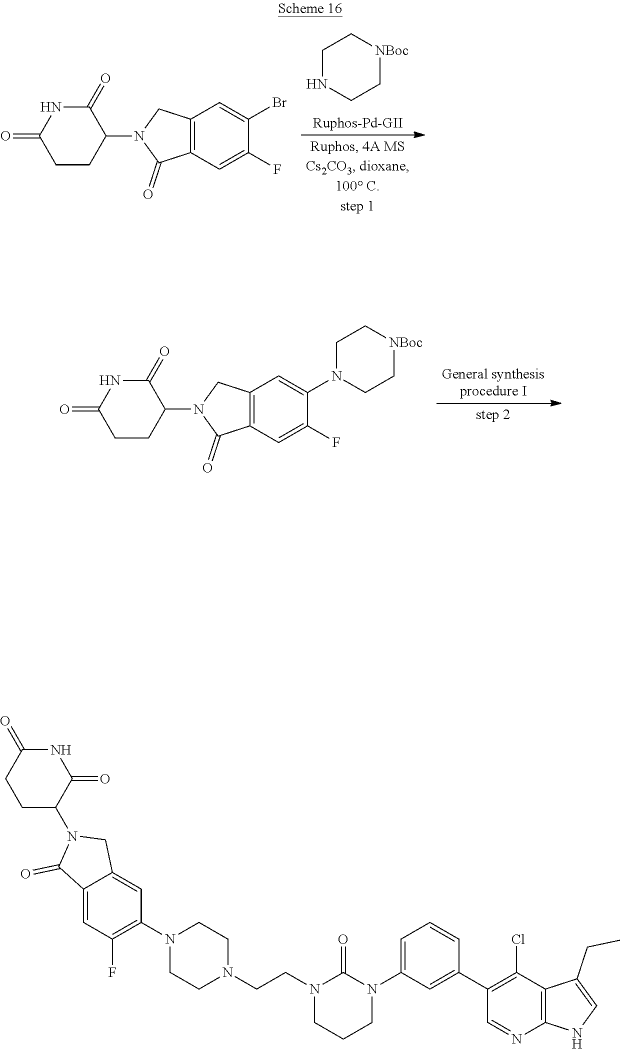 [SMILES: O=C1CCC(N2Cc3cc(Br)c(F)cc3C2=O)C(=O)N1 ; CC(C)(C)OC(=O)N1CCNCC1 ; CC(C)(C)OC(=O)N1CCN(c2cc3c(cc2F)C(=O)N(C2CCC(=O)NC2=O)C3)CC1 ; CCc1c[nH]c2ncc(-c3cccc(N4CCCN(CCN5CCN(c6cc7c(cc6F)C(=O)N(C6CCC(=O)NC6=O)C7)CC5)C4=O)c3)c(Cl)c12]

### Step 1. Preparation of tert-butyl 4-(2-(2, 6-dioxopiperidin-3-yl)-6-fluoro-1-oxoisoindolin-5-yl) piperazine-1-carboxylate

A solution of 3-(5-bromo-6-fluoro-1-oxo-2, 3-dihydro-1H-isoindol-2-yl) piperidine-2, 6-dione (171 mg, 0.5 mmol), tert-butyl piperazine-1-carboxylate (186 mg, 1 mmol), Ruphos-Pd-GII (77 mg, 0.1 mmol), Ruphos (47 mg, 0.1 mmol), Cs₂CO₃ (489 mg, 1.5 mmol), 4A MS (100 mg) in dioxane (10 mL) was stirred under nitrogen at 100° C. for 24 hr. The solution was filtered through celite, concentrated under reduced pressure. The residue was purified by flash column (DCM/MeOH=40:1) to give the product as white solid (70 mg, 31%). Mass (m/z): 447.2 [M+H]⁺.

### Step 2

Following general synthesis procedure I, from tert-butyl 4-(2-(2, 6-dioxopiperidin-3-yl)-6-fluoro-1-oxoisoindolin-5-yl) piperazine-1-carboxylate, the product 3-(5-(4-(2-(3-(3-(4-chloro-3-ethyl-1H-pyrrolo [2, 3-b] pyridin-5-yl) phenyl)-2-oxotetrahydropyrimidin-1 (2H)-yl) ethyl) piperazin-1-yl)-6-fluoro-1-oxoisoindolin-2-yl) piperidine-2, 6-dione was obtained (15 mg, 11%) as a white solid. Mass (m/z): 727.3 [M+H]⁺. ¹H NMR (400 MHz, DMSO-d₆) δ 11.77 (s, 1H), 10.99 (s, 1H), 8.11 (s, 1H), 7.50 (d, J=11.2 Hz, 1H), 7.45 (s, 1H), 7.42 (d, J=7.6 Hz, 1H), 7.40-7.32 (m, 3H), 7.24 (d, J=7.4 Hz, 1H), 5.09 (dd, J=13.2, 4.8 Hz, 1H), 4.36 (d, J=17.2 Hz, 2H), 4.25 (d, J=17.4 Hz, 2H), 3.77 (d, J=5.8 Hz, 3H), 3.71 (s, 2H), 3.45 (dd, J=14.8, 9.4 Hz, 4H), 3.30 (d, J=10.8 Hz, 2H), 3.16 (d, J=11.8 Hz, 2H), 2.96-2.82 (m, 3H), 2.67-2.55 (m, 2H), 2.40-2.35 (m, 1H), 2.10 (s, 2H), 2.03-1.94 (m, 1H), 1.23 (t, J=7.4 Hz, 3H).

### Example 8

### 5-(4-((3-(3-(4-chloro-3-ethyl-1H-pyrrolo [2, 3-b] pyridin-5-yl) phenyl)-2-oxotetrahydropyrimidin-1 (2H)-yl) methyl) piperidin-1-yl)-2-(2, 6-dioxopiperidin-3-yl) isoindoline-1, 3-dione

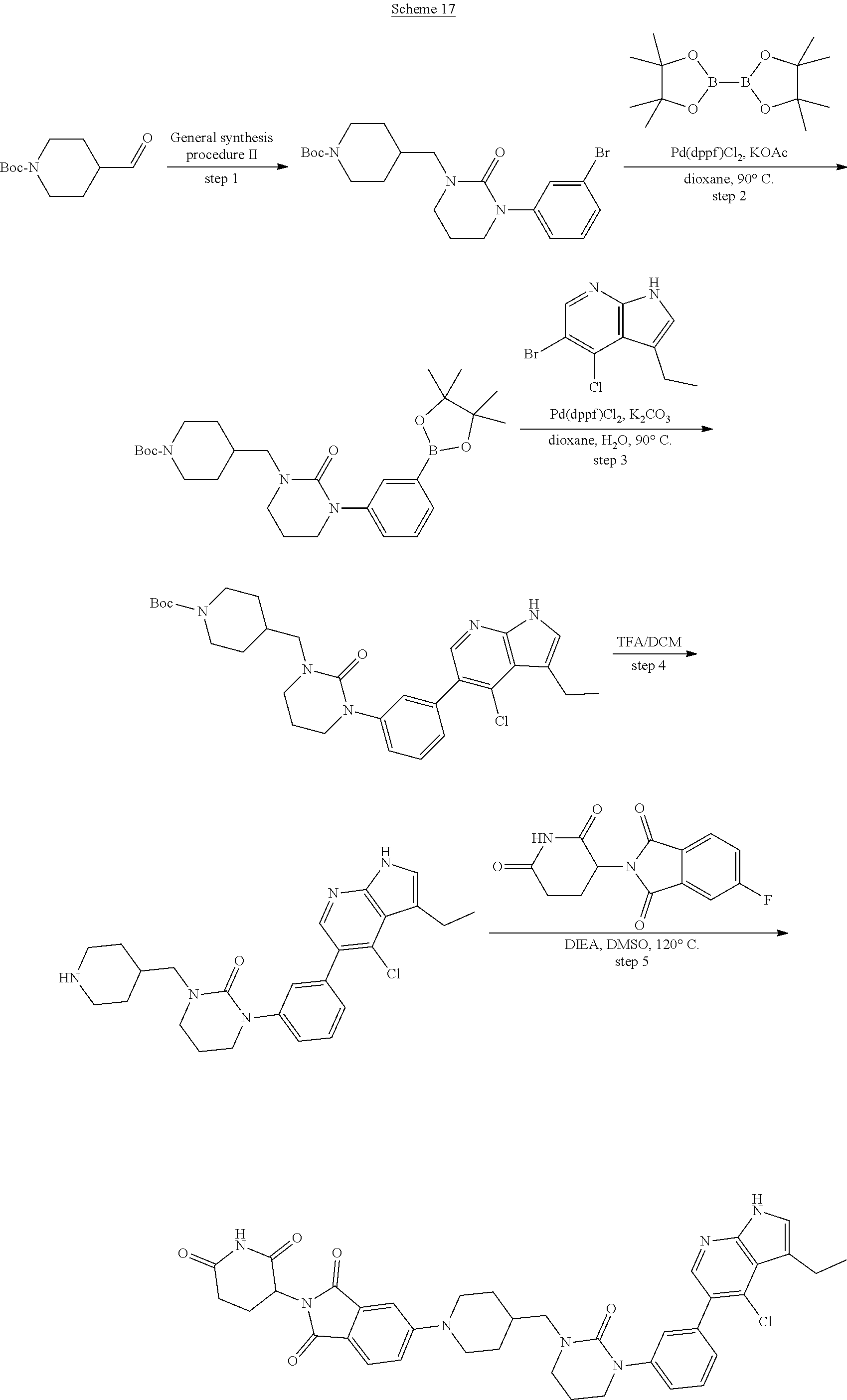

### Step 1

Following general synthesis procedure II, from tert-butyl 4-formylpiperidine-1-carboxylate, product tert-butyl 4-((3-(3-bromophenyl)-2-oxotetrahydropyrimidin-1 (2H)-yl) methyl) piperidine-1-carboxylate was obtained as yellow oil (11.1 g, 58%). Mass (m/z): 473.9 [M+H]⁺.

### Step 2. Preparation of tert-butyl 4-((2-oxo-3-(3-(4, 4, 5, 5-tetramethyl-1, 3, 2-dioxaborolan-2-yl) phenyl) tetrahydropyrimidin-1 (2H)-yl) methyl) piperidine-1-carboxylate

To a solution of tert-butyl 4-((3-(3-bromophenyl)-2-oxotetrahydropyrimidin-1 (2H)-yl) methyl) piperidine-1-carboxylate (11.1 g, 24.48 mmol) in dioxane (200 mL) was added 4, 4, 5, 5-tetramethyl-2-(4, 4, 5, 5-tetramethyl-1, 3, 2-dioxaborolan-2-yl)-1, 3, 2-dioxaborolane (9.3 g, 36.72 mmol), KOAc (7.2 g, 73.45 mmol) and Pd(dppf)Cl₂ (1.79 g, 2.45 mmol). The reaction mixture was stirred at 90° C. under N₂ for 2 hrs. The solvent was removed under reduced pressure and the residue was purified by Combiflash (DCM/MeOH=0˜10%) to give the product as brown oil (12.3 g, 80%). Mass (m/z): 522.0 [M+H]⁺.

### Step 3. Preparation of tert-butyl 4-((3-(3-(4-chloro-3-ethyl-1H-pyrrolo [2, 3-b]pyridin-5-yl) phenyl)-2-oxotetrahydropyrimidin-1 (2H)-yl) methyl) piperidine-1-carboxylate

To a solution of tert-butyl 4-((2-oxo-3-(3-(4, 4, 5, 5-tetramethyl-1, 3, 2-dioxaborolan-2-yl) phenyl) tetrahydropyrimidin-1 (2H)-yl) methyl) piperidine-1-carboxylate (12.3 g, 24.58 mmol) in dioxane/H₂O (10:1, 200 mL) was added 5-bromo-4-chloro-3-ethyl-1H-pyrrolo [2, 3-b] pyridine (6.37 g, 24.58 mmol), K₂CO₃ (10.19 g, 73.73 mmol) and Pd(dppf)Cl₂ (1.79 g, 2.46 mmol). The reaction mixture was stirred at 90° C. under N₂ for 4 hrs. After the reaction completed, H₂O (300 mL) was added to the reaction mixture, and then extracted with DCM (200 mL×3). The combined organic layer was washed with brine (300 mL×2), then dried over anhydrous Na₂SO₄. After filtration, the solution was concentration under vacuum, and the residue was purified by Combiflash (DCM/MeOH=0˜10%) to give the product as a brown solid (5.83 g, 39%). Mass (m/z): 552.0 [M+H]⁺.

### Step 4. Preparation of 1-(3-(4-chloro-3-ethyl-1H-pyrrolo [2, 3-b] pyridin-5-yl) phenyl)-3-(piperidin-4-ylmethyl) tetrahydropyrimidin-2 (1H)-one

To a solution of tert-butyl 4-((3-(3-(4-chloro-3-ethyl-1H-pyrrolo [2, 3-b]pyridin-5-yl) phenyl)-2-oxotetrahydropyrimidin-1 (2H)-yl) methyl) piperidine-1-carboxylate (5.83 g, 12.9 mmol) in DCM/TFA (3:1, 50 mL). The reaction mixture was stirred at rt for 1 hour. The resulting mixture was concentrated to give the product as brown oil (9.58 g, purity: 60%). Mass (m/z): 451.9 [M+H]⁺.

### Step 5. Preparation of 5-(4-((3-(3-(4-chloro-3-ethyl-1H-pyrrolo [2, 3-b] pyridin-5-yl) phenyl)-2-oxotetrahydropyrimidin-1 (2H)-yl) methyl) piperidin-1-yl)-2-(2, 6-dioxopiperidin-3-yl) isoindoline-1, 3-dione

To a solution of 1-(3-(4-chloro-3-ethyl-1H-pyrrolo [2, 3-b] pyridin-5-yl) phenyl)-3-(piperidin-4-ylmethyl) tetrahydropyrimidin-2 (1H)-one (4.23 g, 9.36 mmol) in DMSO (100 mL) was added 2-(2, 6-dioxopiperidin-3-yl)-5-fluoroisoindoline-1, 3-dione (2.585 g, 9.36 mmol) and DIEA (3.63 g, 20.08 mmol). The reaction mixture was stirred at 120° C. under N₂ for 2 hrs. After the reaction completed, H₂O (300 mL) was added to the reaction mixture, and then extracted with EA (200 mL×3). The combined organic layer was washed with brine (300 mL×2), then dried over anhydrous Na₂SO₄. After filtration, the solution was concentration under vacuum, and the residue was purified by Combiflash (DCM/MeOH=0˜10%) to give the product as a yellow solid (1.45 g, 21%). Mass (m/z): 707.7 [M+H]⁺. ¹H NMR (400 MHz, DMSO-d₆) δ 11.79 (s, 1H), 11.08 (s, 1H), 8.11 (s, 1H), 7.64 (d, J=8.6 Hz, 1H), 7.37 (ddd, J=25.2, 13.8, 8.2 Hz, 5H), 7.22 (t, J=9.8 Hz, 2H), 5.06 (dd, J=12.8, 5.4 Hz, 1H), 4.07 (d, J=12.8 Hz, 2H), 3.77-3.70 (m, 2H), 3.39 (t, J=5.8 Hz, 2H), 3.20 (d, J=7.2 Hz, 2H), 3.02-2.82 (m, 5H), 2.62-2.53 (m, 2H), 2.03 (dd, J=12.8, 7.2 Hz, 4H), 1.71 (d, J=11.8 Hz, 2H), 1.28 (t, J=7.4 Hz, 3H).

### Example 9

### 5-(4-(2-(3-(3-(4-chloro-3-cyclopropyl-1H-pyrrolo [2, 3-b] pyridin-5-yl) phenyl)-2-oxotetrahydropyrimidin-1 (2H)-yl) ethyl) piperidin-1-yl)-2-(2, 6-dioxopiperidin-3-yl)-6-fluoroisoindoline-1, 3-dione

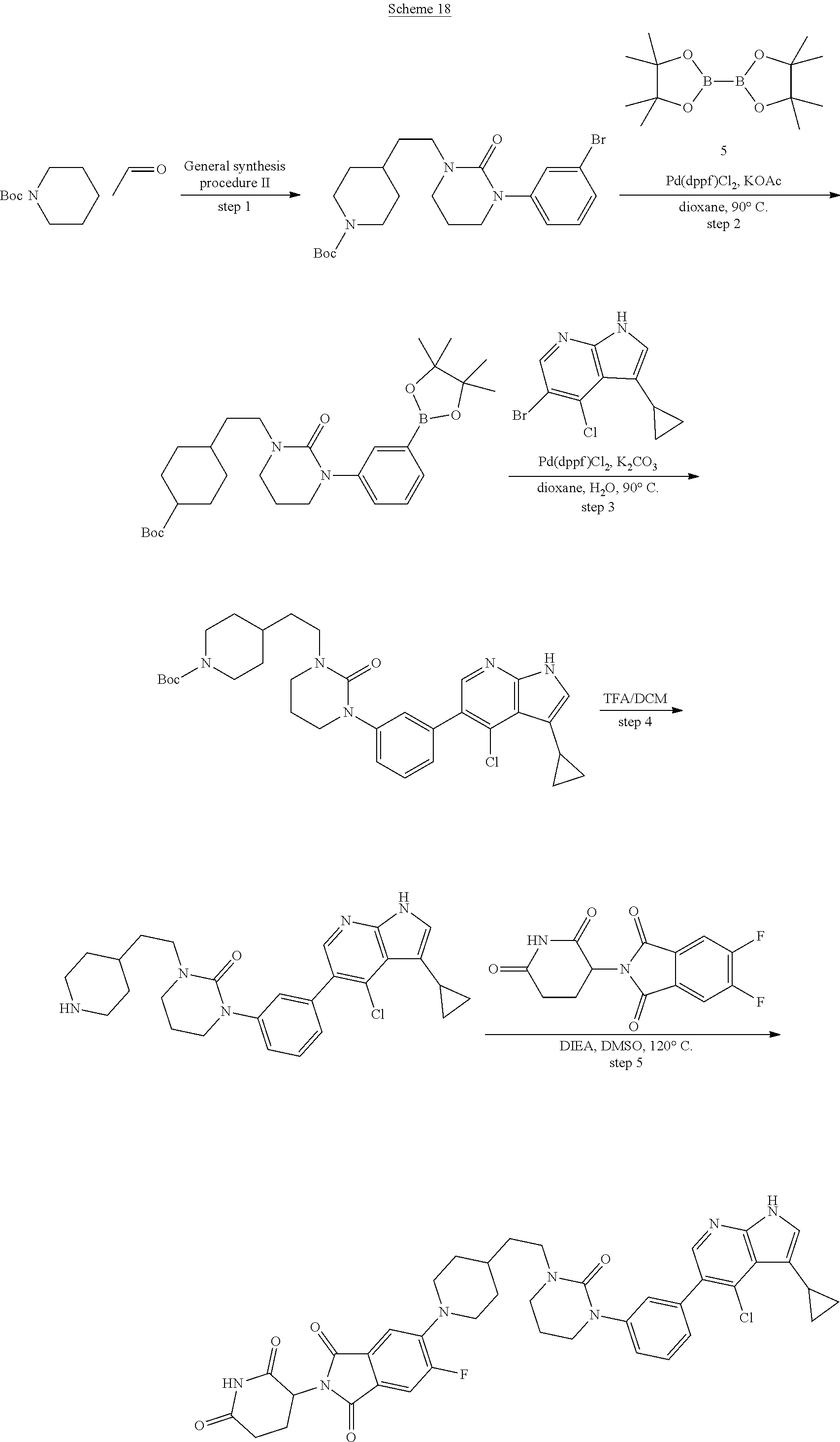

### Step 1

Following general synthesis procedure II, from tert-butyl 4-(2-oxoethyl) piperidine-1-carboxylate, product tert-butyl 4-(2-(3-(3-bromophenyl)-2-oxotetrahydropyrimidin-1 (2H)-yl) ethyl) piperidine-1-carboxylate was obtained (660 mg, 56%) as yellow oil. Mass (m/z): 488.2 [M+H]⁺.

### Step 2. Preparation of tert-butyl 4-(2-(2-oxo-3-(3-(4, 4, 5, 5-tetramethyl-1, 3, 2-dioxa borolan-2-yl) phenyl) tetrahydropyrimidin-1 (2H)-yl) ethyl) piperidine-1-carboxylate

To a solution of tert-butyl 4-(2-(3-(3-bromophenyl)-2-oxotetrahydropyrimidin-1 (2H)-yl) ethyl) piperidine-1-carboxylate (660 mg, 1.41 mmol) in dioxane (20 mL) was added 4, 4, 4′, 4′, 5, 5, 5′, 5′-octamethyl-2, 2′-bi (1, 3, 2-dioxaborolane) (538 mg, 2.12 mmol), KOAc (416 mg, 4.24 mmol) and Pd(dppf)Cl₂ (103 mg, 0.14 mmol). The reaction mixture was stirred at 90° C. under N₂ for 2 hrs. The solvent was removed under reduced pressure and the residue was purified by Combiflash (DCM/MeOH=0˜10%) to give the product (650 mg, 89%) as brown oil. Mass (m/z): 536.0 [M+H]⁺.

### Step 3. Preparation of tert-butyl 4-(2-(3-(3-(4-chloro-3-cyclopropyl-1H-pyrrolo [2, 3-b] pyridin-5-yl) phenyl)-2-oxotetrahydropyrimidin-1 (2H)-yl) ethyl) piperidine-1-carboxylate

To a solution of tert-butyl 4-(2-(2-oxo-3-(3-(4, 4, 5, 5-tetramethyl-1, 3, 2-dioxaborolan-2-yl) phenyl) tetrahydropyrimidin-1 (2H)-yl) ethyl) piperidine-1-carboxylate (650 mg, 1.26 mmol) in dioxane/H₂O (10:1, 15 mL) was added 5-bromo-4-chloro-3-cyclopropyl-1H-pyrrolo [2, 3-b] pyridine (343 mg, 1.26 mmol), K₂CO₃ (523 mg, 3.79 mmol) and Pd(dppf)Cl₂ (92 mg, 0.13 mmol). The reaction mixture was stirred at 90° C. under N₂ for 4 hrs. After the reaction completed, H₂O (20 mL) was added to the reaction mixture, and then extracted with DCM (20 mL×3). The combined organic layer was washed with brine (30 mL×2), then dried over anhydrous Na₂SO₄. After filtration, the solution was concentration under vacuum, and the residue was purified by Combiflash (DCM/MeOH=0˜10%) to give the product (250 mg, 34%) as yellow oil. Mass (m/z): 578.3 [M+H]⁺.

### Step 4. Preparation of 1-(3-(4-chloro-3-cyclopropyl-1H-pyrrolo [2, 3-b] pyridin-5-yl) phenyl)-3-(2-(piperidin-4-yl) ethyl) tetrahydropyrimidin-2 (1H)-one

To a solution of tert-butyl 4-(2-(3-(3-(4-chloro-3-cyclopropyl-1H-pyrrolo [2, 3-b] pyridin-5-yl) phenyl)-2-oxotetrahydropyrimidin-1 (2H)-yl) ethyl) piperidine-1-carboxylate (250 mg, 0.43 mmol) in TFA/DCM (3:1, 10 mL). The reaction mixture was stirred at rt for 1 hour. The resulting mixture was concentrated to give the product (230 mg, purity: 89%) as brown oil. Mass (m/z): 477.9 [M+H]⁺.

### Step 5. Preparation of 5-(4-(2-(3-(3-(4-chloro-3-cyclopropyl-1H-pyrrolo [2, 3-b]pyridin-5-yl) phenyl)-2-oxotetrahydropyrimidin-1 (2H)-yl) ethyl) piperidin-1-yl)-2-(2, 6-dioxopiperidin-3-yl)-6-fluoroisoindoline-1, 3-dione

To a solution of 1-(3-(4-chloro-3-cyclopropyl-1H-pyrrolo [2, 3-b] pyridin-5-yl) phenyl)-3-(2-(piperidin-4-yl) ethyl) tetrahydropyrimidin-2 (1H)-one (230 mg, 0.48 mmol) in DMSO (10 mL) was added 2-(2, 6-dioxopiperidin-3-yl)-5, 6-difluoro isoindoline-1, 3-dione (142 mg, 0.48 mmol) and DIEA (187 mg, 1.44 mmol). The reaction mixture was stirred at 120° C. under N₂ for 2 hrs. After the reaction completed, H₂O (20 mL) was added to the reaction mixture, and then extracted with EA (20 mL×3). The combined organic layer was washed with brine (30 mL×3), then dried over anhydrous Na₂SO₄. After filtration, the solution was concentration under vacuum, and the residue was purified by Combiflash (DCM/MeOH=0˜10%) to give the product as a yellow solid (85 mg, 23%). Mass (m/z): 751.7 [M+H]⁺. ¹H NMR (400 MHz, DMSO-d₆) δ 11.73 (d, J=2.4 Hz, 1H), 11.11 (s, 1H), 8.11 (s, 1H), 7.69 (d, J=11.6 Hz, 1H), 7.44-7.36 (m, 3H), 7.34-7.28 (m, 2H), 7.23-7.19 (m, 1H), 5.10 (dd, J=12.8, 5.4 Hz, 1H), 3.75-3.69 (m, 2H), 3.59 (d, J=12.4 Hz, 2H), 3.36 (d, J=10.2 Hz, 4H), 2.90-2.82 (m, 3H), 2.56 (dd, J=19.8, 10.6 Hz, 2H), 2.23-2.15 (m, 1H), 2.04 (dd, J=14.6, 9.2 Hz, 3H), 1.84 (d, J=11.8 Hz, 2H), 1.50 (d, J=6.8 Hz, 3H), 1.37-1.28 (m, 2H), 0.83 (ddd, J=8.2, 6.0, 4.0 Hz, 2H), 0.66-0.60 (m, 2H).

### Example 10

### 5-(4-((3-(3-(4-chloro-3-ethyl-1H-pyrrolo [2, 3-b] pyridin-5-yl) phenyl)-2-oxotetrahydropyrimidin-1 (2H)-yl) methyl) piperidin-1-yl)-2-(1-methyl-2, 6-dioxopiperidin-3-yl) isoindoline-1, 3-dione

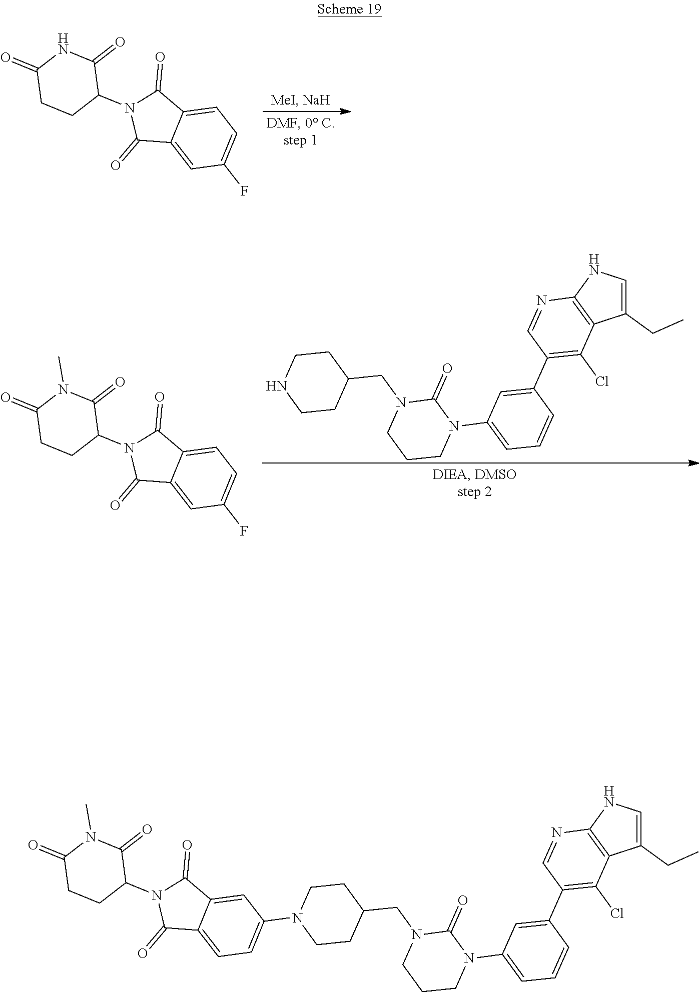 [SMILES: O=C1CCC(N2C(=O)c3ccc(F)cc3C2=O)C(=O)N1 ; CN1C(=O)CCC(N2C(=O)c3ccc(F)cc3C2=O)C1=O ; CCc1c[nH]c2ncc(-c3cccc(N4CCCN(CC5CCNCC5)C4=O)c3)c(Cl)c12 ; CCc1c[nH]c2ncc(-c3cccc(N4CCCN(CC5CCN(c6ccc7c(c6)C(=O)N(C6CCC(=O)N(C)C6=O)C7=O)CC5)C4=O)c3)c(Cl)c12]

### Step 1. Preparation of 5-fluoro-2-(1-methyl-2, 6-dioxopiperidin-3-yl) isoindoline-1, 3-dione

To a solution of 2-(2, 6-dioxopiperidin-3-yl)-5-fluoroisoindoline-1, 3-dione (1.3 g, 4.7 mmol) in DMF (20 mL) was added NaH (60% in oil, 226 mg, 9.41 mmol) at 0° C. The mixture was stirred at 0° C. under N₂ for 0.5 hr. Then CH₃I (1002 mg, 7.06 mmol) was added. The reaction mixture was stirred at rt under N₂ for 2.5 hrs. The reaction mixture was poured into ice water (50 mL) and then extracted with EA (50 mL×3). The combined organic layer was washed with brine (100 mL×3), dried over anhydrous Na₂SO₄. After filtration, the solution was concentration under vacuum, and the residue was purified by Combiflash column (DCM/MeOH=0˜10%) to give the product (375 mg, 27%) as a yellow solid. Mass (m/z): 291.0 [M+H]⁺.

### Step 2. Preparation of 5-(4-((3-(3-(4-chloro-3-ethyl-1H-pyrrolo [2, 3-b] pyridin-5-yl) phenyl)-2-oxotetrahydropyrimidin-1 (2H)-yl) methyl) piperidin-1-yl)-2-(1-methyl-2, 6-dioxopiperidin-3-yl) isoindoline-1, 3-dione

To a solution 1-(3-(4-chloro-3-ethyl-1H-pyrrolo [2, 3-b] pyridin-5-yl) phenyl)-3-(piperidin-4-ylmethyl) tetrahydropyrimidin-2 (1H)-one (400 mg, 0.89 mmol) in DMSO (5 mL) was added 5-fluoro-2-(1-methyl-2, 6-dioxopiperidin-3-yl) isoindoline-1, 3-dione (257 mg, 0.89 mmol) and DIEA (343 mg, 2.66 mmol). The reaction mixture was stirred at 120° C. under N₂ for 2 hrs. After the reaction completed, the reaction mixture was poured into water (15 mL) and then extracted with EA (15 mL×3). The combined organic layer was washed with brine (20 mL×3), then dried over anhydrous Na₂SO₄. Then by filtration, the filtrate was concentrated and the residue was purified by Prep-HPLC [Gemini-C18, 150×21.2 mm, 5 um; ACN-H₂O (0.1% FA) 30-50] to give the product 5-(4-((3-(3-(4-chloro-3-ethyl-1H-pyrrolo [2, 3-b] pyridin-5-yl) phenyl)-2-oxotetrahydropyrimidin-1 (2H)-yl) methyl) piperidin-1-yl)-2-(1-methyl-2, 6-dioxo piperidin-3-yl) isoindoline-1, 3-dione as a yellow solid (59 mg, 9%). Mass (m/z): 722.2 [M+H]⁺. ¹H NMR (400 MHz, DMSO-d₆) δ 11.79 (d, J=2.4 Hz, 1H), 8.11 (s, 1H), 7.64 (d, J=8.4 Hz, 1H), 7.36 (ddd, J=19.0, 7.8, 2.3 Hz, 5H), 7.26-7.18 (m, 2H), 5.13 (dd, J=13.0, 5.4 Hz, 1H), 4.07 (d, J=13.2 Hz, 2H), 3.77-3.70 (m, 2H), 3.39 (t, J=5.8 Hz, 2H), 3.20 (d, J=7.2 Hz, 2H), 3.01 (s, 3H), 2.99-2.87 (m, 5H), 2.55 (d, J=5.0 Hz, 1H), 2.05 (d, J=5.2 Hz, 4H), 1.70 (s, 2H), 1.28 (t, J=7.4 Hz, 3H).

### Example 11

### 3-[6-(4-{[3-(3-{4-chloro-3-cyclopropyl-1H-pyrrolo [2, 3-b] pyridin-3-yl}phenyl)-2-oxo-1, 3-diazinan-1-yl] methyl} piperidin-1-yl)-1-oxo-3H-isoindol-2-yl]piperidine-2, 6-dione

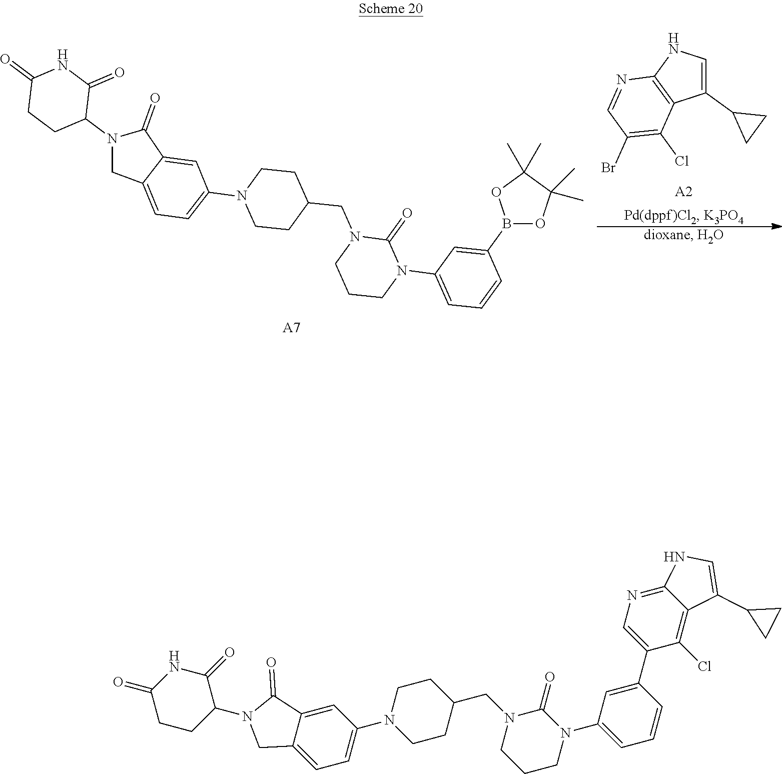

To a solution of 33-{1-oxo-6-[4-({2-oxo-3-[3-(4, 4, 5, 5-tetramethyl-1, 3, 2-dioxaborolan-2-yl) phenyl]-1, 3-diazinan-1-yl} methyl) piperidin-1-yl]-3H-isoindol-2-yl} piperidine-2, 6-dione (100 mg, 0.15 mmol), 3-bromo-4-chloro-3-cyclopropyl-1H-pyrrolo [2, 3-b] pyridine (50.8 mg, 0.18 mmol) and K₃PO₄ (99 mg, 0.46 mmol) in 1.4-dioxane/H₂O (10:1, 3 mL) was added Pd(dppf)Cl₂ (11 mg, 0.015 mmol). The solution was stirred under nitrogen at 85° C. for 16 hrs. The reaction was cooled to room temperature. Water (15 mL) was added and the mixture was extracted with EA (10 mL×3). The combined organic layers were washed with brine (20 mL×2), dried over Na₂SO₄. Then by filtration, the filtrate was concentrated. The residue was purified by Prep-HPLC [Gemini-C18, 150×21.2 mm, Sum; ACN-H₂O (0.1% TFA), 30-50] to give the product as a yellow solid (2.3 mg, 2%). Mass (m/z): 706.3 [M+H]⁺. ¹H NMR (400 MHz, DMSO-d₆) δ 11.75 (s, 1H), 10.97 (s, 1H), 8.11 (s, 1H), 7.54 (s, 2H), 7.45-7.36 (m, 2H), 7.31 (dd, J=22.0, 12.8 Hz, 2H), 7.25-7.19 (m, 1H), 7.15 (s, 1H), 5.20-4.96 (m, 1H), 4.32 (d, J=16.7 Hz, 1H), 4.19 (d, J=16.6 Hz, 1H), 3.75 (dd, J=14.0, 9.0 Hz, 4H), 3.40 (s, 2H), 3.22 (d, J=7.2 Hz, 2H), 3.00-2.88 (m, 2H), 2.77-2.64 (m, 2H), 2.24 (dd, J=28.6, 23.6 Hz, 2H), 2.12-2.00 (m, 2H), 1.91 (d, J=39.8 Hz, 2H), 1.72 (d, J=11.2 Hz, 2H), 1.32-1.21 (m, 2H), 0.90-0.79 (m, 2H), 0.73-0.59 (m, 2H).

### Example 12

### 3-[5-(4-{[3-(3-{4-chloro-3-cyclopropyl-1H-pyrrolo [2, 3-b] pyridin-3-yl}phenyl)-2-oxo-1, 3-diazinan-1-yl] methyl} piperidin-1-yl)-1-oxo-3H-isoindol-2-yl]piperidine-2, 6-dione

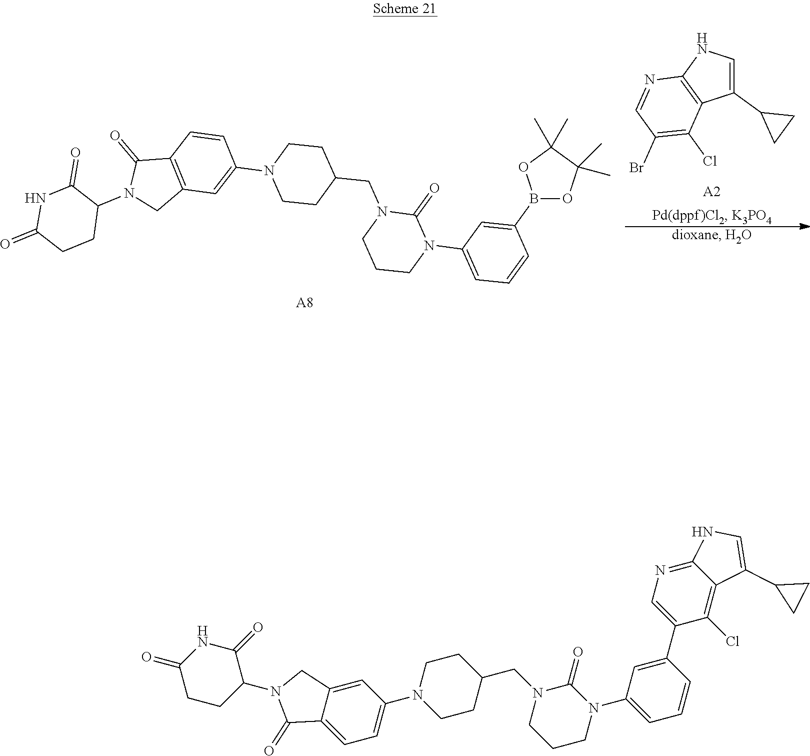

To a solution of 3-{1-oxo-5-[4-({2-oxo-3-[3-(4, 4, 5, 5-tetramethyl-1, 3, 2-dioxaborolan-2-yl) phenyl]-1, 3-diazinan-1-yl} methyl) piperidin-1-yl]-3H-isoindol-2-yl} piperidine-2, 6-dione (70 mg, 0.11 mmol), 3-bromo-4-chloro-3-cyclopropyl-1H-pyrrolo [2, 3-b] pyridine (30 mg, 0.11 mmol) and K₃PO₄ (69 mg, 0.33 mmol) in 1.4-dioxane/H₂O (10:1, 3 mL) was added Pd(dppf)Cl₂ (8 mg, 0.011 mmol). The solution was stirred under nitrogen at 85° C. for 16 hrs. The reaction was cooled to room temperature. Water (15 mL) was added, and the mixture was extracted with EA (10 mL×3). The combined organic layers were washed with brine (20 mL×2), dried over Na₂SO₄. Then by filtration, the filtrate was concentrated. The residue was purified by Prep-HPLC [Gemini-C18, 150×21.2 mm, 5 um; ACN-H₂O (0.1% TFA), 35-50] to give the product as a yellow solid (1.3 mg, 1.5%). Mass (m/z): 706.3 [M+H]. ¹H NMR (400 MHz, DMSO-d₆) δ 11.74 (s, 1H), 10.94 (s, 1H), 8.10 (s, 1H), 7.56 (s, 1H), 7.49 (d, J=8.6 Hz, 1H), 7.37 (s, 2H), 7.35-7.28 (m, 2H), 7.21 (d, J=7.4 Hz, 1H), 7.05 (s, 1H), 5.03 (dd, J=13.4, 5.2 Hz, 1H), 4.31 (d, J=16.8 Hz, 2H), 4.18 (d, J=16.6 Hz, 2H), 3.89 (d, J=11.6 Hz, 2H), 3.74 (s, 2H), 3.21 (d, J=6.8 Hz, 2H), 2.85 (d, J=10.6 Hz, 2H), 2.20 (d, J=4.6 Hz, 2H), 2.06 (s, 2H), 1.94 (s, 2H), 1.70 (d, J=10.8 Hz, 2H), 1.23 (s, 4H), 0.88-0.80 (m, 2H), 0.64 (d, J=3.4 Hz, 2H).

### Example 13

### 3-(6-{4-[(3-{3-[4-chloro-3-(2, 2-difluoroethyl)-1H-pyrrolo [2, 3-b] pyridin-3-yl]phenyl}-2-oxo-1, 3-diazinan-1-yl) methyl] piperidin-1-yl}-1-oxo-3H-isoindol-2-yl) piperidine-2, 6-dione

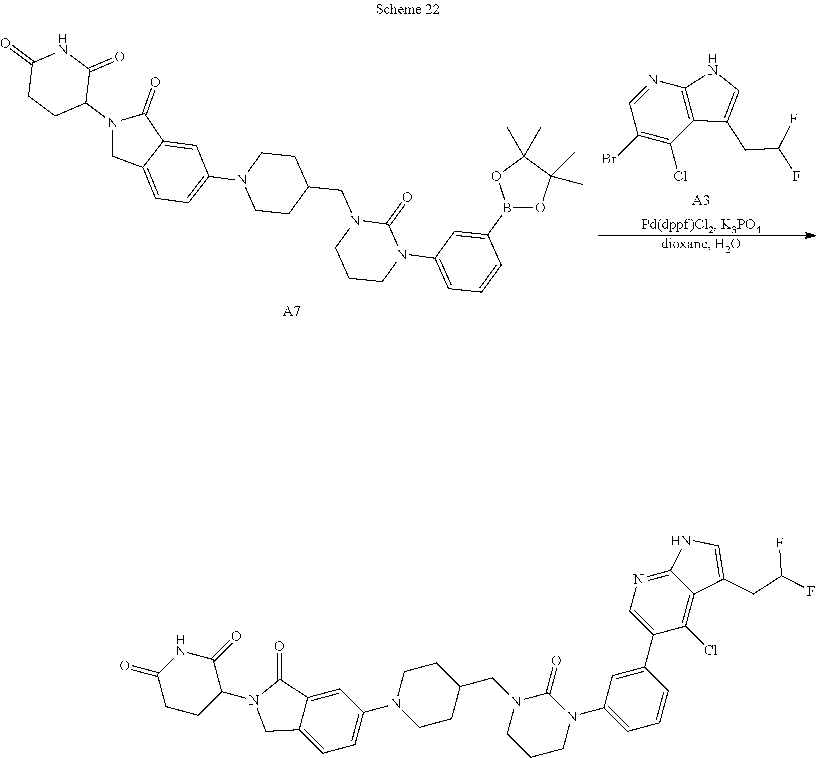

To a solution of 3-{1-oxo-6-[4-({2-oxo-3-[3-(4, 4, 5, 5-tetramethyl-1, 3, 2-dioxaborolan-2-yl) phenyl]-1, 3-diazinan-1-yl} methyl) piperidin-1-yl]-3H-isoindol-2-yl} piperidine-2, 6-dione (100 mg, 0.15 mmol), 3-bromo-4-chloro-3-(2, 2-difluoroethyl)-1H-pyrrolo [2, 3-b] pyridine (55 mg, 0.18 mmol and K₃PO₄ (99 mg, 0.46 mmol) in 1.4-dioxane/H₂O (5 mL) was added Pd(dppf)Cl₂ (11 mg, 0.015 mmol). The solution was stirred under nitrogen at 85° C. for 16 hrs. The reaction was cooled to room temperature. Water (15 mL) was added, and the mixture was extracted with EA (10 mL×3). The combined organic layers were washed with brine (20 mL×2), dried over Na₂SO₄. Then by filtration, the filtrate was concentrated. The crude product was purified by Prep-HPLC [Gemini-C18, 150×21.2 mm, 5 um; ACN-H₂O (0.1% FA), 30-50] to give the product as a yellow solid (6 mg, 5.1%). Mass (m/z): 730.3 [M+H]⁺. ¹H NMR (400 MHz, DMSO-d₆) δ 12.10 (d, J=1.8 Hz, 1H), 10.97 (s, 1H), 8.15 (d, J=12.4 Hz, 1H), 7.58 (d, J=2.0 Hz, 1H), 7.38 (td, J=15.2, 7.8 Hz, 4H), 7.23 (dd, J=16.4, 8.0 Hz, 2H), 7.15 (s, 1H), 6.48-6.12 (m, 1H), 5.09 (dd, J=13.2, 5.0 Hz, 1H), 4.32 (d, J=16.8 Hz, 1H), 4.19 (d, J=16.8 Hz, 1H), 3.86-3.66 (m, 4H), 3.52 (td, J=17.2, 4.2 Hz, 2H), 3.44-3.37 (m, 2H), 3.22 (d, J=7.2 Hz, 2H), 2.96-2.85 (m, 1H), 2.71 (t, J=11.5 Hz, 2H), 2.58 (d, J=17.0 Hz, 1H), 2.42-2.29 (m, 1H), 2.10-2.00 (m, 2H), 1.98-1.85 (m, 2H), 1.71 (d, J=11.2 Hz, 2H), 1.32-1.23 (m, 2H).

### Example 14

### 3-(5-{4-[(3-{3-[4-chloro-3-(2, 2-difluoroethyl)-1H-pyrrolo [2, 3-b] pyridin-3-yl]phenyl}-2-oxo-1, 3-diazinan-1-yl) methyl] piperidin-1-yl}-1-oxo-3H-isoindol-2-yl) piperidine-2, 6-dione

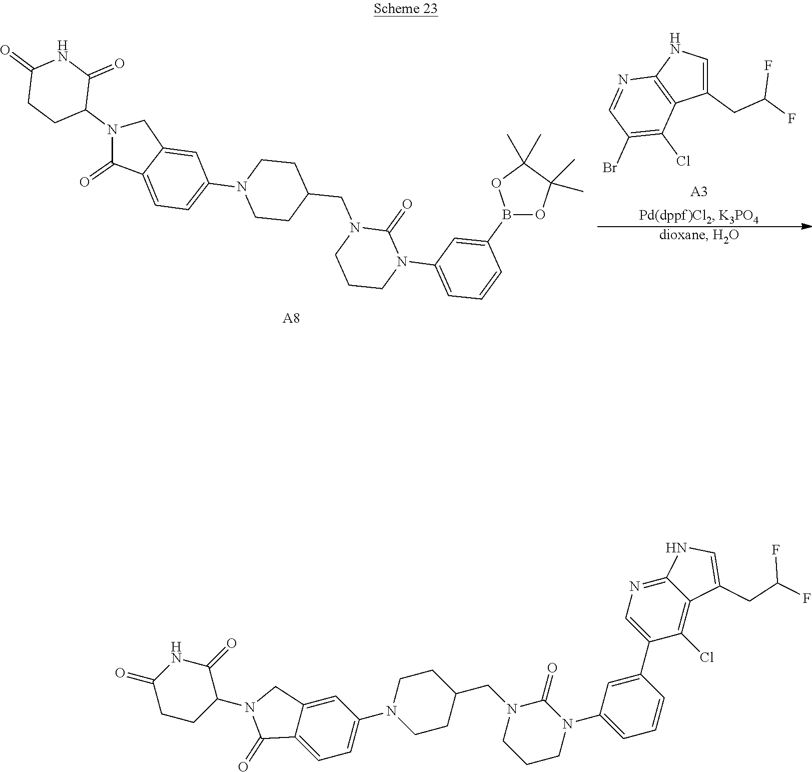

To a solution of 3-{1-oxo-5-[4-({2-oxo-3-[3-(4, 4, 5, 5-tetramethyl-1, 3, 2-dioxaborolan-2-yl) phenyl]-1, 3-diazinan-1-yl} methyl) piperidin-1-yl]-3H-isoindol-2-yl} piperidine-2, 6-dione (70 mg, 0.11 mmol), 5-bromo-4-chloro-3-(2, 2-difluoroethyl)-1H-pyrrolo [2, 3-b] pyridine (39 mg, 0.11 mmol) and K₃PO₄ (69 mg, 0.33 mmol) in 1.4-dioxane/H₂O (10:1, 3 mL) was added Pd(dppf)Cl₂ (8 mg, 0.011 mmol). The solution was stirred under nitrogen at 85° C. for 16 hrs. The reaction was cooled to room temperature. Water (15 mL) was added, and the mixture was extracted with EA (10 mL×3). The combined organic layers were washed with brine (20 mL×2), dried over Na₂SO₄. Then by filtration, the filtrate was concentrated. The residue was purified by Prep-HPLC [Gemini-C18, 150×21.2 mm, 5 um; ACN-H₂O (0.1% FA), 35-50] to give the product as a yellow solid (2 mg, 2.5%). Mass (m/z): 730.3 [M+H]⁺. ¹H NMR (400 MHz, DMSO-d₆) δ 12.09 (s, 1H), 10.93 (s, 1H), 8.40 (s, 1H), 8.16 (s, 1H), 7.58 (s, 1H), 7.49 (d, J=8.6 Hz, 1H), 7.43-7.27 (m, 3H), 7.21 (d, J=7.6 Hz, 1H), 7.03 (d, J=7.8 Hz, 1H), 6.30 (s, 1H), 5.03 (d, J=12.6 Hz, 1H), 4.31 (d, J=17.0 Hz, 1H), 4.18 (d, J=17.2 Hz, 1H), 3.89 (d, J=11.4 Hz, 2H), 3.74 (s, 2H), 3.54 (d, J=16.6 Hz, 2H), 3.40 (s, 2H), 3.20 (s, 2H), 2.85 (dd, J=25.6, 13.0 Hz, 2H), 2.63 (d, J=28.0 Hz, 2H), 2.33 (s, 1H), 2.06 (s, 2H), 1.93 (s, 2H), 1.70 (d, J=11.0 Hz, 2H), 1.24 (s, 2H).

### Example 15

### 3-((4-(4-((3-(3-(4-chloro-3-ethyl-1H-pyrrolo [2, 3-b] pyridin-5-yl) phenyl)-2-oxotetrahydropyrimidin-1 (2H)-yl) methyl) piperidin-1-yl)-3-fluorophenyl) amino) piperidine-2, 6-dione

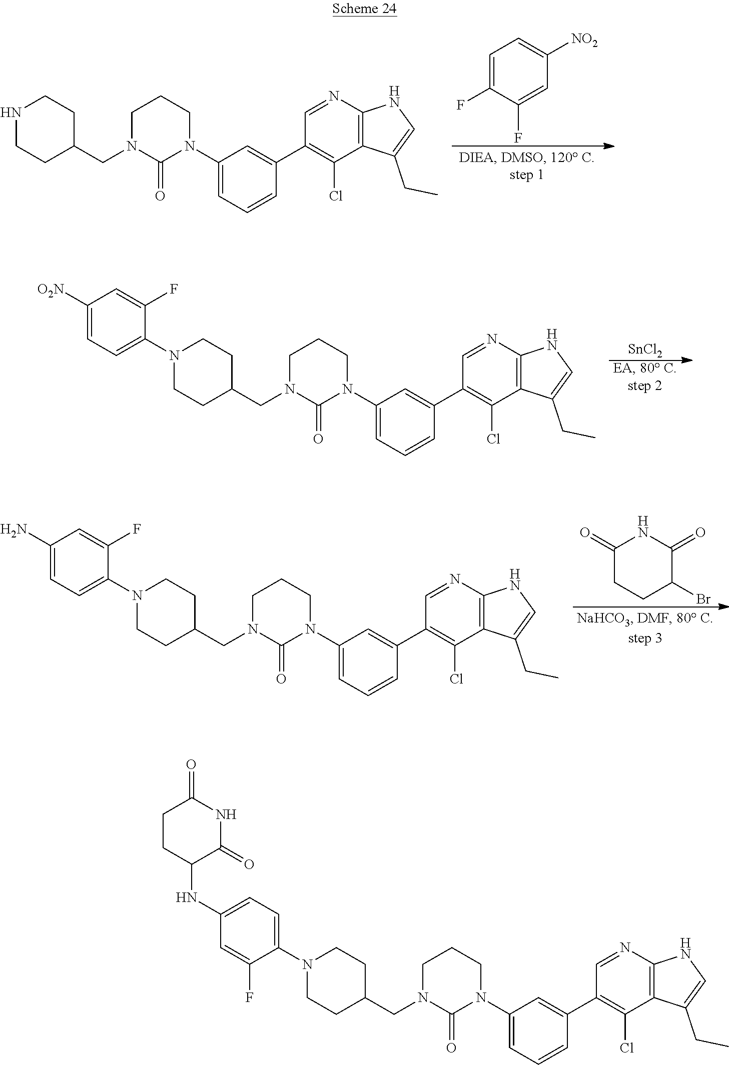

Step 1. Preparation of 1-(3-{4-chloro-3-ethyl-1H-pyrrolo [2, 3-b] pyridin-3-yl}phenyl)-3-{[1-(2-fluoro-4-nitrophenyl) piperidin-4-yl] methyl}-1, 3-diazinan-2-one: To a solution of 1-(3-{4-chloro-3-ethyl-1H-pyrrolo [2, 3-b] pyridin-3-yl} phenyl)-3-(piperidin-4-ylmethyl)-1, 3-diazinan-2-one (700 mg, 1.55 mmol) in DMSO (20 mL) was added 1, 2-difluoro-4-nitrobenzene (246 mg, 1.55 mmol) and DIEA (1001 mg, 7.75 mmol). The reaction mixture was stirred at 120° C. under N₂ for 1 hour. After the reaction completed, H₂O (100 mL) was added to the reaction mixture, and then extracted with EA (50 mL×3). The combined organic layer was washed with brine (50 mL×3), then dried over anhydrous Na₂SO₄. After filtration, the solution was concentrated under vacuum, and the residue was purified by Combiflash (DCM/MeOH=0˜10%) to give the product 1-(3-{4-chloro-3-ethyl-1H-pyrrolo [2, 3-b] pyridin-3-yl} phenyl)-3-{[1-(2-fluoro-4-nitrophenyl) piperidin-4-yl] methyl}-1, 3-diazinan-2-one as a yellow solid (660 mg, 72%). Mass (m/z): 590.9 [M+H]⁺.

Step 2. Preparation of 1-{[1-(4-amino-2-fluorophenyl) piperidin-4-yl] methyl}-3-(3-{4-chloro-3-ethyl-1H-pyrrolo [2, 3-b] pyridin-3-yl} phenyl)-1, 3-diazinan-2-one: A solution of 1-(3-{4-chloro-3-ethyl-1H-pyrrolo [2, 3-b] pyridin-3-yl} phenyl)-3-{[1-(2-fluoro-4-nitrophenyl) piperidin-4-yl] methyl}-1, 3-diazinan-2-one (650 mg, 1.1 mmol) in EA (10 mL) was stirred at 80° C. for 16 hrs. The solvent was removed under reduced pressure and the residue was purified by Combiflash [DCM/MeOH (10% NH₃·H₂O)=0˜10%] to give the product 1-{[1-(4-amino-2-fluorophenyl) piperidin-4-yl] methyl}-3-(3-{4-chloro-3-ethyl-1H-pyrrolo [2, 3-b] pyridin-3-yl} phenyl)-1, 3-diazinan-2-one as a brown solid (450 mg, 72%). Mass (m/z): 561.0 [M+H]⁺.

Step 3. Preparation of 3-((4-(4-((3-(3-(4-chloro-3-ethyl-1H-pyrrolo [2, 3-b]pyridin-5-yl) phenyl)-2-oxotetrahydropyrimidin-1 (2H)-yl) methyl) piperidin-1-yl)-3-fluorophenyl) amino) piperidine-2, 6-dione: To a solution of 1-{[1-(4-amino-2-fluorophenyl) piperidin-4-yl] methyl}-3-(3-{4-chloro-3-ethyl-1H-pyrrolo [2, 3-b]pyridin-3-yl} phenyl)-1, 3-diazinan-2-one (200 mg, 0.36 mmol) in DMF (10 mL) was added 3-bromopiperidine-2, 6-dione (342 mg, 1.8 mmol) and NaHCO₃ (90 mg, 1.07 mmol). The reaction mixture was stirred at 80° C. under N₂ for 16 hrs. The solvent was removed under reduced pressure and the residue was purified by Combiflash (DCM/MeOH=0˜10%) to give the product 3-((4-(4-((3-(3-(4-chloro-3-ethyl-1H-pyrrolo [2, 3-b] pyridin-5-yl) phenyl)-2-oxotetrahydropyrimidin-1 (2H)-yl) methyl) piperidin-1-yl)-3-fluorophenyl) amino) piperidine-2, 6-dione as a white solid (4 mg, 1%). Mass (m/z): 672.0 [M+H]⁺. ¹H NMR (400 MHz, MeOD) δ 8.11 (s, 1H), 7.47 (t, J=7.8 Hz, 1H), 7.38 (s, 1H), 7.33 (t, J=7.2 Hz, 2H), 7.26 (s, 1H), 6.99 (s, 1H), 6.54 (d, J=17.8 Hz, 2H), 4.24 (s, 1H), 3.84-3.75 (m, 2H), 3.52 (t, J=5.8 Hz, 2H), 3.34 (d, J=7.2 Hz, 4H), 3.01 (q, J=7.4 Hz, 2H), 2.86-2.68 (m, 4H), 2.28 (s, 1H), 2.18 (dd, J=13.4, 7.4 Hz, 2H), 1.88 (d, J=20.2 Hz, 4H), 1.53 (s, 2H), 1.33 (t, J=7.4 Hz, 4H).

### Example 16

### 3-((4-(4-(2-(3-(3-(4-chloro-3-ethyl-1H-pyrrolo [2, 3-b] pyridin-5-yl) phenyl)-2-oxotetrahydropyrimidin-1 (2H)-yl) ethyl) piperidin-1-yl)-3-fluorophenyl) amino) piperidine-2, 6-dione

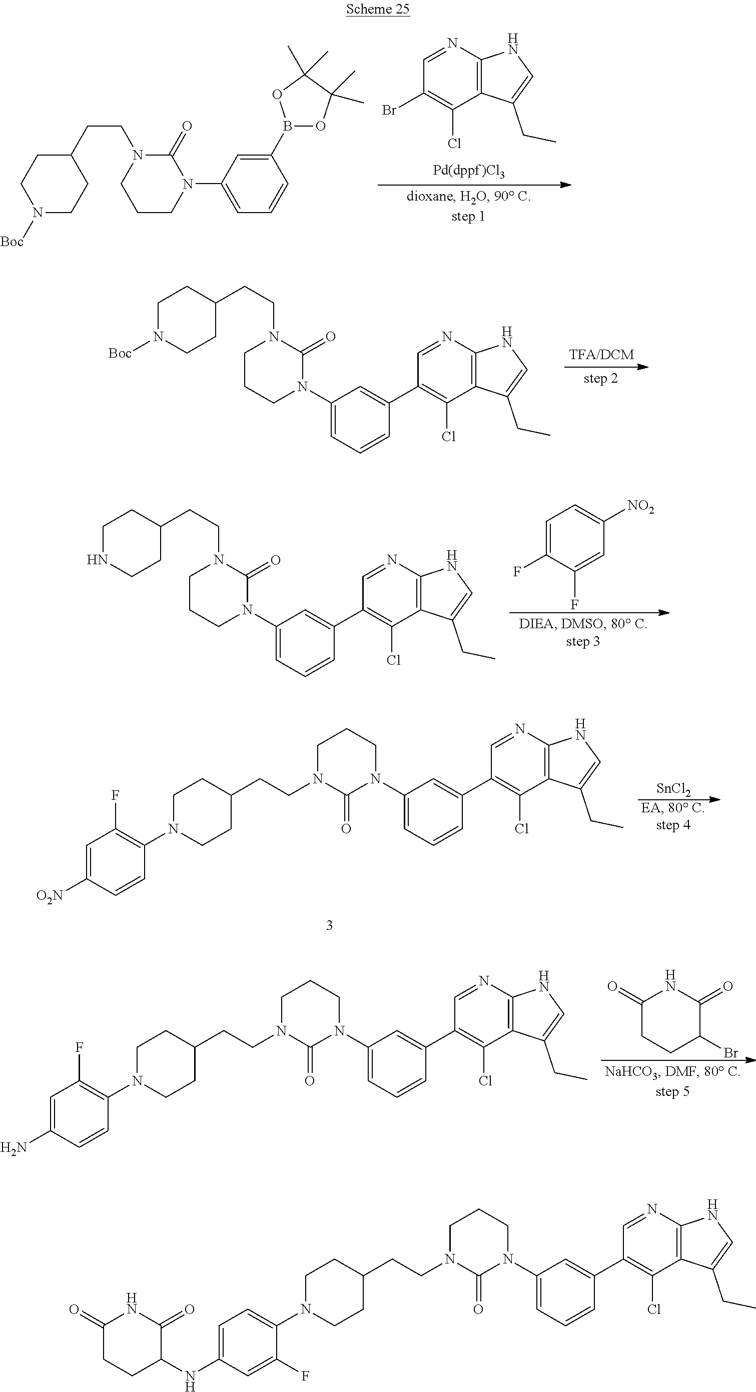

Step 1. Preparation of tert-butyl 4-(2-(3-(3-(4-chloro-3-ethyl-1H-pyrrolo[2, 3-b] pyridin-5-yl)phenyl)-2-oxotetrahydropyrimidin-1 (2H)-yl)ethyl) piperidine-1-carboxylate: To a solution of tert-butyl 4-(2-(2-oxo-3-(3-(4, 4, 5, 5-tetramethyl-1, 3, 2-dioxaborolan-2-yl)phenyl) tetrahydropyrimidin-1 (2H)-yl)ethyl) piperidine-1-carboxylate (1200 mg, 2.33 mmol) in dioxane/H₂O (10:1, 30 mL) was added 5-bromo-4-chloro-3-ethyl-1H-pyrrolo[2, 3-b] pyridine (605 mg, 2.33 mmol), Na₂CO 3 (742 mg, 7.0 mmol) and Pd(dppf)Cl₂ (170 mg, 0.23 mmol). The reaction mixture was stirred at 90° C. under N₂ for 4 hrs. After the reaction completed, H₂O (20 mL) was added to the reaction mixture, and then extracted with DCM (20 mL×3). The combined organic layer was washed with brine (30 mL×2), then dried over anhydrous Na₂SO₄. After filtration, the solution was concentration under vacuum, and the residue was purified by Combiflash (DCM/MeOH-0˜ 10%) to give the product (600 mg, 45%) as yellow oil. Mass (m/z): 566.3 [M+H]⁺.

Step 2. Preparation of 1-(3-(4-chloro-3-ethyl-1H-pyrrolo[2, 3-b] pyridin-5-yl)phenyl)-3-(2-(piperidin-4-yl)ethyl)tetrahydropyrimidin-2 (1H)-one: To a solution of tert-butyl 4-(2-(3-(3-(4-chloro-3-ethyl-1H-pyrrolo[2, 3-b] pyridin-5-yl)phenyl)-2-oxotetrahydropyrimidin-1 (2H)-yl)ethyl) piperidine-1-carboxylate (600 mg, 1.06 mmol) in TFA/DCM (3:1, 10 mL). The reaction mixture was stirred at rt for 1 hour. The resulting mixture was concentrated to give the product (560 mg, purity: 89%) as brown oil. Mass (m/z): 466.2 [M+H]⁺.

Step 3. Preparation of 1-(3-{4-chloro-3-ethyl-1H-pyrrolo[2, 3-b] pyridin-3-yl} phenyl)-3-{2-[1-(2-fluoro-4-nitrophenyl) piperidin-4-yl] ethyl}-1, 3-diazinan-2-one: To a solution of 1-(3-{4-chloro-3-ethyl-1H-pyrrolo[2, 3-b] pyridin-3-yl} phenyl)-3-[2-(piperidin-4-yl)ethyl]-1, 3-diazinan-2-one (500 mg, 1.72 mmol) in DMSO (10 mL) was added 1, 2-difluoro-4-nitrobenzene (474 mg, 1.72 mmol) and DIEA (692 mg, 5.36 mmol). The reaction mixture was stirred at 80° C. under N₂ for 1 hour. After the reaction completed, H₂O (100 mL) was added to the reaction mixture, and then extracted with EA (50 mL×3). The combined organic layer was washed with brine (50 mL×3), then dried over anhydrous Na₂SO₄. After filtration, the solution was concentrated under vacuum, and the residue was purified by Combiflash (DCM/MeOH=0˜10%) to give the product 1-(3-{4-chloro-3-ethyl-1H-pyrrolo [2, 3-b] pyridin-3-yl} phenyl)-3-{2-[1-(2-fluoro-4-nitrophenyl) piperidin-4-yl] ethyl}-1, 3-diazinan-2-one as yellow solid (436 mg, 67%). Mass (m/z): 605.1 [M+H]⁺.

Step 4. Preparation of 1-{2-[1-(4-amino-2-fluorophenyl) piperidin-4-yl] ethyl}-3-(3-{4-chloro-3-ethyl-1H-pyrrolo [2, 3-b] pyridin-3-yl} phenyl)-1, 3-diazinan-2-one: A solution of 1-(3-{4-chloro-3-ethyl-1H-pyrrolo [2, 3-b] pyridin-3-yl} phenyl)-3-{2-[1-(2-fluoro-4-nitrophenyl) piperidin-4-yl] ethyl}-1, 3-diazinan-2-one (436 mg, 0.72 mmol) in EA (10 mL) was stirred at 80° C. for 16 hrs. The solvent was removed under reduced pressure and the residue was purified by Combiflash [DCM/MeOH (10% NH₃·H₂O)=0˜10%] to give the product 1-{2-[1-(4-amino-2-fluorophenyl) piperidin-4-yl] ethyl}-3-(3-{4-chloro-3-ethyl-1H-pyrrolo [2, 3-b] pyridin-3-yl} phenyl)-1, 3-diazinan-2-one as a brown solid (300 mg, 72%). Mass (m/z): 575.2 [M+H]⁺.

Step 5. Preparation of 3-{[4-(4-{2-[3-(3-{4-chloro-3-ethyl-1H-pyrrolo [2, 3-b]pyridin-3-yl} phenyl)-2-oxo-1, 3-diazinan-1-yl] ethyl} piperidin-1-yl)-3-fluorophenyl]amino} piperidine-2, 6-dione: To a solution of 1-{2-[1-(4-amino-2-fluorophenyl) piperidin-4-yl] ethyl}-3-(3-{4-chloro-3-ethyl-1H-pyrrolo [2, 3-b] pyridin-3-yl}phenyl)-1, 3-diazinan-2-one (250 mg, 0.43 mmol) in DMF (10 mL) was added 3-bromopiperidine-2, 6-dione (415 mg, 2.15 mmol) and NaHCO₃ (110 mg, 1.30 mmol). The reaction mixture was stirred at 80° C. under N₂ for 16 hrs. The solvent was removed under reduced pressure and the residue was purified by Combiflash (DCM/MeOH=0˜10%) to give the product 3-{[4-(4-{2-[3-(3-{4-chloro-3-ethyl-1H-pyrrolo [2, 3]-b] pyridin-3-yl}-phenyl)-2-oxo-1, 3-diazinan-1-yl]-ethyl} piperidin-1-yl)-3-fluorophenyl] amino} piperidine-2, 6-dione as a white solid (30 mg, 10%). Mass (m/z): 686.0 [M+H]⁺. ¹H NMR (400 MHz, MeOD) δ 8.10 (d, J=8.2 Hz, 1H), 7.46 (t, J=7.8 Hz, 1H), 7.38 (d, J=1.6 Hz, 1H), 7.32 (t, J=7.4 Hz, 2H), 7.25 (s, 1H), 6.88 (s, 1H), 6.56-6.42 (m, 2H), 4.61 (s, 1H), 4.23 (d, J=8.2 Hz, 1H), 3.81-3.74 (m, 2H), 3.47 (t, J=7.6 Hz, 4H), 3.22 (d, J=10.8 Hz, 2H), 3.00 (q, J=7.4 Hz, 2H), 2.85-2.72 (m, 2H), 2.62 (s, 2H), 2.29 (s, 1H), 2.20-2.12 (m, 2H), 1.86 (s, 2H), 1.59 (s, 2H), 1.44 (s, 2H), 1.32 (s, 4H).

### Example 17

### 3-[4-(4-{2-[3-(3-{4-chloro-3-ethyl-1H-pyrrolo [2, 3-b] pyridin-3-yl} phenyl)-2-oxo-1, 3-diazinan-1-yl] ethyl} piperazin-1-yl) phenyl] piperidine-2, 6-dione

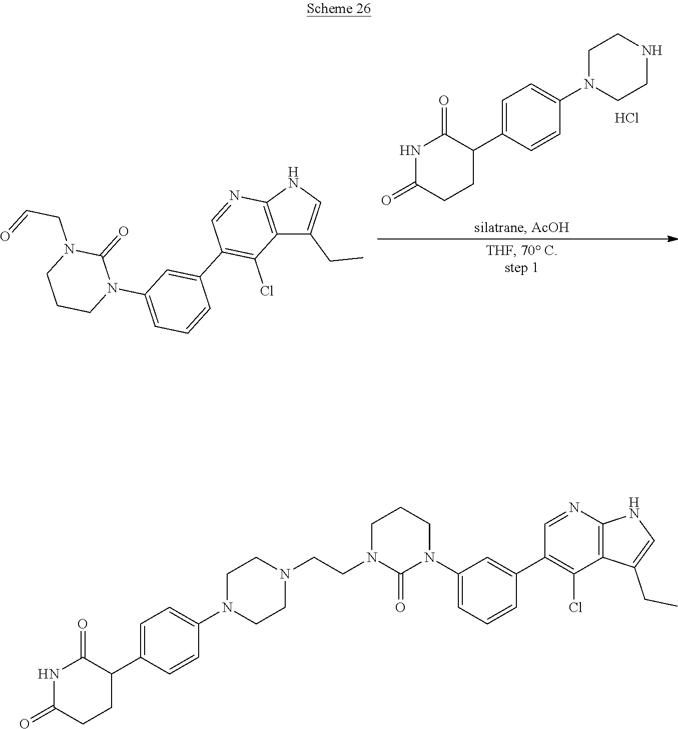 [SMILES: CCc1c[nH]c2ncc(-c3cccc(N4CCCN(CC=O)C4=O)c3)c(Cl)c12 ; O=C1CCC(c2ccc(N3CCNCC3)cc2)C(=O)N1 ; Cl ; CCc1c[nH]c2ncc(-c3cccc(N4CCCN(CCN5CCN(c6ccc(C7CCC(=O)NC7=O)cc6)CC5)C4=O)c3)c(Cl)c12]

To a solution of 2-[3-(3-{4-chloro-3-ethyl-1H-pyrrolo [2, 3-b] pyridin-3-yl}phenyl)-2-oxo-1, 3-diazinan-1-yl] acetaldehyde (200 mg, 0.50 mmol) in THF (5 mL) was added 3-(4-(piperazin-1-yl) phenyl) piperidine-2, 6-dione hydrochloride (156 mg, 0.50 mmol) and silatrane (265 mg, 1.5 mmol) and AcOH (one drop). The reaction mixture was stirred at 70° C. under N₂ for 18 hrs. The solution was concentrated under reduced pressure. The residue was purified by prep-HPLC [Gemini-C18, 150×21.2 mm, Sum; ACN-H₂O (0.1% FA), 20-50] to give the desired product (20 mg, 6%) as a white solid. Mass (m/z): 654.1 [M+H]⁺. ¹HNMR (400 MHz, MeOD) δ8.00 (s, 1H), 7.36 (t, J=7.8 Hz, 1H), 7.30 (d, J=1.6 Hz, 1H), 7.22 (t, J=7.8 Hz, 2H), 7.16 (s, 1H), 7.00 (d, J=8.6 Hz, 2H), 6.83 (d, J=8.6 Hz, 2H), 3.71-3.65 (m, 3H), 3.51 (t, J=6.8 Hz, 2H), 3.44 (t, J=5.8 Hz, 2H), 3.14-3.07 (m, 4H), 2.89 (q, J=7.4 Hz, 2H), 2.72 (s, 4H), 2.65 (t, J=6.6 Hz, 2H), 2.58-2.46 (m, 2H), 2.11-2.04 (m, 4H), 1.22 (t, J=7.4 Hz, 3H).

### Example 18

### 3-{[4-(4-{2-[3-(3-{4-chloro-3-ethyl-1H-pyrrolo [2, 3-b] pyridin-3-yl} phenyl)-2-oxo-1, 3-diazinan-1-yl] ethyl} piperazin-1-yl)-3-fluorophenyl] amino} piperidine-2, 6-dione

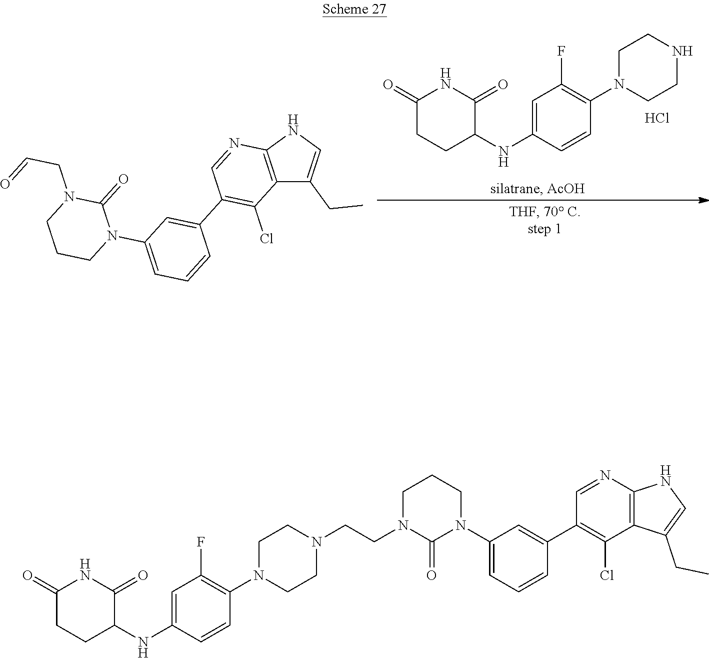 [SMILES: CCc1c[nH]c2ncc(-c3cccc(N4CCCN(CC=O)C4=O)c3)c(Cl)c12 ; O=C1CCC(Nc2ccc(N3CCNCC3)c(F)c2)C(=O)N1 ; Cl ; CCc1c[nH]c2ncc(-c3cccc(N4CCCN(CCN5CCN(c6ccc(NC7CCC(=O)NC7=O)cc6F)CC5)C4=O)c3)c(Cl)c12]

To a solution of 2-[3-(3-{4-chloro-3-ethyl-1H-pyrrolo [2, 3-b] pyridin-3-yl} phenyl)-2-oxo-1, 3-diazinan-1-yl] acetaldehyde (200 mg, 0.50 mmol) in THF (5 mL) was added 3-{[3-fluoro-4-(piperazin-1-yl) phenyl] amino} piperidine-2, 6-dione hydrochloride (173 mg, 0.50 mmol) and silatrane (265 mg, 1.5 mmol) and AcOH (one drop). The reaction mixture was stirred at 70° C. under N₂ for 18 hrs. The solution was concentrated under reduced pressure. The residue was purified by prep-HPLC [Gemini-C18, 150×21.2 mm, 5 um; ACN-H₂O (0.1% FA), 20-50] to give the desired product (8 mg, 2%) as a white solid. Mass (m/z): 687.2 [M+H]⁺. ¹H NMR (400 MHz, DMSO-d₆) δ 11.78 (s, 1H), 10.79 (s, 1H), 8.11 (s, 1H), 7.37 (dd, J=14.6, 8.4 Hz, 4H), 7.20 (d, J=7.4 Hz, 1H), 6.79 (s, 1H), 6.50 (d, J=14.8 Hz, 1H), 6.39 (d, J=8.6 Hz, 1H), 5.80 (d, J=7.6 Hz, 1H), 4.29-4.21 (m, 1H), 3.73-3.69 (m, 2H), 3.42 (d, J=6.0 Hz, 4H), 2.92 (q, J=7.4 Hz, 2H), 2.84 (s, 4H), 2.69 (d, J=11.2 Hz, 1H), 2.54 (s, 6H), 2.13-1.96 (m, 4H), 1.84 (dd, J=12.2, 4.4 Hz, 1H), 1.26 (t, J=7.4 Hz, 3H).

### General Assay Procedures:

### HPK1 Enzyme Inhibition Assay

The compound was dissolved in 100% DMSO at the concentration of 10 mM. The HPK1 protein was purchased from Signal Chem (M23-11G-10). 2.5 μL per well of 2×HPK1 protein was added to assay plate containing the test compound, centrifuged at 1500 rpm for 1 minute, and then incubated at 25° C. for 60 minutes. MBP protein was purchased from Signal Chem (M42-51N) and ATP was purchased from Promega (V9102). The two were added 2.5 μL per well mixture of 2×MBP (0.2 ug/ul) and ATP (20 μM), centrifuged at 1500 rpm for 1 minute, then incubated at 25° C. for 60 minutes. Then added 5 μL of ADP-Glo from Promega (V9102) to the assay plate and depleted the unconsumed ATP for 60 minutes. Then centrifuged at 1500 rpm for 1 minute and incubated at 25° C. for 60 minutes. Finally, 10 μL of the kinase assay reagent from Promega (V9102) was added to the assay plate to convert ADP to ATP, centrifuged at 1500 rpm for 1 minute, incubate at 25° C. for 40 minutes. After 40 minutes incubation, the fluorescence was determined. Based on the results, the IC₅₀ value of the compound was calculated. The results of IC₅₀ are shown in the following Table 2.

### FLT3-ITD Enzyme Inhibition Assay

The compound was dissolved in 100% DMSO at the concentration of 10 mM. The FLT3-ITD protein was purchased from Invitrogen (PV6191). 10 μL per well of 2.5×FLT3-ITD protein was added to assay plate containing the test compound, centrifuged at 1000 rpm for 1 minute, and then incubated at 25° C. for 10 minutes. Peptide 2 was purchased from GL Biochem (112394) and ATP was purchased from Promega (V9102). The two were added 15 μL per well mixture of 1.67× peptide 2 (final conc. is 3 μM) and ATP (final conc. is 97.2 μM), centrifuged at 1000 rpm for 1 minute, then incubated at 25° C. for 40 minutes. Then added 30 μL of stop buffer (100 mM HEPES pH7.5, 0.015% Brij-35, 0.2% Coating Reagent 3, 50 mM EDTA) to the assay plate and centrifuged at 1000 rpm for 1 minute. the product was determined. Based on the results, the IC₅₀ value of the compound was calculated. The results of IC₅₀ are shown in the following Table 2.

### Aurora A Enzyme Inhibition Assay

The compound was dissolved in 100% DMSO at the concentration of 10 mM. The Aurora A protein was purchased from Carna (05-101). 10 μL per well of 2.5×Aurora A protein was added to assay plate containing the test compound, centrifuged at 1000 rpm for 1 minute, and then incubated at 25° C. for 10 minutes. Peptide 21 was purchased from GL Biochem (116370) and ATP was purchased from Promega (V9102). The two were added 15 μL per well mixture of 1.67× peptide 21 (final conc. is 3 μM) and ATP (final conc. is 14.58 μM), centrifuged at 1000 rpm for 1 minute, then incubated at 25° C. for 40 minutes. Then added 30 μL of stop buffer (100 mM HEPES pH7.5, 0.015% Brij-35, 0.2% Coating Reagent 3, 50 mM EDTA) to the assay plate and centrifuged at 1000 rpm for 1 minute. the product was determined. Based on the results, the IC₅₀ value of the compound was calculated. The results of IC₅₀ are shown in the following Table 2.

TABLE 2
Results of compounds in enzyme inhibition assays

| | HPK1 IC₅₀ | FLT3-ITD IC₅₀ | Aurora A IC₅₀ |
|---|---|---|---|
| Example | (nM) | (nM) | (nM) |
| 1 | 0.76 | 4.0 | 14.3 |
| 2 | 1.1 | 4.7 | 10.1 |
| 3 | 5.8 | 13.6 | 50.8 |
| 4 | 17.1 | 24.0 | 25.2 |
| 5 | 13.4 | 27.7 | 21.8 |
| 6 | 3.1 | 18.4 | 9.9 |
| 7 | 10.5 | 12.3 | 10.6 |
| 8 | 15.7 | 5.7 | 110 |
| 9 | 62.9 | 36.5 | 130.8 |
| 10 | 30.7 | 7.4 | 34.8 |
| 11 | 13.6 | 20.8 | 21.2 |
| 12 | 34.3 | N.D. | N.D. |
| 13 | 12.5 | 23.8 | 14.3 |
| 14 | 25.8 | N.D. | N.D. |
| 15 | 4.6 | 8.8 | 19.9 |
| 16 | 5.5 | 12.3 | 40.8 |
| 17 | 2.9 | 17.4 | 16.6 |
| 18 | 2.5 | 5.4 | 10.6 |

N.D.: Not Determined
HPK1 degradation assay

Frozen human PBMC were purchased from Shanghai OribioTech and recovered with culture medium (RMPI1640) prior to use. The cells were then incubated with a variety of concentrations of compound. After incubation, the cells were collected and lysed. The protein concentration was determined by BCA protein assay kit from Thermo (23227). The HPK1 protein level was determined by western blots, using anti-human HPK1 polyclonal antibody from CST (4472S). Proteins were loaded into each well of the pre-casting gels and subjected to electrophoretic separation by SDS-PAGE. The protein resolved by SDS-PAGE were transferred to PVDF, blocked by 5% skim milk, and probed with anti-human HPK1 antibody or β-actin antibody from CST (3700S), using standard western blotting procedure.

### MEK 1/2 Degradation Assay

MV-411 cells were cultured with medium (IMDM) and IMR32 cells were cultured with medium (MEM) prior to use. Cells were then incubated with a variety of concentrations of compound. After incubation, the cells were collected and lysed. The protein concentration was determined by BCA protein assay kit from Thermo (23227). The FLT3 protein level was determined by western blots, using anti-human MEK 1/2 polyclonal antibody from CST (9122S). Proteins were loaded into each well of the pre-casting gels and subjected to electrophoretic separation by SDS-PAGE. The protein resolved by SDS-PAGE were transferred to PVDF, blocked by 5% skim milk, and probed with anti-human MEK1/2 antibody or β-actin antibody from CST (3700S) and COX IV antibody from CST (4850S), using standard western blotting procedure.

### FLT3 Degradation Assay

MV-411 cells were cultured with medium (IMDM) prior to use. Cells were then incubated with a variety of concentrations of compound. After incubation, the cells were collected and lysed. The protein concentration was determined by BCA protein assay kit from Thermo (23227). The FLT3 protein level was determined by western blots, using anti-human FLT3 monoclonal antibody from CST (3462S). Proteins were loaded into each well of the pre-casting gels and subjected to electrophoretic separation by SDS-PAGE. The protein resolved by SDS-PAGE were transferred to PVDF, blocked by 5% skim milk, and probed with anti-human FLT3 antibody or -actin antibody from CST (3700S), using standard western blotting procedure.

### Aurora A Degradation Assay

IMR32 cells were cultured with medium (MEM), Huh7 cells were cultured with medium (DMEM) and HL-60 cells were culture with medium (IMDM) prior to use. Cells were then incubated with a variety of concentrations of compound. After incubation, the cells were collected and lysed. The protein concentration was determined by BCA protein assay kit from Thermo (23227). The AURKA protein level was determined by western blots, using anti-human AURKA monoclonal antibody from CST (14475S). Proteins were loaded into each well of the pre-casting gels and subjected to electrophoretic separation by SDS-PAGE. The protein resolved by SDS-PAGE were transferred to PVDF, blocked by 5% skim milk, and probed with anti-human AURKA antibody or β-actin antibody from CST (3700S) or COX IV antibody from CST (4850S), using standard western blotting procedure.

The results of the HPK1, FLT3, Aurora, MEK are shown in Table 3.

TABLE 3
Results of compounds in protein degradation assays

| | HPK1 DC₅₀ | FLT3 DC₅₀ | Aurora A DC₅₀ | MEK 1/2 DC₅₀ |
|---|---|---|---|---|
| | (nM)/ | (nM)/ | (nM)/ | (nM)/ |
| Example | Dmax (%) | Dmax (%) | Dmax (%) | Dmax (%) |
| 1 | 173/75 | + | + | 68/20 |
| 2 | 3/80 | + | + | + |
| 4 | + | + | + | <1/30 |
| 5 | + | + | + | 2.1/65 |
| 6 | + | + | + | <1/32 |
| 7 | 12/60 | + | 5/50 | 264/37 |
| 8 | 85/95 | 8/95 | <20/95 | 82/91 |
| 9 | 294/50 | 100/60 | >1000/50 | + |
| 11 | + | + | 9/50 | 49/30 |
| 13 | 11/50 | + | 3/70 | <1/30 |
| 14 | 100/90 | 11/95 | <20/95 | <100/90 |
| 15 | 6/80 | 0.02/34 | 9.6/82 | 1/38 |
| 16 | 100/64 | 115/17 | 4.6/75 | 4.6/75 |
| 17 | 2.3/91 | 8.7/80 | 1.0/50 | 1/47 |
| 18 | 1.0/52 | + | 1.1/74 | 1/74 |

+: No activities detected up to 10 uM.

### OTHER EMBODIMENTS

The present disclosure provides merely exemplary embodiments. One skilled in the art will readily recognize from the present disclosure and claims, that various changes, modifications and variations can be made therein without departing from the spirit and scope of the present disclosure as defined in the following claims.

1. A compound of Formula (I): 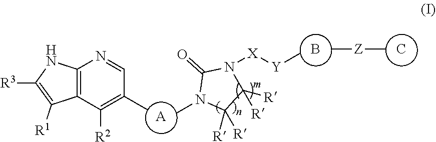
a tautomer thereof, a deuterated derivative of the compound or the tautomer, or a pharmaceutically acceptable salt of the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R;
(ii) R² and R³ are independently chosen from hydrogen, halogen groups, OR^{x}, SR^{x}, NHR^{x}, N(R^{x})₂, CHR^{x}, and C(R^{x})₂;
(iii) each R′ is independently chosen from hydrogen, halogen groups, linear, branched, and cyclic alkyl groups;
(iv) m and n are independently chosen from 0, 1, and 2;
(v) X is absent or is chosen from linear, branched, cyclic alkylene groups, linear, branched, and cyclic heteroalkylene groups;
(vi) Y and Z are independently absent or chosen from —O—, —C(O)—, —C(O)R^{x}—, —C(S)—, —C(S)R^{x}—, —[C(R^{x}R^{y})]ₚ—, —S(O)₂—, —S(O)₂R^{x}—, NR^{x}—, and —NR^{x}C(O)—, wherein p is chosen from 1, 2, 3, 4, 5, and 6;
wherein if X is absent, then Y is not —O—, —S(O)₂—, —S(O)₂R^{x}—, NR^{x}—, or —NR^{x}C(O)—;
(vii) R^{x}, R^{y}, and R^{w} are each independently chosen from hydrogen, linear, branched, and cyclic alkyl groups, carbocyclic groups, heterocyclic groups, aryl groups, and heteroaryl groups;
(viii) ring A is chosen from optionally substituted aryl groups and heteroaryls groups,
(ix) ring B is absent or is chosen from cycloalkyl groups and heterocycloalkyls;
(x) ring C is chosen
from 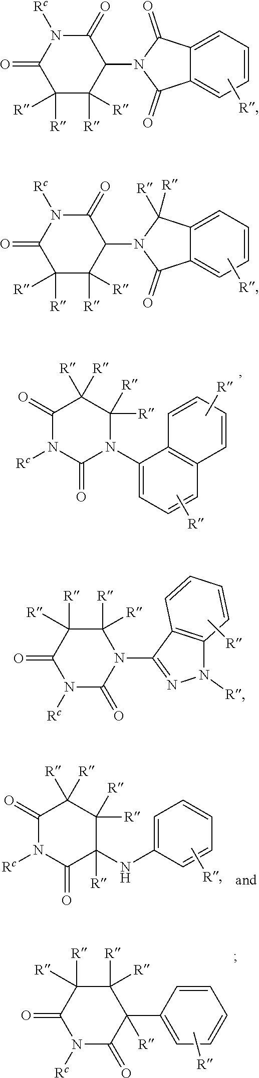
wherein R^{c} is chosen from hydrogen, linear, branched, and cyclic alkyl groups, and pro-drug groups; R″ is chosen from hydrogen, halogen groups, OR^{x}, linear, branched, and cyclic alkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O) C₁-C₆ linear, branched, and cyclic alkyl groups,
—C(O) OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O) C₁-C₆ linear, branched, and cyclic alkyl groups,
—C(O) NHC₁-C₆ linear, branched, and cyclic alkyl groups,
—NHaryl groups,
—N(aryl groups)₂,
—NHC(O) aryl groups,
—C(O) NHaryl groups,
—NHheteroaryl groups,
—N(heteroaryl groups)₂,
—NHC(O) heteroaryl groups,
—C(O) NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

2. The compound, tautomer, deuterated derivative, or pharmaceutically acceptable salt of claim 1, wherein R¹ is chosen from linear, branched, and cyclic alkyl groups; R² is a halogen group; and R³ is chosen from hydrogen, linear, branched, and cyclic alkyl groups.

3. The compound, tautomer, deuterated derivative, or pharmaceutically acceptable salt of claim 1 or 2, wherein R¹ is chosen from C₁-C₆ linear, branched, and cyclic alkyl groups.

4. The compound, tautomer, deuterated derivative, or pharmaceutically acceptable salt of claim 3, wherein R¹ is chosen from methyl, ethyl, cyclopropyl, and cyclobutyl.

5. The compound, tautomer, deuterated derivative, or pharmaceutically acceptable salt of any of claims 1-4, wherein R² is a halogen group.

6. The compound, tautomer, deuterated derivative, or pharmaceutically acceptable salt of claim 5, wherein R² is chloro.

7. The compound, tautomer, deuterated derivative, or pharmaceutically acceptable salt of any of claims 1-5, wherein R² is hydrogen.

8. The compound, tautomer, deuterated derivative, or pharmaceutically acceptable salt of any of claims 1-7, wherein R³ is a halogen group.

9. The compound, tautomer, deuterated derivative, or pharmaceutically acceptable salt of claim 8, wherein R³ is chloro.

10. The compound, tautomer, deuterated derivative, or pharmaceutically acceptable salt of any of claims 1-7, wherein R³ is hydrogen.

11. The compound, tautomer, deuterated derivative, or pharmaceutically acceptable salt of any of claims 1-10, wherein m is 1 and n is 1.

12. The compound, tautomer, deuterated derivative, or pharmaceutically acceptable salt of claim 11, wherein each R′ is hydrogen.

13. The compound, tautomer, deuterated derivative, or pharmaceutically acceptable salt of any of claims 1-10, wherein m is 2 and n is 1.

14. The compound, tautomer, deuterated derivative, or pharmaceutically acceptable salt of claim 11, wherein each R′ is hydrogen.

15. The compound, tautomer, deuterated derivative, or pharmaceutically acceptable salt of any of claims 1-14, wherein X is absent.

16. The compound, tautomer, deuterated derivative, or pharmaceutically acceptable salt of any of claims 1-14, wherein X is a linear alkylene group.

17. The compound, tautomer, deuterated derivative, or pharmaceutically acceptable salt of claim 16, wherein X is a methylene group.

18. The compound, tautomer, deuterated derivative, or pharmaceutically acceptable salt of claim 16, wherein X is an ethylene group.

19. The compound, tautomer, deuterated derivative, or pharmaceutically acceptable salt of any of claims 1-18, wherein Y is absent.

20. The compound, tautomer, deuterated derivative, or pharmaceutically acceptable salt of any of claims 1-19, wherein ring B is chosen from optionally substituted heterocycloalkyls.

21. The compound, tautomer, deuterated derivative, or pharmaceutically acceptable salt of claim 20, wherein ring B is chosen from

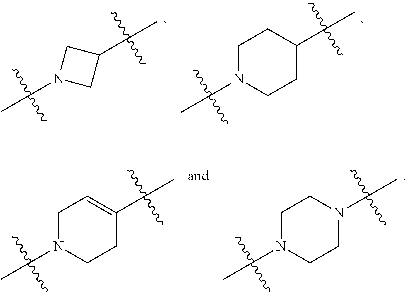 [SMILES: *C1CN(*)C1 ; *C1CCN(*)CC1 ; *C1=CCN(*)CC1 ; *N1CCN(*)CC1]

22. The compound, tautomer, deuterated derivative, or pharmaceutically acceptable salt of any of claims 1-21, wherein Z is absent.

23. The compound, tautomer, deuterated derivative, or pharmaceutically acceptable salt of any of claims 1-22, wherein ring C is

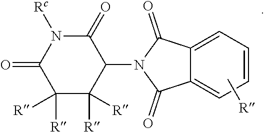

24. The compound, tautomer, deuterated derivative, or pharmaceutically acceptable salt of claim 23, wherein R^{c} is chosen from hydrogen, linear, branched, and cyclic alkyl groups, and pro-drug groups.

25. The compound, tautomer, deuterated derivative, or pharmaceutically acceptable salt of any of claims 1-22, wherein ring C is

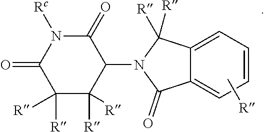

26. The compound, tautomer, deuterated derivative, or pharmaceutically acceptable salt of claim 25, wherein R^{c} is chosen from hydrogen, linear, branched, and cyclic alkyl groups, and pro-drug groups.

27. The compound, tautomer, deuterated derivative, or pharmaceutically acceptable salt of any of claims 1-22, wherein ring C is

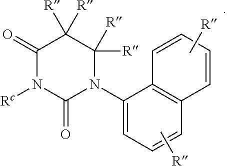

28. The compound, tautomer, deuterated derivative, or pharmaceutically acceptable salt of claim 27, wherein R^{c} is chosen from hydrogen, linear, branched, and cyclic alkyl groups, and pro-drug groups.

29. The compound, tautomer, deuterated derivative, or pharmaceutically acceptable salt of any of claims 1-22, wherein ring C is

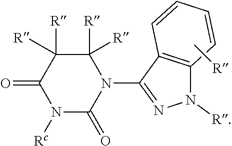

30. The compound, tautomer, deuterated derivative, or pharmaceutically acceptable salt of claim 29, wherein R^{c} is chosen from hydrogen, linear, branched, and cyclic alkyl groups,
and pro-drug groups.

31. The compound, tautomer, deuterated derivative, or pharmaceutically acceptable salt of any of claims 1-22, wherein ring C is

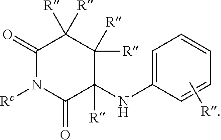

32. The compound, tautomer, deuterated derivative, or pharmaceutically acceptable salt of claim 31, wherein R^{c} is chosen from hydrogen, linear, branched, and cyclic alkyl groups, and pro-drug groups.

33. The compound, tautomer, deuterated derivative, or pharmaceutically acceptable salt of any of claims 1-22, wherein ring C is

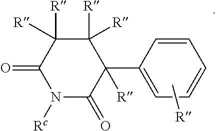

34. The compound, tautomer, deuterated derivative, or pharmaceutically acceptable salt of claim 33, wherein R^{c} is chosen from hydrogen, linear, branched, and cyclic alkyl groups, and pro-drug groups.

35. A compound chosen from 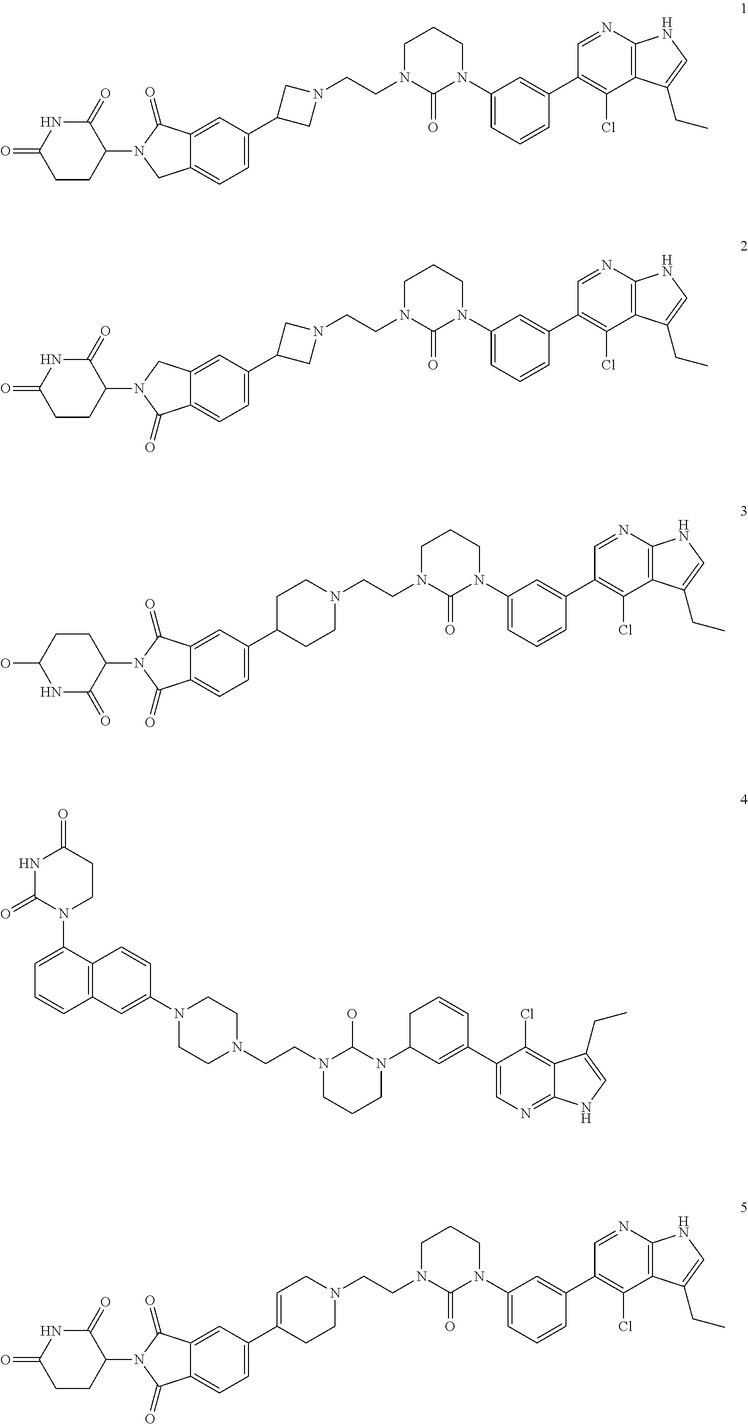 [SMILES: CCc1c[nH]c2ncc(-c3cccc(N4CCCN(CCN5CC(c6ccc7c(c6)C(=O)N(C6CCC(=O)NC6=O)C7)C5)C4=O)c3)c(Cl)c12 ; CCc1c[nH]c2ncc(-c3cccc(N4CCCN(CCN5CC(c6ccc7c(c6)CN(C6CCC(=O)NC6=O)C7=O)C5)C4=O)c3)c(Cl)c12 ; CCc1c[nH]c2ncc(-c3cccc(N4CCCN(CCN5CCC(c6ccc7c(c6)C(=O)N(C6CCC(O)NC6=O)C7=O)CC5)C4=O)c3)c(Cl)c12 ; CCc1c[nH]c2ncc(C3=CC(N4CCCN(CCN5CCN(c6ccc7c(N8CCC(=O)NC8=O)cccc7c6)CC5)C4O)CC=C3)c(Cl)c12 ; CCc1c[nH]c2ncc(-c3cccc(N4CCCN(CCN5CC=C(c6ccc7c(c6)C(=O)N(C6CCC(=O)NC6=O)C7=O)CC5)C4=O)c3)c(Cl)c12] 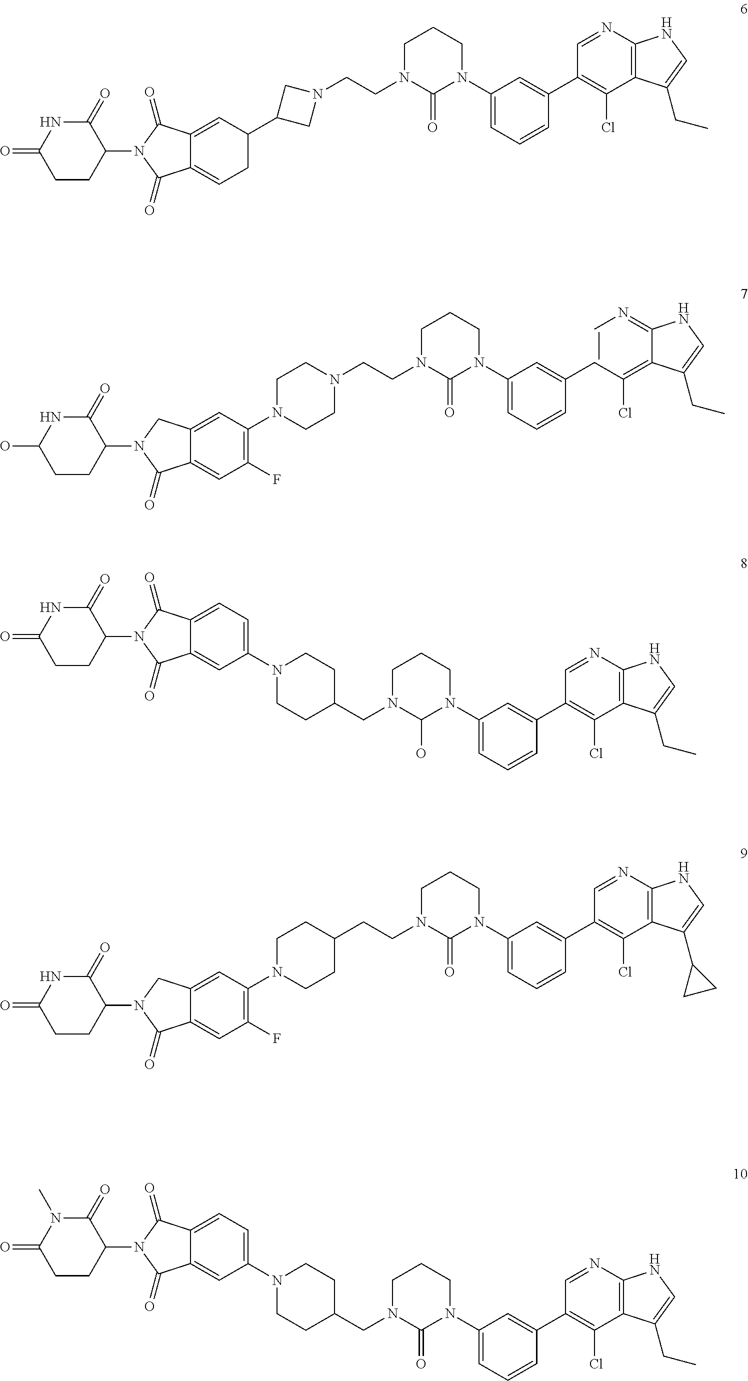 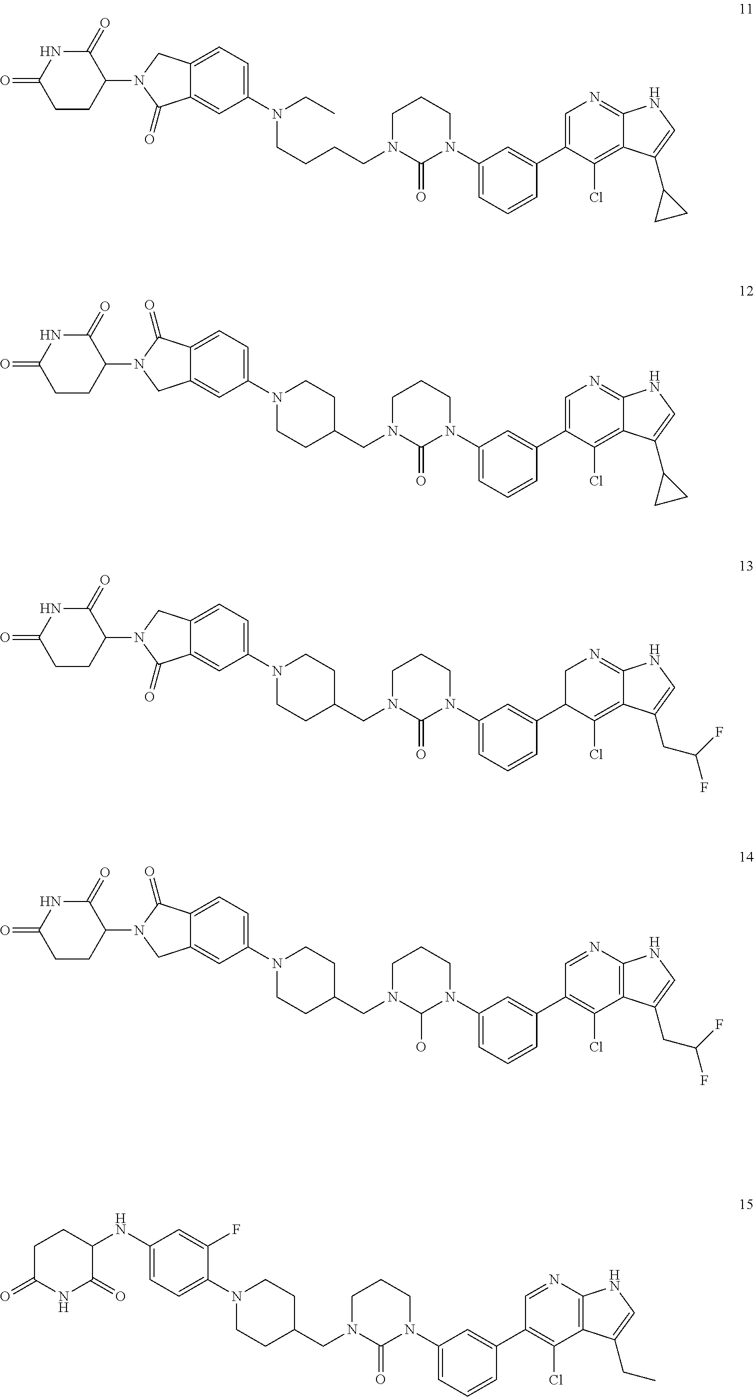 [SMILES: CCN(CCCCN1CCCN(c2cccc(-c3cnc4[nH]cc(C5CC5)c4c3Cl)c2)C1=O)c1ccc2c(c1)C(=O)N(C1CCC(=O)NC1=O)C2 ; O=C1CCC(N2Cc3cc(N4CCC(CN5CCCN(c6cccc(-c7cnc8[nH]cc(C9CC9)c8c7Cl)c6)C5=O)CC4)ccc3C2=O)C(=O)N1 ; O=C1CCC(N2Cc3ccc(N4CCC(CN5CCCN(c6cccc(C7CN=c8[nH]cc(CC(F)F)c8=C7Cl)c6)C5=O)CC4)cc3C2=O)C(=O)N1 ; O=C1CCC(N2Cc3cc(N4CCC(CN5CCCN(c6cccc(-c7cnc8[nH]cc(CC(F)F)c8c7Cl)c6)C5O)CC4)ccc3C2=O)C(=O)N1 ; CCc1c[nH]c2ncc(-c3cccc(N4CCCN(CC5CCN(c6ccc(NC7CCC(=O)NC7=O)cc6F)CC5)C4=O)c3)c(Cl)c12] 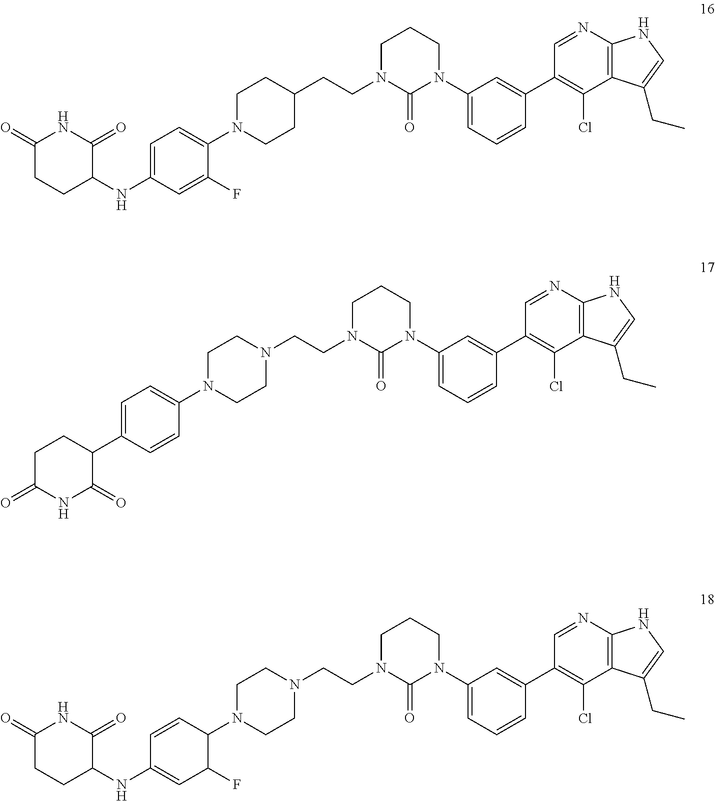 [SMILES: CCc1c[nH]c2ncc(-c3cccc(N4CCCN(CCC5CCN(c6ccc(NC7CCC(=O)NC7=O)cc6F)CC5)C4=O)c3)c(Cl)c12 ; CCc1c[nH]c2ncc(-c3cccc(N4CCCN(CCN5CCN(c6ccc(C7CCC(=O)NC7=O)cc6)CC5)C4=O)c3)c(Cl)c12 ; CCc1c[nH]c2ncc(-c3cccc(N4CCCN(CCN5CCN(C6C=CC(NC7CCC(=O)NC7=O)=CC6F)CC5)C4=O)c3)c(Cl)c12]
a tautomer thereof, a deuterated derivative of the compound or the tautomer, or a pharmaceutically acceptable salt of the foregoing.

36. A pharmaceutical composition comprising a compound, tautomer, deuterated derivative, and/or pharmaceutically acceptable salt according to any one of claims 1-35 and at least one pharmaceutically acceptable carrier.

37. A method for treating or alleviating a disease, a disorder or a condition mediated by the degradation of a protein kinase, comprising administering to a subject in need thereof a therapeutically effective amount of a compound, tautomer, deuterated derivative, and/or pharmaceutically acceptable salt according to any one of the claims 1-35 or the pharmaceutical composition according to claim 36.

38. The method of claim 37, wherein the protein kinase is chosen from hematopoietic progenitor kinase 1 (HPK1), mitogen-activated protein kinases 1/2 (MEK 1/2), Fms-like tyrosine kinase 3 receptor (FLT3), and Aurora A.

39. A method for decreasing a protein kinase activity in a disease, a disorder or a condition, comprising administering to a subject in need thereof a therapeutically effective amount of a compound, tautomer, deuterated derivative, and/or pharmaceutically acceptable salt according to any one of the claims 1-35 or the pharmaceutical composition according to claim 36.

40. The method of claim 39, wherein the disease, the disorder, or the condition is chosen from a protein kinase-related disease.

41. The method of claim 40, wherein the protein kinase-related disease is cancer.

42. The method of claim 41, wherein the cancer is a solid tumor.

43. The method of claim 42, wherein the solid tumor is chosen from brain cancer, breast cancer, respiratory tract and/or lung cancer, a reproductive organ cancer, bone cancer, digestive tract cancer, urinary tract cancer, eye cancer, liver cancer, skin cancer, head and neck cancer, anal cancer, nervous system cancer, thyroid cancer, and parathyroid cancer.

44. The method of claim 41, wherein the cancer is a hematologic cancer.

45. The method of claim 44, wherein the hematologic cancer is chosen from acute myeloid leukemia (AML), acute lymphoblastic leukemia (ALL), multiple myeloma (MM), diffuse large B-cell lymphoma (DLBCL), non-Hodgkin's lymphoma (NHL), Hodgkin's lymphoma mesothelioma (HL), T-cell lymphoma (TCL), Burkitt lymphoma (BL), chronic lymphocytic leukemia/small lymphocytic lymphoma (CLL/SLL), mantle cell lymphoma (MCL), marginal zone lymphoma (MZL), and myelodysplastic syndromes (MDS).

46. The method of claim 41, wherein the cancer is chosen from epidermoid oral such as buccal cavity, lip, tongue, mouth, pharynx; cardiac cancers such as sarcoma (angiosarcoma, fibrosarcoma, rhabdomyosarcoma, liposarcoma), myxoma, rhabdomyoma, fibroma, lipoma, and teratoma; lung cancers such as bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatosis hamartoma, mesothelioma; gastrointestinal cancers such as esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Kaposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers such as hepatoma (hepatocellular carcinoma), cholangiocarcinoma, hepatoblastoma, angiosarcoma, hepatocellular adenoma, hemangioma, biliary passages; bone cancers such as osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; cancers of the nervous system, including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosathecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers such as blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma) hairy cell; lymphoid disorders; skin cancers including malignant melanoma, basal cell carcinoma, squamous cell carcinoma, Kaposi's sarcoma, keratoacanthoma, moles dysplastic nevi, lipoma, angioma, dermatofibroma, keloids, psoriasis; cancers of the thyroid gland such as papillary thyroid carcinoma, follicular thyroid carcinoma; medullary' thyroid carcinoma, undifferentiated thyroid cancer, multiple endocrine neoplasia type 2A, multiple endocrine neoplasia type 2B, familial medullary thyroid cancer, pheochromocytoma, paraganglioma; and cancers of the adrenal glands like neuroblastoma.

47. The method of claim 39, further comprising the administration to the subject an existing standard treatment or an FDA-approved therapy.

48. The method of claim 39, further comprising the administration to the subject one or more separate pharmaceutical agents.

49. The method of claim 48, wherein the separate pharmaceutical agent is chosen from a chemotherapeutic agent, an immunotherapeutic agent, and an adjunctive therapeutic agent.
